(19) United States
(12) Patent Application Publication
(10) Pub. No.: US 2022/0029106 A1
(43) Pub. Date: Jan. 27, 2022

(54) ORGANIC ELECTROLUMINESCENT ELEMENT AND POLYCYCLIC COMPOUND FOR ORGANIC ELECTROLUMINESCENT ELEMENT

(71) Applicant: Samsung Display Co., Ltd., Yongin-si (KR)

(72) Inventors: Hyunah UM, Seoul (KR); Hyeongmin KIM, Suwon-si (KR); Heechoon AHN, Seoul (KR); Yeseul LEE, Busan (KR); Hyoyoung LEE, Suwon-si (KR); Yirang IM, Daejeon (KR); Seowon CHO, Asan-si (KR)

(21) Appl. No.: 17/219,567

(22) Filed: Mar. 31, 2021

(30) Foreign Application Priority Data
Jul. 14, 2020 (KR) 10-2020-0086868

(51) Int. Cl. H01L 51/00; C07D 209/86; C07D 471/04

(52) U.S. Cl. CPC H01L 51/0072; H01L 51/5056; C07D 471/04; C07D 209/86

(57) ABSTRACT

An organic electroluminescent element includes a first electrode, a second electrode, and an emission layer disposed between the first electrode and the second electrode, wherein the emission layer includes a polycyclic compound represented by Formula 1. The organic electroluminescent element may exhibit high efficiency and/or long service life:

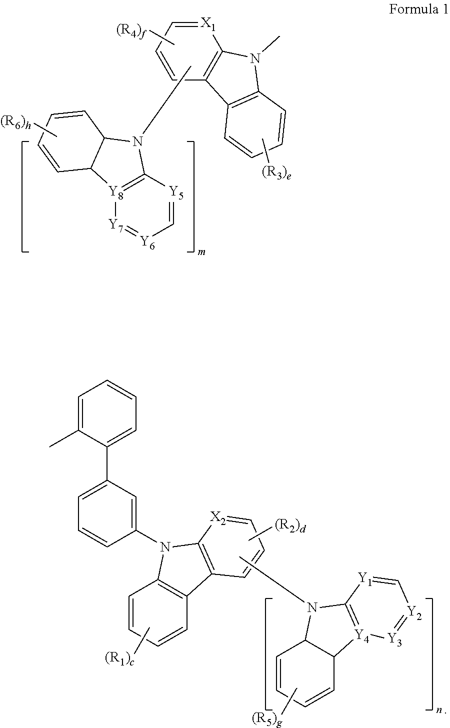

### CROSS-REFERENCE TO RELATED APPLICATION

This U.S. patent application claims priority to and the benefit of Korean Patent Application No. 10-2020-0086868, filed on Jul. 14, 2020, the entire content of which is hereby incorporated by reference.

### BACKGROUND

One or more aspects of embodiments of the present disclosure relate to an organic electroluminescent element and a polycyclic compound used therein, and for example, to a polycyclic compound used as a light-emitting material and an organic electroluminescent element including the same.

Organic electroluminescence displays are being actively developed as image display devices. Unlike liquid crystal displays and/or the like, an organic electroluminescence display device is a so-called self-luminescent display device, in which holes and electrons injected from a first electrode and a second electrode recombine in an emission layer so that an organic light-emitting material in the emission layer emits light to achieve display.

In the application of an organic electroluminescent element to a display, it is desired that the organic electroluminescent element has low driving voltage, high luminous efficiency, and/or long service life, and continuous development of a material for an organic electroluminescent element that can stably achieve the requirements is desired.

### SUMMARY

One or more aspects of embodiments of the present disclosure are directed toward an organic electroluminescent element that exhibits desired or excellent luminous efficiency, and a polycyclic compound used therein.

One or more example embodiments of the present disclosure provide an organic electroluminescent element including a first electrode, a second electrode facing the first electrode, and a plurality of functional layers disposed between the first electrode and the second electrode, wherein at least one of the functional layers includes a polycyclic compound, and the polycyclic compound includes a first benzene ring and a second benzene ring, which are linked by a single bond, a first carbazole group substituted in an ortho position of the first benzene ring with respect to the single bond, a second carbazole compound substituted in a meta position of the second benzene ring with respect to the single bond, and a third carbazole group substituted on at least one of the first carbazole group or the second carbazole group.

In an embodiment, the first carbazole group may be in an opposite position to the second group compound with respect to the single bond.

In an embodiment, the functional layers may include a hole transport region, an emission layer, and an electron transport region, and the emission layer may include the polycyclic compound.

In an embodiment, the emission layer may be to emit blue light.

In an embodiment, the emission layer may include a host and a dopant, and the host may include the polycyclic compound.

In an embodiment, the first carbazole group to the third carbazole group may each independently be an unsubstituted carbazole group, or a carbazole group that is substituted with at least one selected from a deuterium atom, a halogen atom, a substituted or unsubstituted silyl group, a cyano group, a substituted or unsubstituted alkyl group having 1 to 10 carbon atoms, a substituted or unsubstituted ring-forming aryl group having 6 to 30 carbon atoms, and a substituted or unsubstituted ring-forming heteroaryl group having 2 to 30 carbon atoms.

In an embodiment, the first carbazole group to the third carbazole group may each independently be represented by one selected from Formula C1 to Formula C10.

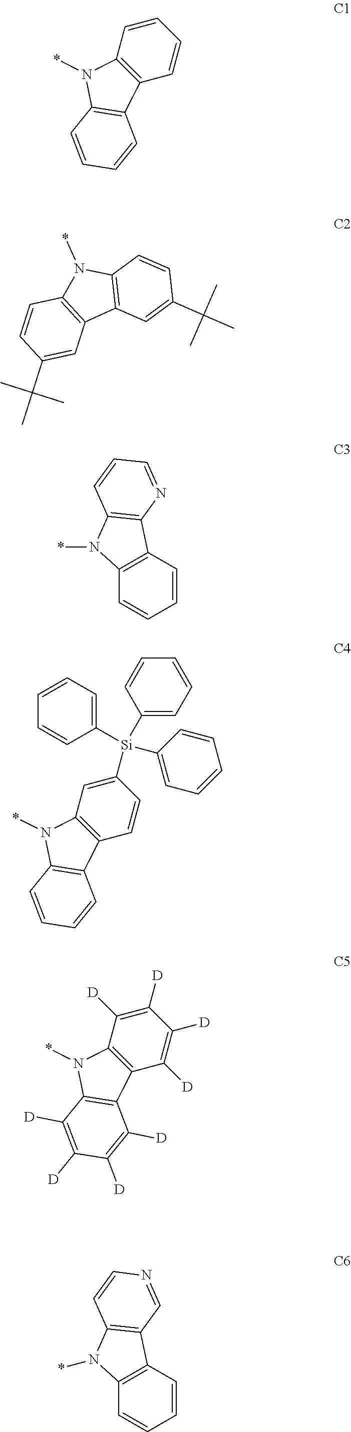

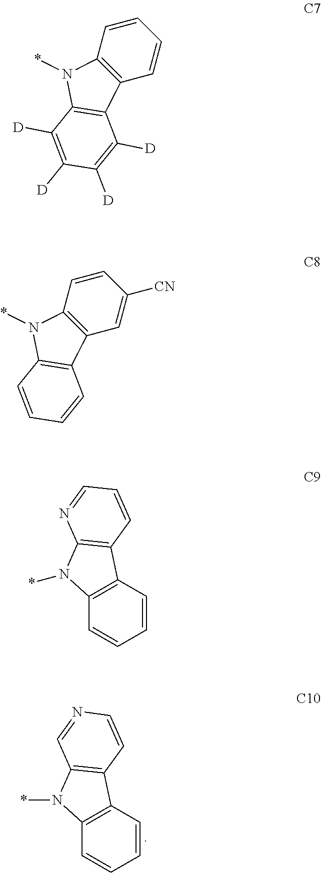 [SMILES: *n1c2ccccc2c2c([2H])c([2H])c([2H])c([2H])c21 ; *n1c2ccccc2c2cc(C#N)ccc21 ; *n1c2ccccc2c2cccnc21 ; *n1c2ccccc2c2ccncc21]

In an embodiment, the organic electroluminescent element includes a first electrode, a second electrode facing the first electrode, and a plurality of functional layers disposed between the first electrode and the second electrode, wherein at least one of the functional layers includes a polycyclic compound represented by Formula 1:

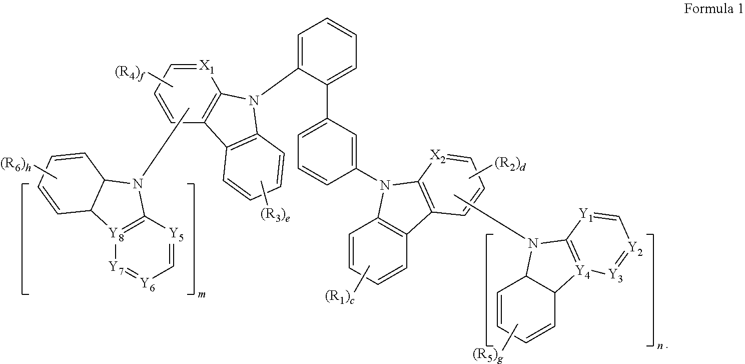

In Formula 1, “m” and “n” are each independently 0 or 1, “m+n” (e.g., the sum of m and n) is 1 or more, “c”, “e”, “g”, and “h” are each independently an integer of 0 to 4, “d” and “f” are each independently an integer of 0 to 3, X₁ and X₂ are each independently CRₐ or N, Y₁ to Y₈ are each independently CR_{b} or N, and Rₐ, R_{b}, and R₁ to R₆ are each independently a hydrogen atom, a deuterium atom, a halogen atom, a cyano group, a substituted or unsubstituted silyl group, a substituted or unsubstituted alkyl group having 1 to 10 carbon atoms, a substituted or unsubstituted ring-forming aryl group having 6 to 30 carbon atoms, or a substituted or unsubstituted ring-forming heteroaryl group having 2 to 30 carbon atoms.

In an embodiment, the polycyclic compound represented by Formula 1 may be represented by Formula 2-1 or Formula 2-2:

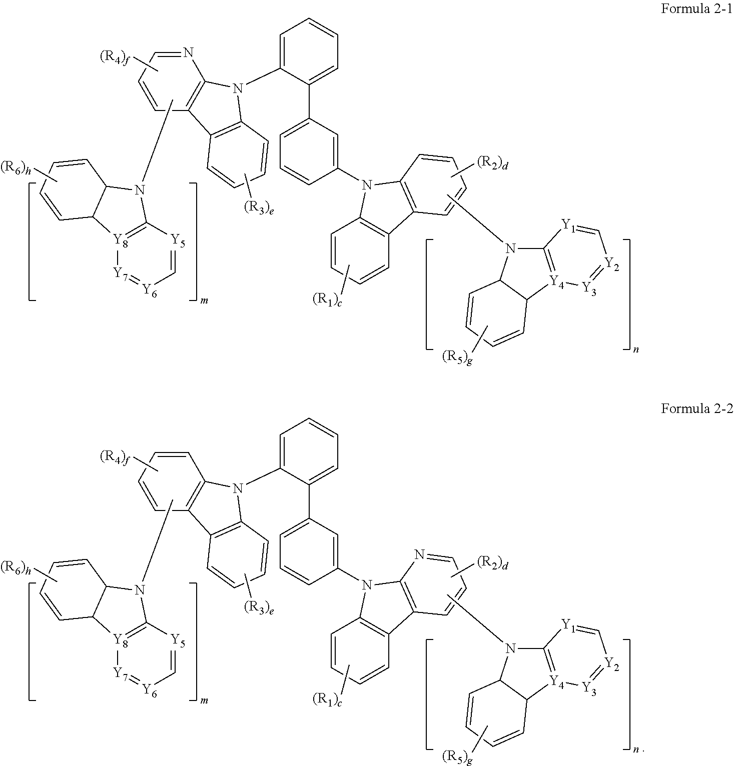

In Formula 2-1 and Formula 2-2, “m”, “n”, “c” to “h”, R₁ to R₆, and Y₁ to Y₈ may each independently be the same as defined in Formula 1.

In an embodiment, the polycyclic compound represented by Formula 1 may be represented by one selected from Formula 3-1 to Formula 3-3:

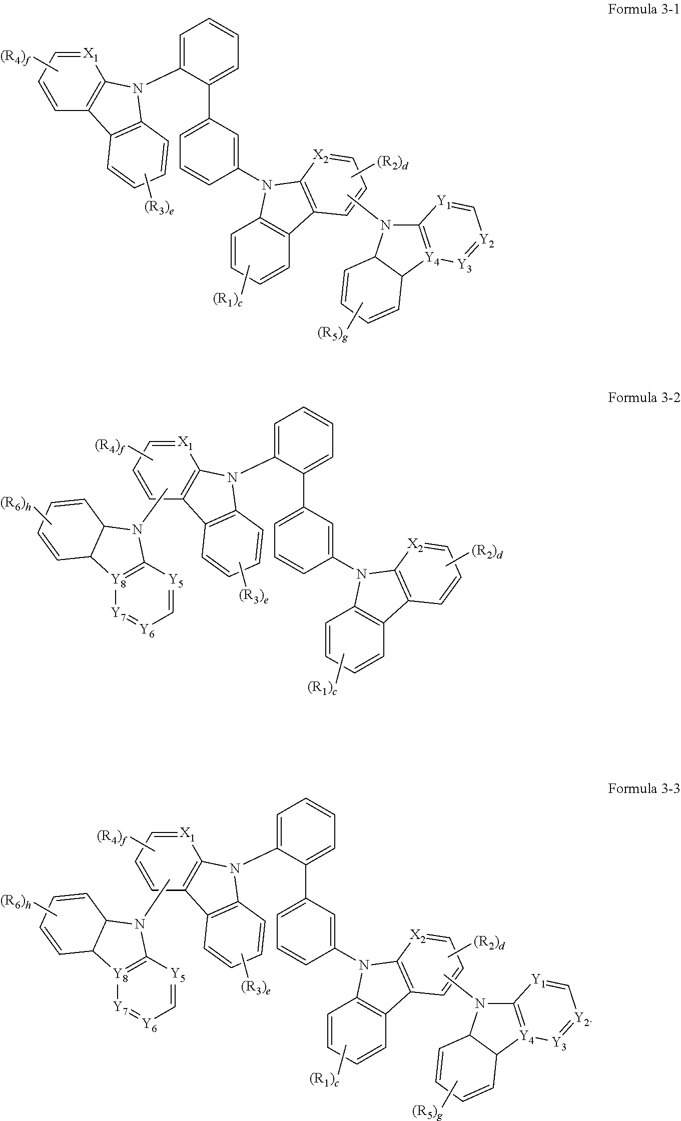

In Formula 3-1 to Formula 3-3, X₁, X₂, R₁ to R₆, Y₁ to Y₈, and “c” to “h” may each independently be the same as defined in Formula 1.

In an embodiment, the polycyclic compound represented by Formula 1 may be a blue light-emitting material.

In an embodiment, the polycyclic compound may be to emit phosphorescent light or thermally activated delayed fluorescent light.

In an embodiment of the present disclosure, a polycyclic compound is represented by Formula 1.

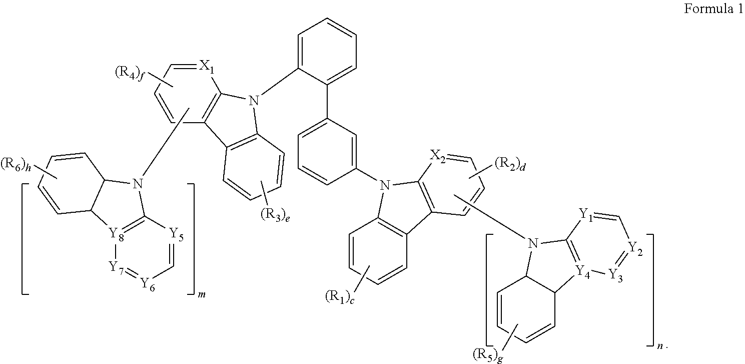

In Formula 1, “m” and “n” are each independently 0 or 1, “m+n” is 1 or more, X₁ and X₂ are each independently CRₐ or N, Y₁ to Y₈ are each independently CR_{b} or N, Rₐ, R_{b}, and R₁ to R₆ are each independently a hydrogen atom, a deuterium atom, a halogen atom, a cyano group, a substituted or unsubstituted silyl group, a substituted or unsubstituted alkyl group having 1 to 10 carbon atoms, a substituted or unsubstituted ring-forming aryl group having 6 to 30 carbon atoms, or a substituted or unsubstituted ring-forming heteroaryl group having 2 to 30 carbon atoms, “c”, “e”, “g”, and “h” are each independently an integer of 0 to 4, and “d” and “f” are each independently an integer of 0 to 3.

In an embodiment, the polycyclic compound represented by Formula 1 may be represented by Formula 2-1 or Formula 2-2.

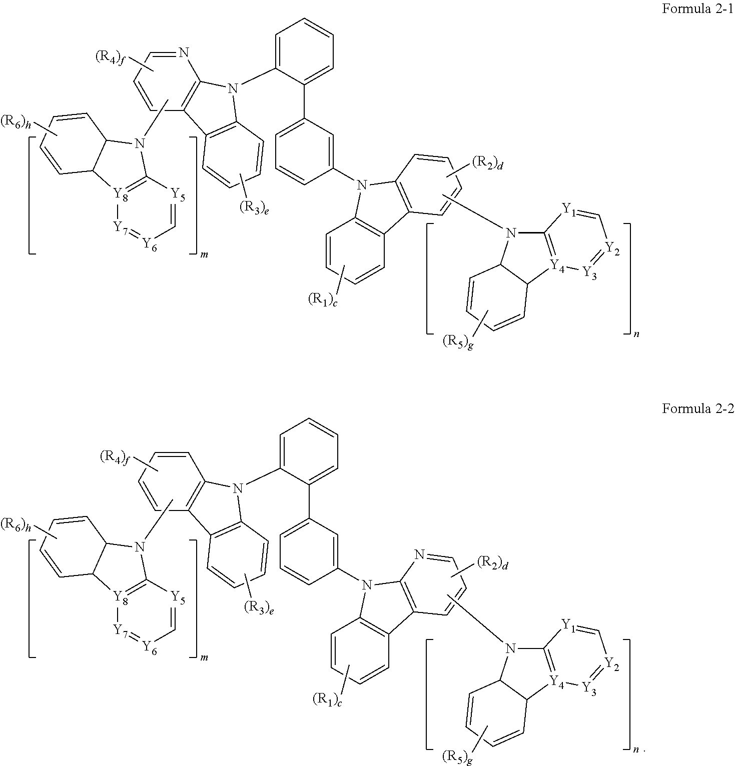

In Formula 2-1 and Formula 2-2, “m”, “n”, R₁ to R₆, “O” to “h”, and Y₁ to Y₈ may each independently be the same as defined in Formula 1.

In an embodiment, the polycyclic compound represented by Formula 1 may be represented by one selected from Formula 3-1 to Formula 3-3:

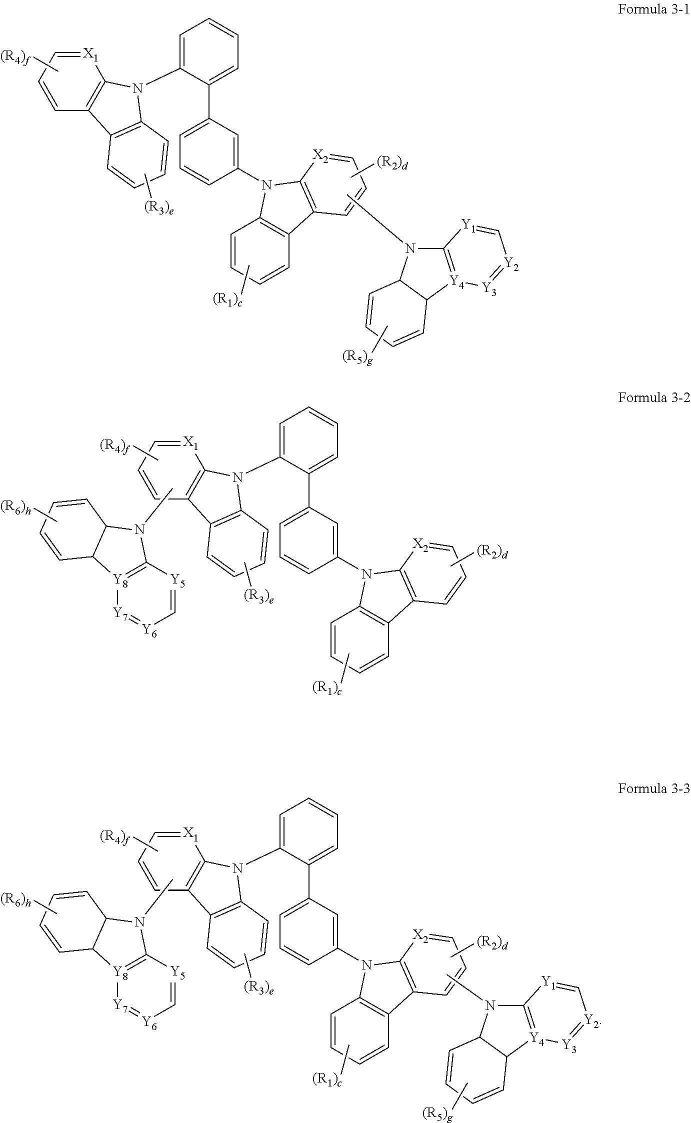

In Formula 3-1 to Formula 3-3, “c” to “h”, X₁, X₂, R₁ to R₆, and Y₁ to Y₈ may each independently be the same as defined in Formula 1.

In an embodiment, “c” to “h” may each be 1 or more, at least one of the R₁ to R₆ may be a deuterium atom.

In an embodiment, the polycyclic compound may be a blue light-emitting material.

### BRIEF DESCRIPTION OF THE DRAWINGS

The accompanying drawings are included to provide a further understanding of the present disclosure, and are incorporated in and constitute a part of this specification. The drawings illustrate example embodiments of the present disclosure and, together with the description, serve to explain principles of the present disclosure. In the drawings:

FIG. 1 is a cross-sectional view schematically illustrating an organic electroluminescent element according to an embodiment of the present disclosure;

FIG. 2 is a cross-sectional view schematically illustrating an organic electroluminescent element according to an embodiment of the present disclosure;

FIG. 3 is a cross-sectional view schematically illustrating an organic electroluminescent element according to an embodiment of the present disclosure; and

FIG. 4 is a cross-sectional view schematically illustrating an organic electroluminescent element according to an embodiment of the present disclosure.

### DETAILED DESCRIPTION

The present disclosure may have various modifications and may be embodied in different forms, and certain example embodiments will be described in detail with reference to the accompanying drawings. It should be understood, however, that the present disclosure is not intended to be limited to the example embodiments described herein, but includes all modifications, equivalents, and alternatives included within the spirit and scope of the present disclosure.

In describing each drawing, like reference numerals refer to like elements throughout, and duplicative descriptions thereof may not be provided. In the accompanying drawings, the dimensions of components may be shown in enlarged scale for clarity of illustration.

The terms first, second, etc. may be used herein to describe various components, and these components are not limited by these terms. These terms are used only to distinguish one component from another. For example, without departing from the scope of the present disclosure, a first component may be alternatively referred to as a second component, and similarly, a second component may be alternatively referred to as a first component. The singular forms, “a”, “an” and “the” are intended to include the plural forms as well, unless the context clearly indicates otherwise.

It should be understood that in the application, the terms “includes,” “including,” “comprises,” “comprising,” “have”, and/or the like are intended to indicate the presence of features, numerals, steps, operations, components, parts, or the combination thereof described herein, but do not preclude the presence or addition of one or more of other features, numerals, steps, operations, components, parts, or the combination thereof.

As used herein, expressions such as “at least one of,” “one of,” and “selected from,” when preceding a list of elements, modify the entire list of elements and do not modify the individual elements of the list. As used herein, the term “and/or” includes any and all combinations of one or more of the associated listed items.

Further, the use of “may” when describing embodiments of the present disclosure refers to “one or more embodiments of the present disclosure”.

In the application, when a part such as a layer, a film, a region, a plate is referred to as being “on” or “above” the other part, it may be “directly on” the other part, or an intervening part may also be present. In contrast, when an element is referred to as being “directly on,” or “directly above,” another element, etc., there are no intervening elements present. When a part such as a layer, a film, a region, a plate is referred to as being “under” or “below” the other part, it may be “directly under” the other part, or an intervening part may also be present. When a part is referred to as being disposed “on” the other part, it may be disposed on the upper part, or the lower part as well.

In the description, the term “substituted or unsubstituted” indicates that a group may be unsubstituted, or substituted with at least one substituent selected from the group consisting of a deuterium atom, a halogen atom, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, an alkoxy group, a hydrocarbon ring group, an aryl group, and a heterocyclic group. In some embodiments, each of the listed substituents may be further substituted or unsubstituted. For example, a biphenyl group may be interpreted as a named aryl group, or as a phenyl group substituted with a phenyl group.

In the description, the halogen atom may be a fluorine atom, a chlorine atom, a bromine atom, or an iodine atom.

In the description, the alkyl group may be a linear, branched, or cyclic group. The carbon number of the alkyl group may be 1 to 50, 1 to 30, 1 to 20, 1 to 10, or 1 to 6. Examples of the alkyl group include, but are not limited to, methyl, ethyl, n-propyl, isopropyl, n-butyl, s-butyl, t-butyl, i-butyl, 2-ethylbutyl, 3,3-dimethylbutyl, n-pentyl, i-pentyl, neopentyl, t-pentyl, cyclopentyl, 1-methylpentyl, 3-methylpentyl, 2-ethylpentyl, 4-methyl-2-pentyl, n-hexyl, 1-methylhexyl, 2-ethylhexyl, 2-butylhexyl, cyclohexyl, 4-methylcyclohexyl, 4-t-butylcyclohexyl, n-heptyl, 1-methylheptyl, 2,2-dimethylheptyl, 2-ethylheptyl, 2-butylheptyl, n-octyl, t-octyl, 2-ethyloctyl, 2-butyloctyl, 2-hexyloctyl, 3,7-dimethyloctyl, cyclooctyl, n-nonyl, n-decyl, adamantyl, 2-ethyldecyl, 2-butyldecyl, 2-hexyldecyl, 2-octyldecyl, n-undecyl, n-dodecyl, 2-ethyldodecyl, 2-butyldodecyl, 2-hexyldocecyl, 2-octyldodecyl, n-tridecyl, n-tetradecyl, n-pentadecyl, n-hexadecyl, 2-ethylhexadecyl, 2-butylhexadecyl, 2-hexylhexadecyl, 2-octylhexadecyl, n-heptadecyl, n-octadecyl, n-nonadecyl, n-eicosyl, 2-ethyleicosyl, 2-butyleicosyl, 2-hexyleicosyl, 2-octyleicosyl, n-henicosyl, n-docosyl, n-tricosyl, n-tetracosyl, n-pentacosyl, n-hexacosyl, n-heptacosyl, n-octacosyl, n-nonacosyl, n-triacontyl, etc.

In the description, the term “aryl group” refers to an optional functional group or a substituent derived from an aromatic hydrocarbon ring. The aryl group may be a monocyclic aryl group or a polycyclic aryl group. The number of ring-forming carbon atoms of the aryl group may be 6 to 30, 6 to 20, or 6 to 15. Examples of the aryl group include, but are not limited to, phenyl, naphthyl, fluorenyl, anthracenyl, phenanthryl, biphenyl, terphenyl, quaterphenyl, quinquephenyl, sexiphenyl, triphenylenyl, pyrenyl, benzofluoranthenyl, chrysenyl, etc.

In the description, the fluorenyl group may be substituted (e.g., at the 9H position), and two substituents may be combined with each other to form a spiro structure. Examples of the substituted fluorenyl group are as follows, but are not limited thereto:

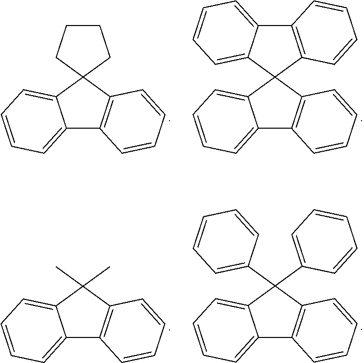

In the description, the heteroaryl group may include one or more among boron (B), oxygen (O), nitrogen (N), phosphorus (P), silicon (Si), and sulfur (S) as a heteroatom. When the heteroaryl group includes two or more heteroatoms, the two or more heteroatoms may be the same or different from each other. The heteroaryl group may be a monocyclic heteroaryl group or a polycyclic heteroaryl group. The number of ring-forming carbon atoms of the heteroaryl group may be 2 to 30, 2 to 20, or 2 to 10. Examples of the heteroaryl group include, but are not limited to thiophene, furan, pyrrole, imidazole, triazole, pyridine, bipyridine, pyrimidine, triazine, triazole, acridyl, pyridazine, pyrazinyl, quinoline, quinazoline, quinoxaline, phenoxazine, phthalazine, pyrido pyrimidine, pyrido pyrazine, pyrazino pyrazine, isoquinoline, indole, carbazole, N-arylcarbazole, N-heteroarylcarbazole, N-alkylcarbazole, benzoxazole, benzimidazole, benzothiazole, benzocarbazole, benzothiophene, dibenzothiophene, thienothiophene, benzofuran, phenanthroline, thiazole, isoxazole, oxazole, oxadiazole, thiadiazole, phenothiazine, dibenzosilole, dibenzofuran, etc.

In the description, the term “silyl group” includes an alkyl silyl group and an aryl silyl group. Examples of the silyl group include, but are not limited to trimethylsilyl, triethylsilyl, t-butyldimethylsilyl, vinyldimethylsilyl, propyldimethylsilyl, triphenylsilyl, diphenylsilyl, phenylsilyl, etc.

In the description, the term “direct linkage” may refer to a single bond.

In the description, “ [symbol] .” indicates a position to be connected (e.g., to another group or moiety).

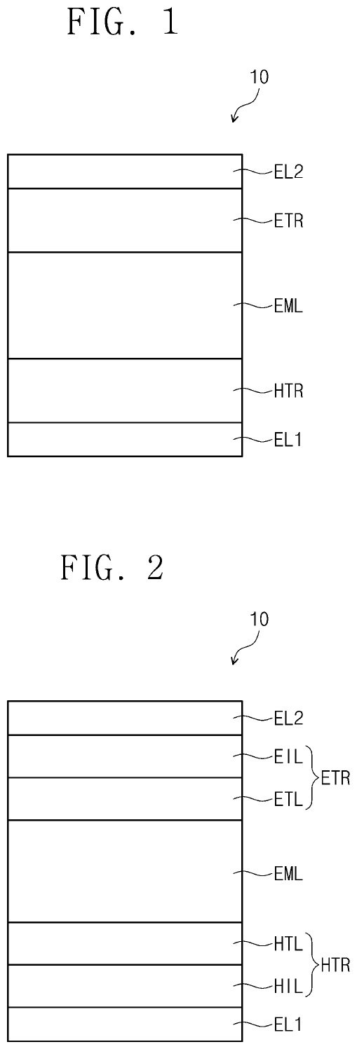

FIG. 1 is a cross-sectional view schematically illustrating an organic electroluminescent element according to an embodiment of the present disclosure. An organic electroluminescent element 10 according to an embodiment may include a first electrode EL1, a hole transport region HTR, an emission layer EML, an electron transport region ETR, and a second electrode EL2 stacked in order.

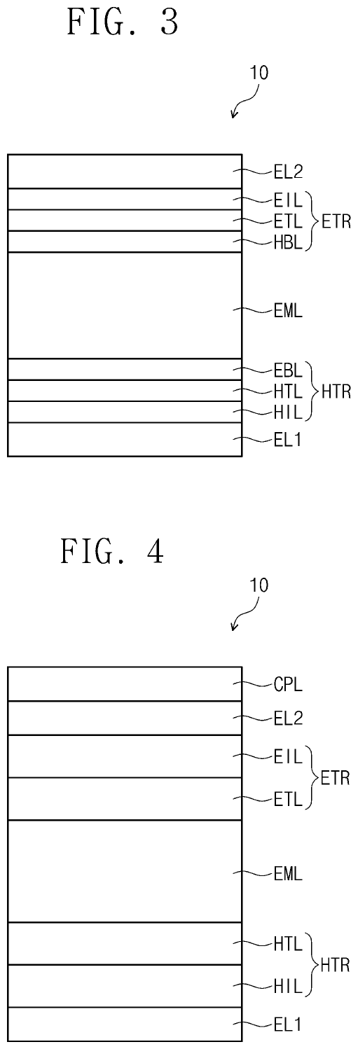

Compared with FIG. 1, FIG. 2 shows the cross-sectional view of an organic electroluminescent element 10 of an embodiment, in which the hole transport region HTR includes a hole injection layer HIL and a hole transport layer HTL, and the electron transport region ETR includes an electron injection layer EIL and an electron transport layer ETL. Compared with FIG. 1, FIG. 3 shows the cross-sectional view of an organic electroluminescent element 10 of an embodiment, in which the hole transport region HTR includes the hole injection layer HIL, the hole transport layers HTL, and an electron blocking layer EBL, and the electron transport region ETR includes the electron injection layer EIL, the electron transport layer ETL, and a hole blocking layer HBL.

FIG. 1 to FIG. 4 are cross-sectional views schematically showing organic electroluminescent elements according to embodiments of the present disclosure. Referring to FIG. 1 to FIG. 4, in each of the organic electroluminescent elements 10, a first electrode EL1 and a second electrode EL2 are disposed to face each other, and an emission layer EML is between the first electrode EL1 and the second electrode EL2.

In some embodiments, the organic electroluminescent elements 10 may further include a plurality of functional layers between the first electrode EU and the second electrode EL2 in addition to the emission layer EML. The plurality of the functional layers may include a hole transport region HTR and an electron transport region ETR. For example, an organic electroluminescent element 10 of an embodiment may include a first electrode EL1, a hole transport region HTR, an emission layer EML, an electron transport region ETR, and a second electrode EL2, stacked in this order. In some embodiments, an organic electroluminescent element 10 of an embodiment may include a capping layer CPL disposed on the second electrode EL2.

An organic electroluminescent element 10 of an embodiment may include a polycyclic compound of an embodiment (described below) in an emission layer EML disposed between a first electrode EU and a second electrode EL2. However, an embodiment of the present disclosure is not limited thereto. In some embodiments, the organic electroluminescent element 10 of an embodiment may include the polycyclic compound in at least one of a hole transport region HTR and an electron transport region ETR, which are among the plurality of functional layers disposed between the first electrode EL1 and the second electrode EL2 in addition to the emission layer EML, or in some embodiments the polycyclic compound according to an embodiment may be included in a capping layer CPL disposed on the second electrode EL2.

Compared with FIG. 1, FIG. 2 shows the cross-sectional view of an organic electroluminescent element 10 of an embodiment, wherein the hole transport region HTR includes a hole injection layer HIL and a hole transport layer HTL, and the electron transport region ETR includes an electron injection layer EIL and an electron transport layer ETL. Compared with FIG. 1, FIG. 3 shows the cross-sectional view of an organic electroluminescent element 10 of an embodiment, wherein the hole transport region HTR includes the hole injection layer HIL, the hole transport layer HTL, and an electron blocking layer EBL, and the electron transport region ETR includes the electron injection layer EIL, the electron transport layer ETL, and a hole blocking layer HBL. Compared with FIG. 2, FIG. 4 shows the cross-sectional view of an organic electroluminescent element 10 of an embodiment, including a capping layer CPL disposed on a second electrode EL2.

The first electrode EL1 may have conductivity (e.g., may be conductive). The first electrode EL1 may be formed using a metal alloy or a conductive compound. The first electrode EL1 may be an anode. In some embodiments, the first electrode EL1 may be a pixel electrode. The first electrode EL1 may be a transmissive electrode, a transflective electrode, or a reflective electrode. When the first electrode EL1 is a transmissive electrode, the first electrode EU may include a transparent metal oxide, for example, indium tin oxide (ITO), indium zinc oxide (IZO), zinc oxide (ZnO), indium tin zinc oxide (ITZO), etc. If the first electrode EL1 is the transflective electrode or the reflective electrode, the first electrode EU may include Ag, Mg, Cu, Al, Pt, Pd, Au, Ni, Nd, Ir, Cr, Li, Ca, LiF/Ca, LiF/Al, Mo, Ti, or a compound thereof, or a mixture thereof (for example, a mixture of Ag and Mg). In some embodiments, the first electrode EL1 may have a structure including a plurality of layers including a reflective layer or a transflective layer formed using the materials above, and a transmissive conductive layer formed using indium tin oxide (ITO), indium zinc oxide (IZO), zinc oxide (ZnO), indium tin zinc oxide (ITZO), etc. For example, the first electrode EL1 may have a three-layer structure of ITO/Ag/ITO, but is not limited thereto. The thickness of the first electrode EL1 may be about 1,000 Å to about 10,000 Å, for example, about 1,000 Å to about 3,000 Å.

A hole transport region HTR is provided on the first electrode EL1. The hole transport region HTR may include at least one of a hole injection layer HIL, a hole transport layer HTL, a hole buffer layer, or an electron blocking layer EBL. The thickness of the hole transport region HTR may be, for example, about 50 Å to about 15,000 Å.

The hole transport region HTR may have a single layer structure formed using a single material, a single layer structure formed using a plurality of different materials, or a multilayer structure including a plurality of layers formed using a plurality of different materials.

For example, the hole transport region HTR may have a single layer structure of a hole injection layer HIL or a hole transport layer HTL, or may have a single layer structure formed using a hole injection material and a hole transport material. In some embodiments, the hole transport region HTR may have a single layer structure formed using a plurality of different materials, or a hole injection layer HIL/hole transport layer HTL, hole injection layer HIL/hole transport layer HTL/hole buffer layer, hole injection layer HIL/hole buffer layer, hole transport layer HTL/hole buffer layer, or hole injection layer HIL/hole transport layer HTL/electron blocking layer EBL structure sequentially stacked from the first electrode EL1. However, embodiments of the present disclosure are not limited thereto.

The hole transport region HTR may be formed using any suitable method (such as a vacuum deposition method, a spin coating method, a cast method, a Langmuir-Blodgett (LB) method, an inkjet printing method, a laser printing method, and/or a Laser Induced Thermal Imaging (LITI) method).

The hole injection layer HIL may include, for example, a phthalocyanine compound (such as copper phthalocyanine), N1,N1′-([1,1′-biphenyl]-4,4′-diyl)bis(N1-phenyl-N4,N4-di-m-tolylbenzene-1,4-diamine) (DNTPD), 4,4′,4″-[tris(3-methylphenyl)phenylamino] triphenylamine (m-MTDATA), 4,4′,4″-tris(N,N-diphenylamino)triphenylamine (TDATA), 4,4′,4″-tris[N(2-naphthyl)-N-phenylamino]-triphenylamine (2-TNATA), poly(3,4-ethylene dioxythiophene)/poly(4-styrene sulfonate) (PEDOT/PSS), polyaniline/dodecylbenzene sulfonic acid (PANI/DBSA), polyaniline/camphor sulfonic acid (PANI/CSA), polyaniline/poly(4-styrene sulfonate) (PAN I/PSS), N,N′-di(naphthalene-1-yl)-N,N′-diphenyl-benzidine (NPB), triphenylamine-containing polyether ketone (TPAPEK), 4-isopropyl-4′-methyldiphenyliodonium [tetrakis(pentafluorophenyl)borate], dipyrazino[2,3-f:2′,3′-h]quinoxaline-2,3,6,7,10,11-hexacarbonitrile (HAT-CN), etc.

The hole transport layer HTL may further include, for example, a carbazole-based derivative (such as N-phenyl carbazole and/or polyvinyl carbazole), a fluorine-based derivative, a triphenylamine-based derivative (such as N,N′-bis(3-methylphenyl)-N,N′-diphenyl-[1,1-biphenyl]-4,4′-diamine (TPD)), 4,4′,4″-tris(N-carbazolyl)triphenylamine (TCTA), N,N′-di(naphthalene-1-yl)-N,N′-diphenyl-benzidine (NPB), 4,4′-cyclohexylidene bis[N,N-bis(4-methylphenyl)benzenamine] (TAPC), 4,4′-bis[N,N′-(3-tolyl)amino]-3,3′-dimethylbiphenyl (HMTPD), 1,3-bis(N-carbazolyl)benzene (mCP), etc.

The electron blocking layer EBL may include, for example, a carbazole-based derivative (such as N-phenyl carbazole and/or polyvinyl carbazole), a fluorene-based derivative, a triphenylamine-based derivative (such as N,N′-bis(3-methylphenyl)-N,N′-diphenyl-[1,1′-biphenyl]-4,4′-diamine (TPD)), 4,4′,4″-tris(N-carbazolyl)triphenylamine (TCTA), N,N′-di(1-naphthalene-1-yl)-N,N′-diphenyl-benzidine (NPB), 4,4′-cyclohexylidene bis[N,N-bis(4-methylphenyl)benzeneamine (TAPC), 4,4′-bis[N,N′-(3-tolyl)amino]-3,3′-dimethylbiphenyl (HMTPD), 9-(4-tert-butylphenyl)-3,6-bis(triphenylsilyl)-9H-carbazole (CzSi), 9-phenyl-9H-3,9′-bicarbazole (CCP), 1,3-bis(N-carbazolyl)benzene (mCP), 1,3-bis(1,8-dimethyl-9H-carbazol-9-yl)benzene (mDCP), or so on.

The thickness of the hole transport region HTR may be about 100 Å to about 10,000 Å, for example, about 100 Å to about 5,000 Å. The thickness of the hole injection layer HIL may be, for example, about 30 Å to about 1,000 Å, and the thickness of the hole transport layer HTL may be about 30 Å to about 1,000 Å. For example, the thickness of the electron blocking layer EBL may be about 10 Å to about 1,000 Å. When the thicknesses of the hole transport region HTR, the hole injection layer HIL, the hole transport layer HTL and the electron blocking layer EBL satisfy the above-described ranges, satisfactory hole transport characteristics may be achieved without substantial increase of a driving voltage.

The hole transport region HTR may further include a charge-generating material in addition to the above-described materials to increase conductivity. The charge-generating material may be dispersed substantially uniformly or non-uniformly in the hole transport region HTR. The charge-generating material may be, for example, a p-dopant. The p-dopant may be one of a quinone derivative, a metal oxide, or a cyano group-containing compound, but is not limited thereto. For example, non-limiting examples of the p-dopant include a quinone derivative (such as tetracyanoquinodimethane (TCNQ) and/or 2,3,5,6-tetrafluoro-7,7′,8,8-tetracyanoquinodimethane (F4-TCNQ)), and a metal oxide (such as a tungsten oxide and/or a molybdenum oxide).

As described above, the hole transport region HTR may further include at least one of a hole buffer layer or the electron blocking layer EBL in addition to the hole injection layer HIL and the hole transport layer HTL. The hole buffer layer may compensate for a resonance distance of the wavelength of light emitted from the emission layer EML to increase luminous efficiency. Materials that may be included in the hole transport region HTR may also be included in the hole buffer layer. The electron blocking layer EBL may prevent or reduce electron injection from the electron transport region ETR to the hole transport region HTR.

The emission layer EML is provided on the hole transport region HTR. The emission layer EML may have a thickness of, for example, about 100 Å to about 1,000 Å or about 100 Å to about 300 Å. The emission layer EML may have a single layer structure formed using a single material, a single layer structure formed using a plurality of different materials, or a multilayer structure having a plurality of layers formed using a plurality of different materials.

The emission layer EML may include a polycyclic compound of an embodiment. The polycyclic compound of an embodiment includes a first benzene ring and a second benzene ring, which are linked by a single bond, a first carbazole group substituted in an ortho position of the first benzene ring with respect to the single bond, a second carbazole group substituted in a meta position of the second benzene ring with respect to the single bond, and a third carbazole group substituted on at least one of the first carbazole group or the second carbazole group. In other words, the first benzene ring and the second benzene ring linked by a single bond may be a biphenyl structure. The first carbazole group and the second carbazole group may be opposite to each other with respect to the single bond as an axis (e.g., when the polycyclic compound is in an energetically stable conformation, the first carbazole group and the second carbazole group may be on opposite sides of the compound with respect to the central axis of the biphenyl group single bond). However, embodiments of the present disclosure are not limited thereto.

At least one third carbazole group may be substituted on the first carbazole group or the second carbazole group. For example, the third carbazole group may be substituted only in the first carbazole group, only in the second carbazole group, or in both the first carbazole group and the second carbazole group (e.g., simultaneously). The third carbazole group respectively substituted in the first carbazole group and the second carbazole group may be the same or different from each other.

An emission layer EML of an organic electroluminescent element 10 of an embodiment may include a polycyclic compound of an embodiment, represented by Formula 1:

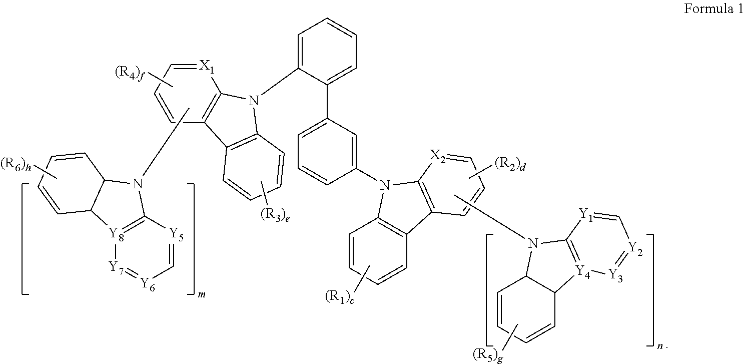

In Formula 1, “m” and “n” may each independently 0 or 1, and “m+n” may be 1 or more (e.g., at least one of m and n is 1). For example, both “m” and “n” may be 1 (e.g., simultaneously), or at least one of “m” or “n” may be 0, and the other may be 1.

X₁ and X₂ may each independently be CRₐ or N. Both X₁ and X₂ may be CRₐ, both X₁ and X₂ may be N, or at least one of X₁ or X₂ may be N, and the other may be CRₐ.

Y₁ to Y₈ may each independently be CR_{b} or N. All of Y₁ to Y₈ may be CR_{b}, at least one among Y₁ to Y₄ may be N and all of Y₅ to Y₈ may be CR_{b}, all of Y₁ to Y₄ may be CR_{b} and at least one among Y₅ to Y₈ may be N, or at least one among Y₁ to Y₄ may be N and at least one among Y₅ to Y₈ may be N.

In formula 1, “c”, “e”, “g”, and “h” may each independently be an integer of 0 to 4, and “d” and “f” may each independently be an integer of 0 to 3.

Hereinafter, a carbazole group included in a polycyclic compound represented by Formula 1 is defined as a first carbazole group when the carbazole group is substituted in an ortho position of the first benzene ring with respect to the single bond of the biphenyl group, a second carbazole group when the carbazole group is substituted in an meta position of the second benzene ring with respect to the single bond of the biphenyl group, and a third carbazole group when the carbazole group is additionally substituted on a carbazole group bonded to the biphenyl group (e.g., on the first or second carbazole group).

The first carbazole group and the second carbazole group may each independently be an unsubstituted carbazole group or a carbazole group including 1 to 8 substituents. The third carbazole group(s) may each independently be an unsubstituted carbazole group or a carbazole group including 1 to 4 substituents. For example, in a polycyclic compound, “c” to “h” may each be 1 or more, and at least one of R₁ to R₆ may be a deuterium atom. However, embodiments of the present disclosure are not limited thereto.

Rₐ, R_{b}, and R₁ to R₆ may each independently be a hydrogen atom, a deuterium atom, a halogen atom, a cyano group, a substituted or unsubstituted silyl group, a substituted or unsubstituted alkyl group having 1 to 10 carbon atoms, a substituted or unsubstituted ring-forming aryl group having 6 to 30 carbon atoms, or a substituted or unsubstituted ring-forming heteroaryl group having 2 to 30 carbon atoms. However, embodiments of the present disclosure are not limited thereto.

The first carbazole group to the third carbazole group may each independently be represented by one selected from Formula C1 to Formula C10:

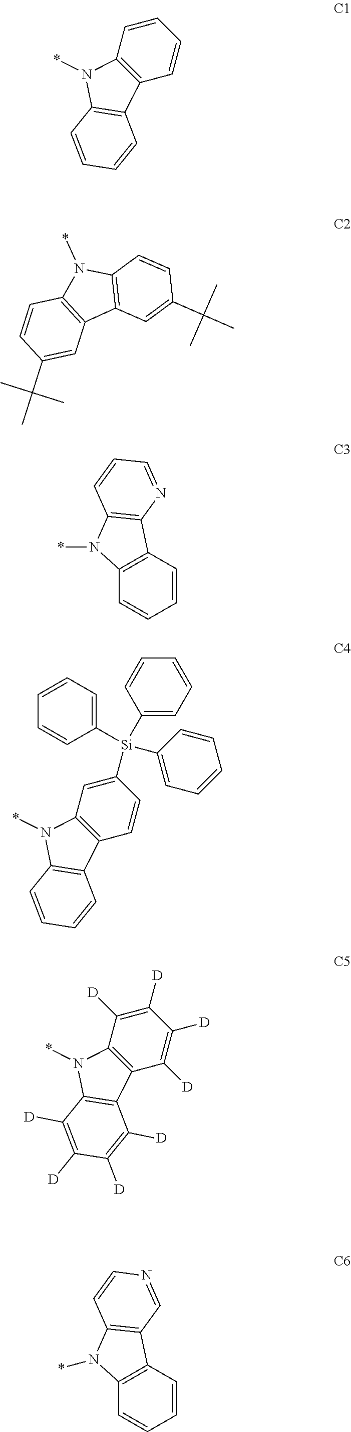

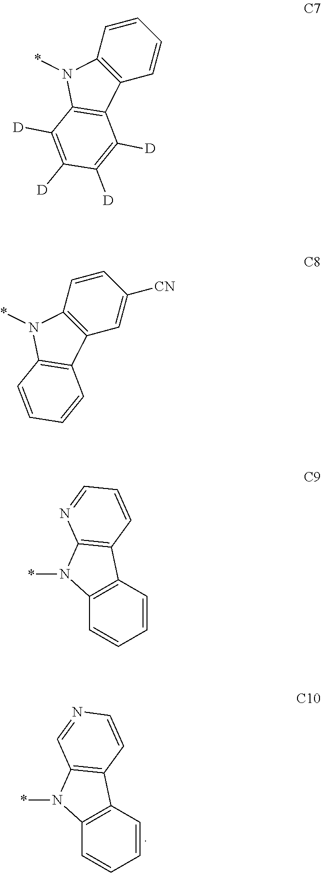 [SMILES: *n1c2ccccc2c2c([2H])c([2H])c([2H])c([2H])c21 ; *n1c2ccccc2c2cc(C#N)ccc21 ; *n1c2ccccc2c2cccnc21 ; *n1c2ccccc2c2ccncc21]

The polycyclic compound of an embodiment represented by Formula 1 may be represented by one selected from Formula 2-1 to Formula 2-3. Formula 2-1 to Formula 2-3 represent examples (structures) of Formula 1 in which the type of atom at the 1-carbon site of the first carbazole group and the second carbazole group are specified. For example, Formula 2-1 represents a case in which both X₁ of the first carbazole group and X₂ of the second carbazole group are CRₐ in Formula 1, Formula 2-2 represents a case in which X₁ of the first carbazole group is N and X₂ of the second carbazole group is CRₐ in Formula 1, and Formula 2-3 represents a case in which X₁ of the first carbazole group is CRₐ and X₂ of the second carbazole group are both N in Formula 1.

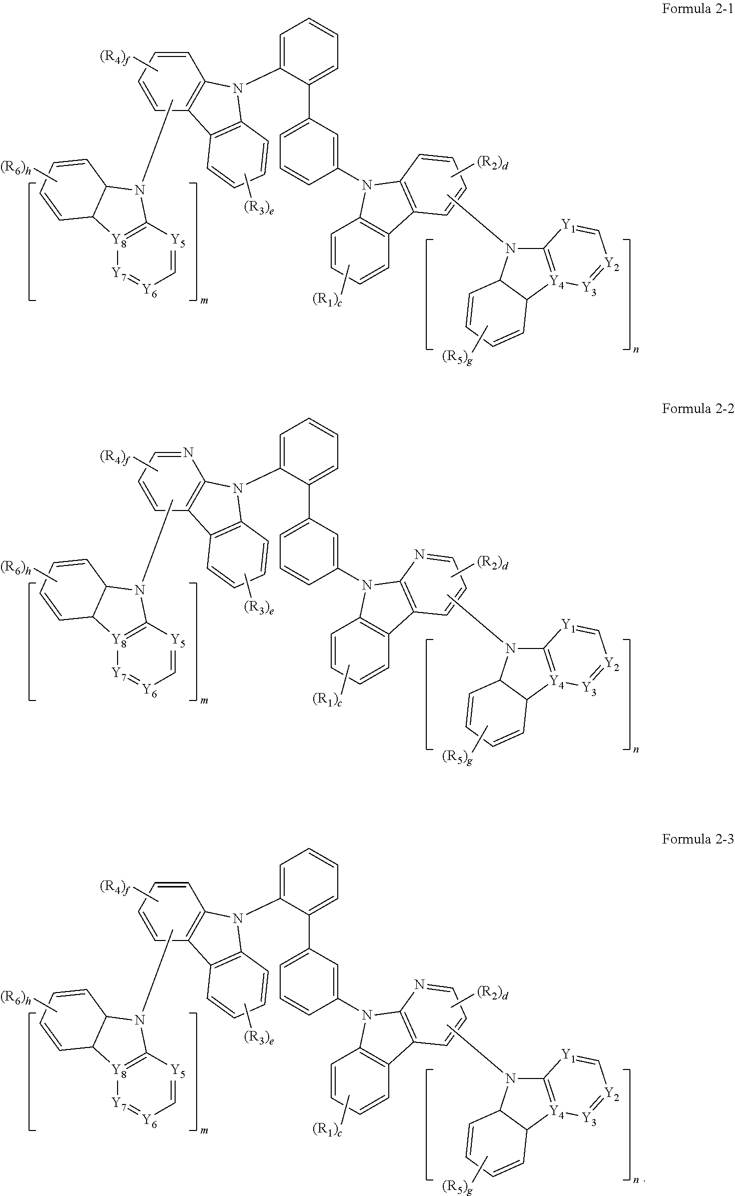

In Formula 2-1 to Formula 2-3, R₁ to R₆, “O^{”} to “h^{”}, “m” to “n” and Y₁ to Y₈ may each independently be the same as described in Formula 1.

A polycyclic compound of an embodiment represented by Formula 1 may be represented by one selected from Formula 3-1 to Formula 3-3. Formula 3-1 to Formula 3-3 represent examples (structures) of Formula 1 in which the substitution position(s) of the third carbazole group (e.g., on at least one of the first carbazole group or the second carbazole group) are specified. For example, Formula 3-1 to Formula 3-3 are compounds of Formula 1 in which the third carbazole group is substituted on the first carbazole group, the third carbazole group is substituted on the second carbazole group, or the third carbazole group is substituted on each of the first carbazole group and the second carbazole group (e.g., a third carbazole group is substituted on the first carbazole group, and a fourth carbazole group is substituted on the second carbazole group).

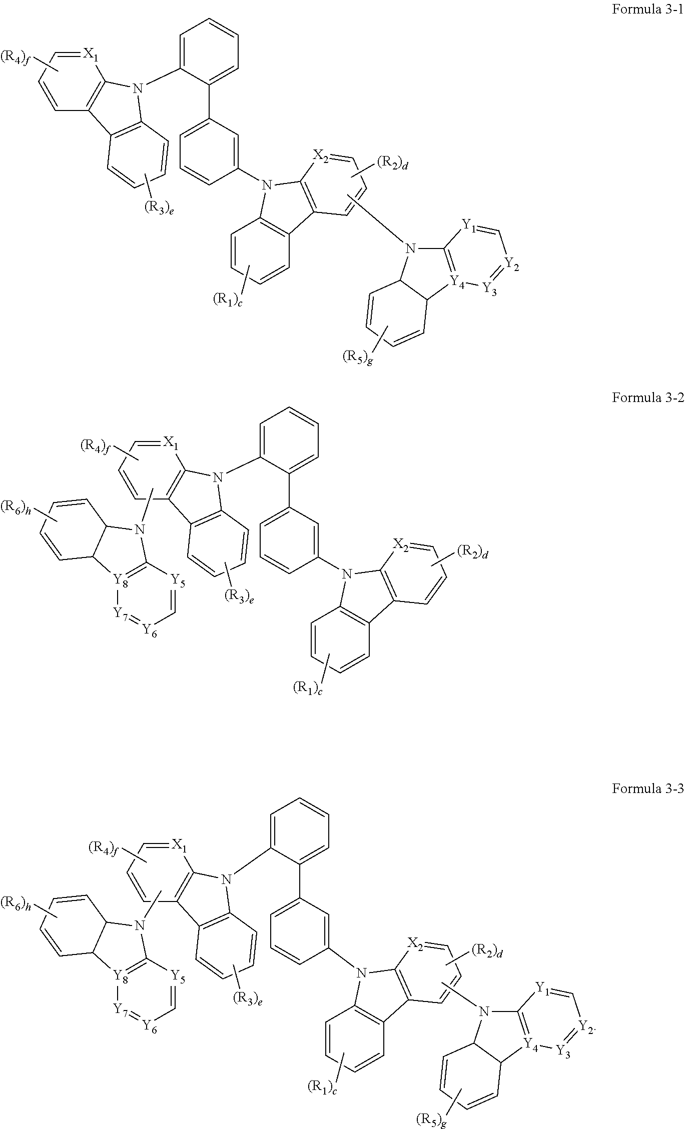

In an embodiment, a polycyclic compound represented by Formula 1 may be used as a blue light-emitting material.

In an embodiment, the aforementioned polycyclic compound may be used as a host. For example, the organic electroluminescent element 10 of an embodiment may include a host and a dopant, and the host may include the aforementioned polycyclic compound of an embodiment.

In an organic electroluminescent element 10 of an embodiment, the emission layer may be to emit blue light.

In an embodiment, the emission layer EML may further include any suitable material in the art as a host material in addition to the polycyclic compound of an embodiment. For example, the emission layer EML may include, as a host material, at least one of bis[2-(diphenylphosphino)phenyl] ether oxide (DPEPO), 4,4′-bis(carbazol-9-yl)biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), or 1,3,5-tris(1-phenyl-1H-benzo[d]imidazole-2-yl)benzene (TPBi), but is not limited thereto. For example, tris(8-hydroxyquinolino)aluminum (Alq₃), 4,4′-bis(N-carbazolyI)-1,1′-biphenyl (CBP), poly(N-vinylcarbazole) (PVK), 9,10-di(naphthalene-2-yl)anthracene (ADN), 4,4′,4″-tris(carbazol-9-yl)-triphenylamine (TCTA), 1,3,5-tris(N-phenylbenzimidazole-2-yl)benzene (TPBi), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), 2,8-bis(diphenylphosphoryl)dibenzofuran (PPF), etc. may be used as a host material.

In some embodiments, the emission layer EML may further include any suitable material in the art as a dopant material in addition to the polycyclic compound of an embodiment. For example, the emission layer EML may include, as a dopant material, styryl derivatives (for example, 1,4-bis[2-(3-N-ethylcarbazolyl)vinyl]benzene (BCzVB), 4-(di-p-tolylamino)-4′-[(di-p-tolylamino)styryl]stilbene (DPAVB), and N-(4-((E)-2-(6-((E)-4-(diphenylamino)styryl)naphthalen-2-yl)vinyl)phenyl)-N-phenylbenz enamine (N-BDAVBi)), perylene and derivatives thereof (for example, 2,5,8,11-tetra-t-butylperylene (TBP)), pyrene and derivatives thereof (for example, 1,1-dipyrene, 1,4-dipyrenylbenzene, 1,4-bis(N,N-diphenylamino)pyrene), etc.

The emission layer EML may further include any suitable phosphorescent dopant material. For example, a metal complex including iridium (Ir), platinum (Pt), osmium (Os), gold (Au), titanium (Ti), zirconium (Zr), hafnium (Hf), europium (Eu), terbium (Tb), or thulium (Tm) may be used as a phosphorescent dopant. For example, iridium (III) bis(4,6-difluorophenylpyridinato-N,C2) (FIrpic), bis(2,4-difluorophenylpyridinato)-tetrakis(1-pyrazolyl)borate iridium (III) (Fir6), or platinum octaethyl porphyrin (PtOEP) may be used as a phosphorescent dopant. However, embodiments of the present disclosure are not limited thereto.

In some embodiments, the emission layer EML may further include any suitable phosphorescent host material, for example, bis(4-(9H-carbazol-9-yl)phenyl)diphenylsilane (BCPDS).

When the emission layer EML is to emit blue light, the emission layer EML may further include a fluorescence (fluorescent) material including one selected from the group consisting of spiro-DPVBi, spiro-6P, distyryl-benzene (DSB), distyryl-arylene (DSA), a polyfluorene (PFO)-based polymer, and a poly(p-phenylene vinylene (PPV)-based polymer. When the emission layer EML is to emit blue light, a dopant included in the emission layer EML may be selected from, for example, an organometallic complex or a metal complex (such as (4,6-F2ppy)₂Irpic), and perylene and derivatives thereof.

In some embodiments, an organic electroluminescent element 10 of an embodiment may include a plurality of emission layers. The plurality of emission layers may be stacked and provided in the described in order. For example, an organic electroluminescent element 10 including a plurality of emission layers may be to emit white light. The organic electroluminescent element including a plurality of emission layers may be an organic electroluminescent element having a tandem structure. When the organic electroluminescent element 10 includes a plurality of emission layers, at least one emission layer EML may include the aforementioned polycyclic compound according to an embodiment.

In the organic electroluminescent elements 10 as shown in FIG. 1 to FIG. 4, an electron transport region ETR is provided on an emission layer EML. The electron transport region ETR may include at least one of a hole blocking layer HBL, an electron transport layer ETL, or an electron injection layer EIL, but embodiments of the present disclosure are not limited thereto.

The electron transport region ETR may have a single layer structure formed using a single material, a single layer structure formed using a plurality of different materials, or a multilayer structure having a plurality of layers formed using a plurality of different materials.

For example, the electron transport region ETR may have a single layer structure of an electron injection layer EIL or an electron transport layer ETL, or a single layer structure formed using an electron injection material and an electron transport material. In some embodiments, the electron transport region ETR may have a single layer structure formed using a plurality of different materials, or an electron transport layer ETL/electron injection layer EIL, or hole blocking layer HBL/electron transport layer ETL/electron injection layer EIL structure sequentially stacked from the emission layer EML, but is not limited thereto. The thickness of the electron transport region ETR may be, for example, about 1,000 Å to about 1,500 Å.

The electron transport region ETR may be formed using any suitable method (such as a vacuum deposition method, a spin coating method, a cast method, a Langmuir-Blodgett (LB) method, an inkjet printing method, a laser printing method, and/or a Laser Induced Thermal Imaging (LITI) method).

When the electron transport region ETR includes an electron transport layer ETL, the electron transport region ETR may include an anthracene-based compound. However, embodiments of the present disclosure are not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzimidazolyl-1-yl)phenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,08)-(1,1′-biphenyl-4-olato)aluminum (BAlq), beryllium bis(benzoquinolin-10-olate (Bebq2), 9,10-di(naphthalene-2-yl)anthracene (ADN), 1,3-bis[3,5-di(pyridine-3-yl)phenyl]benzene (BmPyPhB), and a mixture thereof. The thickness of the electron transport layers ETL may be about 100 Å to about 1,000 Å, for example, about 150 Å to about 500 Å. When the thickness of the electron transport layers ETL satisfies the above-described range, satisfactory electron transport characteristics may be obtained without substantial increase of a driving voltage.

When the electron transport region ETR includes an electron injection layer EIL, the electron transport region ETR may be formed of or may use a metal halide (such as LiF, NaCl, CsF, RbCl, RbI, and/or CuI), a lanthanide metal (such as Yb), a metal oxide (such as Li₂O and/or BaO), or 8-hydroxyl-lithium quinolate (LiQ). However, embodiments of the present disclosure are not limited thereto. The electron injection layer EIL may also be formed using a mixture material of an electron transport material and an insulating organo metal salt. The organo metal salt may have an energy band gap of about 4 eV or more. In some embodiments, the organo metal salt may include, for example, a metal acetate, a metal benzoate, a metal acetoacetate, a metal acetylacetonate, or a metal stearate. The thickness of the electron injection layer EIL may be about 1 Å to about 100 Å, or about 3 Å to about 90 Å. When the thickness of the electron injection layer EIL satisfies the above described range, satisfactory electron injection characteristics may be obtained without substantial increase in driving voltage.

The electron transport region ETR may include a hole blocking layer HBL as described above. The hole blocking layer HBL may include, for example, at least one of 2,9-dimethyl-4,7-diphenyl-1,10-phenanthroline (BCP) or 4,7-diphenyl-1,10-phenanthroline (Bphen). However, embodiments of the present disclosure are not limited thereto.

The second electrode EL2 is provided on the electron transport region ETR. The second electrode EL2 may be a common electrode or a cathode. The second electrode EL2 may be a transmissive electrode, a transflective electrode or a reflective electrode. When the second electrode EL2 is a transmissive electrode, the second electrode EL2 may be composed of a transparent metal oxide, for example, indium tin oxide (ITO), indium zinc oxide (IZO), zinc oxide (ZnO), indium tin zinc oxide (ITZO), etc.

When the second electrode EL2 is a transflective electrode or a reflective electrode, the second electrode EL2 may include silver (Ag), magnesium (Mg), copper (Cu), aluminum (Al), platinum (Pt), palladium (Pd), gold (Au), nickel (Ni), neodymium (Nd), iridium (Ir), chromium (Cr), lithium (Li), calcium (Ca), LiF/Ca, LiF/AI, molybdenum (Mo), titanium (Ti), ytterbium (Yb), =a compound thereof, or a mixture thereof (for example, AgMg, AgYb, or MgAg). In some embodiments, the second electrode EL2 may have a multilayered structure including a reflective layer or a transflective layer formed using the above-described materials and a transparent conductive layer formed using indium tin oxide (ITO), indium zinc oxide (IZO), zinc oxide (ZnO), indium tin zinc oxide (ITZO), etc.

In some embodiments, the second electrode EL2 may be connected to an auxiliary electrode. When the second electrode EL2 is connected with the auxiliary electrode, the resistance of the second electrode EL2 may decrease.

In some embodiments, a capping layer CPL may be further disposed on the second electrode EL2 of the organic electroluminescent element 10 of an embodiment. The capping layer CPL may include (e.g., be) multiple layers or a single layer.

In an embodiment, the capping layer CPL may be an organic layer or an inorganic layer. For example, when the capping layer CPL includes an inorganic material, the inorganic material may include an alkali metal compound such as LiF, an alkaline earth metal compound such as MgF₂, SiON, SiNₓ, SiO_{y}, etc.

For example, when the capping layer CPL includes an organic material, the organic material may include α-NPD, NPB, TPD, m-MTDATA, Alq₃, CuPc, N4,N4,N4′,N4′-tetra(biphenyl-4-yl) biphenyl-4,4′-diamine (TPD15), 4,4′,4″-tris(carbazol-9-yl) triphenylamine (TCTA), etc., an epoxy resin, or an acrylate (such as methacrylate). However, embodiments of the present disclosure are not limited thereto. The capping layer CPL may include one or more of Compound P1 to Compound P5 in addition to the aforementioned materials.

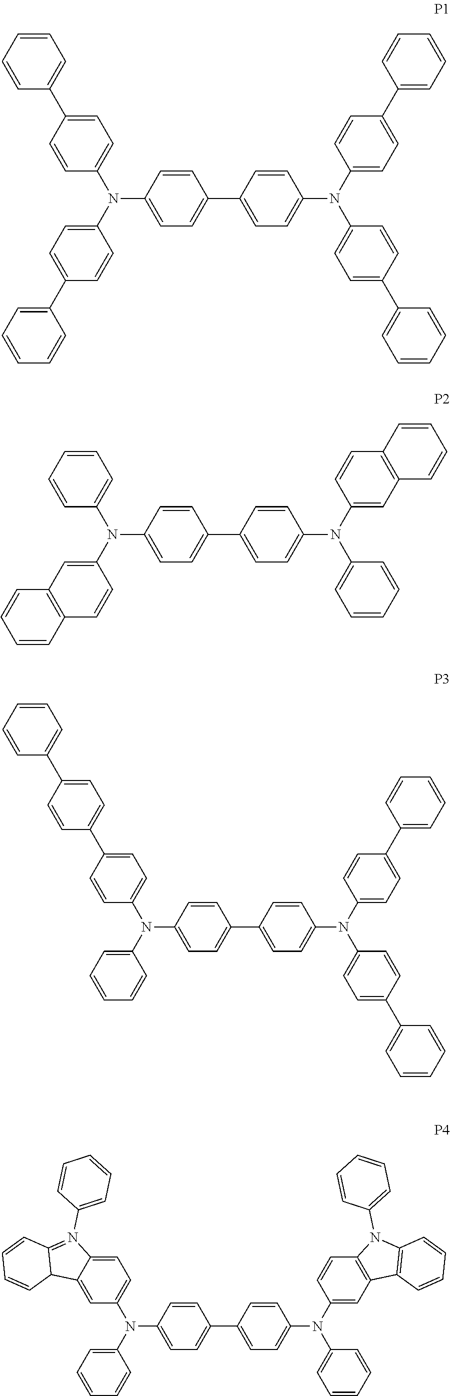

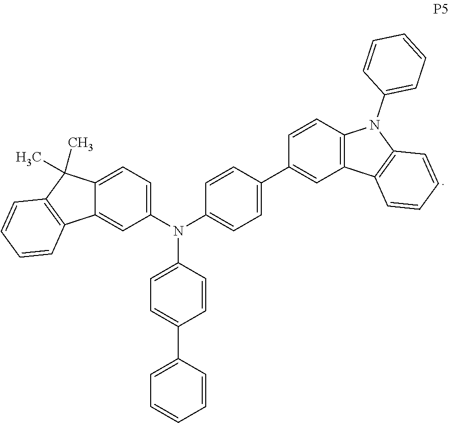 [SMILES: CC1(C)c2ccccc2-c2cc(N(c3ccc(-c4ccccc4)cc3)c3ccc(-c4ccc5c(c4)c4ccccc4n5-c4ccccc4)cc3)ccc21]

However, embodiments of the present disclosure are not limited thereto, and the capping layer CPL may include an amine compound. For example, the capping layer CPL may include either compound CPL1 or CPL2.

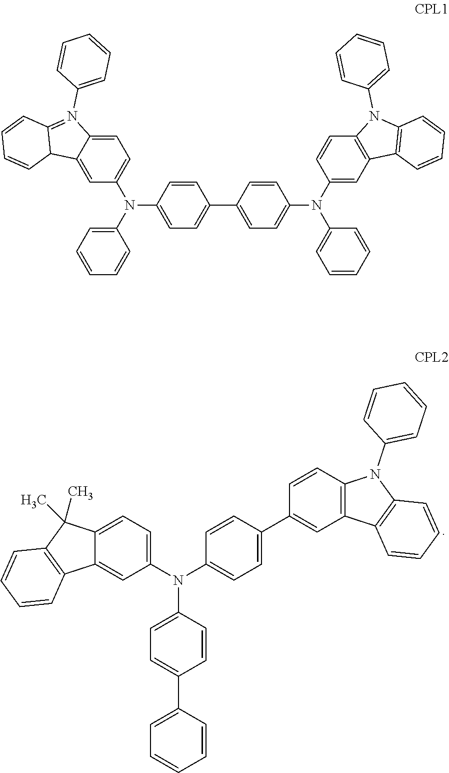

In some embodiments, the capping layer CPL may have a refractive index of 1.6 or more. For example, the capping layer CPL may have a refractive index of 1.6 or more for light in a wavelength range of 550 nm to 660 nm.

The aforementioned polycyclic compound of an embodiment may be included as a material for an organic electroluminescent element 10 in a functional layer other than the emission layer EML. The organic electroluminescent element 10 according to an embodiment of the present disclosure may include the aforementioned polycyclic compound in at least one functional layer disposed between the first electrode EL1 and the second electrode EL2, or in the capping layer CPL disposed on the second electrode EL2.

The polycyclic compound of an embodiment may include a first benzene ring and a second benzene ring, which are linked by a single bond, and carbazole moieties respectively substituted in an ortho position of the first benzene ring and in a meta position of the second benzene ring with respect to the single bond, and may thus exhibit high binding energy and/or high triplet energy. An additional carbazole group may be substituted on at least one of the carbazole moieties on each benzene ring of the biphenyl to improve hole transport ability. The polycyclic compound of an embodiment may be used in an emission layer of an organic electroluminescent element to improve luminous efficiency characteristics and service life.

Hereinafter, a polycyclic compound according to an embodiment of the present disclosure and an organic electroluminescent element of an embodiment including the polycyclic compound of an embodiment will be described in more detail with reference to embodiments and comparative embodiments. The following examples are only illustrations to assist the understanding of the present disclosure, and the scope of the present disclosure is not limited thereto.

### EXAMPLES

### 1. Synthesis of a Compound of an Embodiment.

First, a synthetic method of a compound according to an embodiment will be explained in more detail by illustrating the synthetic methods of Compound 1. The synthetic methods of the compounds explained below are only examples, and the synthetic method of the compound according to an embodiment of the present disclosure is not limited thereto.

### 1-1. Synthesis of Compound 1

Polycyclic Compound 1 according to an embodiment of the present disclosure may be synthesized by, for example, Reaction Scheme 1:

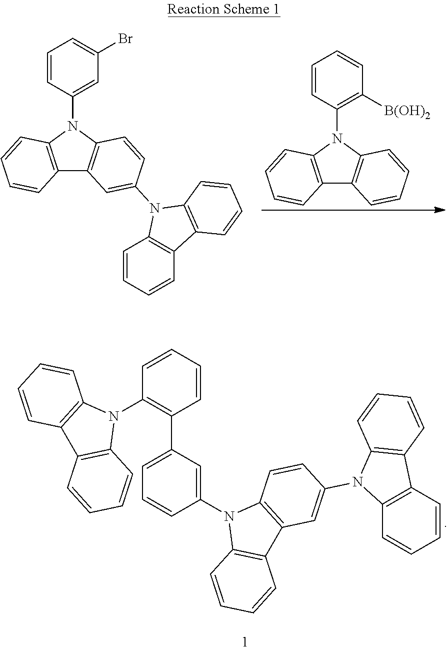

### Synthesis of Compound 1

9-(3-bromophenyl)-9H-3,9′-bicarbazole (2 g), 2-(9H-carbazol-9-yl)phenyl)boronic acid (1.18 g), 2 M potassium carbonate (K₂CO₃) solution (4.1 mL), and tetrakis(triphenylphosphine)palladium(0)) (Pd(PPh₃)₄) (0.24 g) were added to 20 mL of tetrahydrofuran (THF), and stirred for about 12 hours at about 90° C. After the reaction was completed, the reaction solution was extracted to obtain an organic layer, and the resultant organic layer was dried. The residue was separated and purified by column chromatography, followed by sublimation purification to obtain 2.5 g of Compound 1 (94% yield). Compound 1 was confirmed by LC-MS. (C₄₈H₃₁N₃: M+1 649.25)

### 1-2. Synthesis of Compound 3

Polycyclic Compound 3 according to an embodiment of the present disclosure may be synthesized by, for example, Reaction Scheme 2:

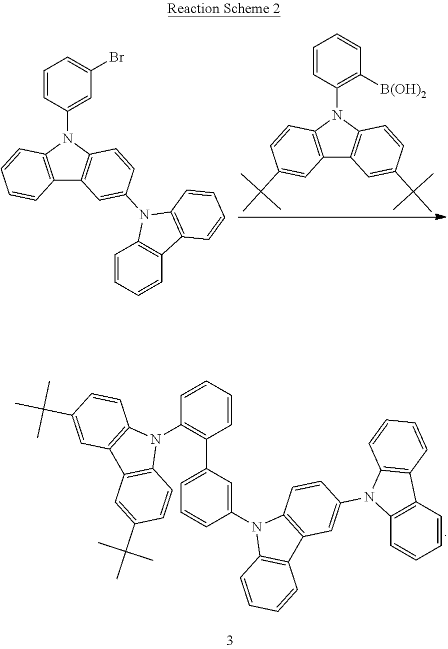

### Synthesis of Compound 3

9-(3-bromophenyl)-9H-3,9′-bicarbazole (2 g), (2-(3,6-di-tert-butyl-9H-carbazol-9-yl)phenyl)boronic acid (1.64 g), 2 M potassium carbonate (K₂CO₃) solution (4.1 mL), and tetrakis(triphenylphosphine)palladium(0)) (Pd(PPh₃)₄) (0.24 g) were added to 20 mL of tetrahydrofuran (THF), and stirred for about 12 hours at about 90° C. After the reaction was completed, the reaction solution was extracted to obtain an organic layer, and the resultant organic layer was dried. The residue was separated and purified by column chromatography, followed by sublimation purification to obtain 2.9 g of Compound 3 (93% yield). Compound 3 was confirmed by LC-MS. (C₅₆H₄₇N₃: M+1 762.01)

### 1-3. Synthesis of Compound 6

Polycyclic Compound 6 according to an embodiment of the present disclosure may be synthesized by, for example, Reaction Scheme 3:

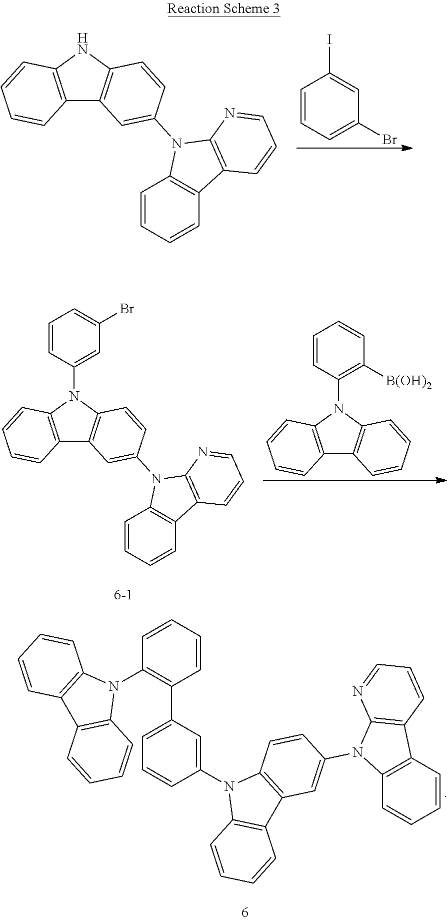

### Synthesis of Intermediate 6-1

9-(9H-carbazol-3-yl)-9H-pyrido[2,3-b]indole (3 g), 1-bromo-3-iodobenzene (3.05 g), potassium phosphate tribasic (K₃PO₄) (5.73 g), copper (I) iodide (CuI) (3.42 g), and ethylenediamine (0.3 mL) were added to 40 mL of toluene solvent, and the mixture was reacted at about 110° C. and then purified to obtain 4 g of Intermediate 6-1 (91% yield). Intermediate 6-1 was confirmed by LC-MS. (C₂₉H₁₈BrN₃: M+1 488.39)

### Synthesis of Compound 6

9-(9-(3-bromophenyl)-9H-carbazol-3-yl)-9H-pyrido[2,3-b]indole (2 g), (2-(9H-carbazol-9-yl)phenyl)boronic acid (1.18 g), 2 M potassium carbonate (K₂CO₃) solution (4.1 mL), and tetrakis(triphenylphosphine)palladium(0)) (Pd(PPh₃)₄) (0.24 g) were added to 20 mL of tetrahydrofuran (THF), and stirred for about 12 hours at about 90° C. After the reaction was completed, the reaction solution was extracted to obtain an organic layer, and the resultant organic layer was dried. The residue was separated and purified by column chromatography, followed by sublimation purification to give 2.2 g of Compound 6 (82% yield). Compound 6 was confirmed by LC-MS. (C₄₇H₃₀N₄: M+1 650.79)

### 1-4. Synthesis of Compound 35

Polycyclic Compound 35 according to an embodiment of the present disclosure may be synthesized by, for example, Reaction Scheme 4:

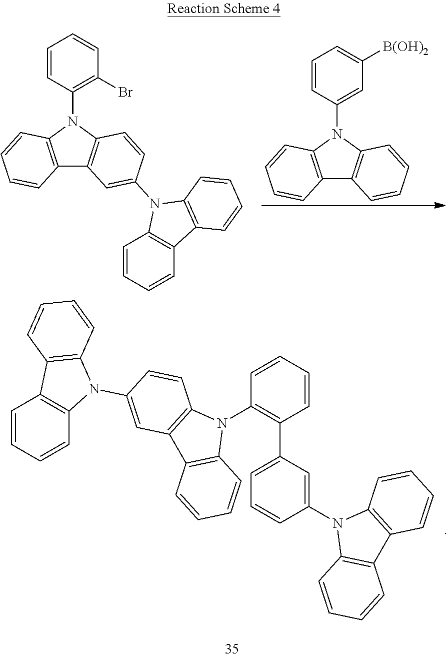

### Synthesis of Compound 35

9-(2-bromophenyl)-9H-3,9′-bicarbazole (2 g), (3-(9H-carbazol-9-yl)phenyl)boronic acid (1.18 g), 2 M potassium carbonate (K₂CO₃) solution (4.1 mL), and tetrakis(triphenylphosphine)palladium(0)) (Pd(PPh₃)₄) (0.24 g) were added to 20 mL of tetrahydrofuran (THF), and stirred for about 12 hours at about 90° C. After the reaction was completed, the reaction solution was extracted to obtain an organic layer, and the resultant organic layer was dried. The residue was separated and purified by column chromatography, followed by sublimation purification to obtain 2.4 g of Compound 35 (90% yield). Compound 35 was confirmed by LC-MS. (C₄₈H₃₁N₃: M+1 649.8)

### 1-5. Synthesis of Compound 37

Polycyclic Compound 37 according to an embodiment of the present disclosure may be synthesized by, for example, Reaction Scheme 5:

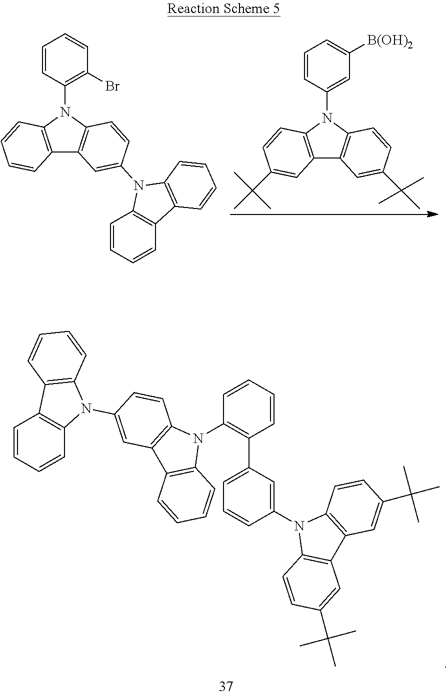

### Synthesis of Compound 37

9-(2-bromophenyl)-9H-3,9′-bicarbazole (2 g), (3-(3,6-di-tert-butyl-9H-carbazol-9-yl)phenyl)boronic acid (1.64 g), 2 M potassium carbonate (K₂CO₃) solution (4.1 mL), and tetrakis(triphenylphosphine)palladium(0)) (Pd(PPh₃)₄) (0.24 g) were added to 20 mL of tetrahydrofuran (THF), and stirred for about 12 hours at about 90° C. After the reaction was completed, the reaction solution was extracted to obtain an organic layer, and the resultant organic layer was dried. The residue was separated and purified by column chromatography, followed by sublimation purification to obtain 2.9 g of Compound 37 (93% yield). Compound 37 was confirmed by LC-MS. (C₅₆H₄₇N₃: M+1 762.01)

### 1-6. Synthesis of Compound 52

Polycyclic Compound 52 according to an embodiment of the present disclosure may be synthesized by, for example, Reaction Scheme 6:

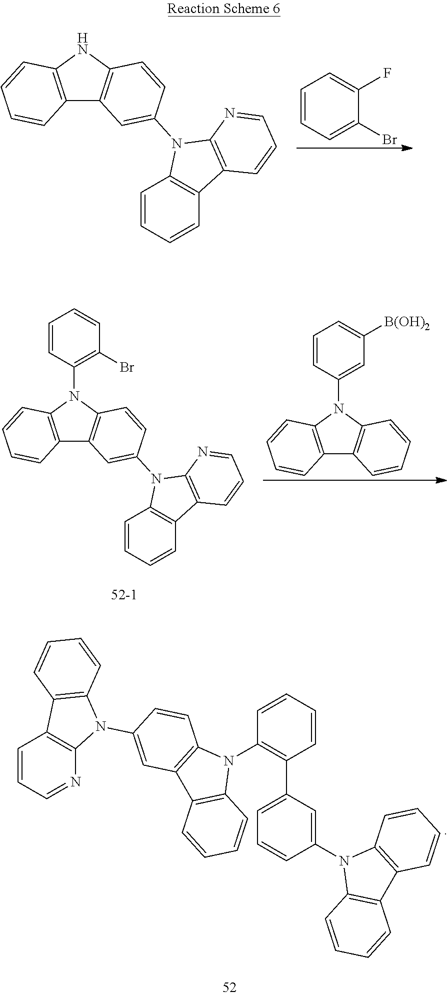

### Synthesis of Intermediate 52-1

9-(9H-carbazol-3-yl)-9H-pyrido[2,3-b]indole (3 g), 1-bromo-2-fluorobenzene (2.36 g), and potassium phosphate tribasic (K₃PO₄) (3.82 g) were added to 40 mL of dimethylformamide (DMF) solvent, and the mixture was reacted at about 160° C., and then purified to obtain 4 g of Intermediate 52-1 (91% yield). Intermediate 52-1 was confirmed by LC-MS. (C₂₉H₁₈BrN₃: M+1 488.39)

### Synthesis of Compound 52

9-(9-(2-bromophenyl)-9H-carbazol-3-yl)-9H-pyrido[2,3-b]indole (2 g), (3-(9H-carbazol-9-yl)phenyl)boronic acid (1.18 g), 2 M potassium carbonate (K₂CO₃) solution (4.1 mL), and tetrakis(triphenylphosphine)palladium(0)) (Pd(PPh₃)₄) (0.24 g) were added to 20 mL of tetrahydrofuran (THF), and stirred for about 12 hours at about 90° C. After the reaction was completed, the reaction solution was extracted to obtain an organic layer, and the resultant organic layer was dried. The residue was separated and purified by column chromatography, followed by sublimation purification to provide 2.4 g of Compound 52 (89% yield). Compound 52 was confirmed by LC-MS. (C₄₇H₃₀N₄: M+1 650.79)

### 1-7. Synthesis of Compound 69

Polycyclic Compound 69 according to an embodiment of the present disclosure may be synthesized by, for example, Reaction 7 Scheme:

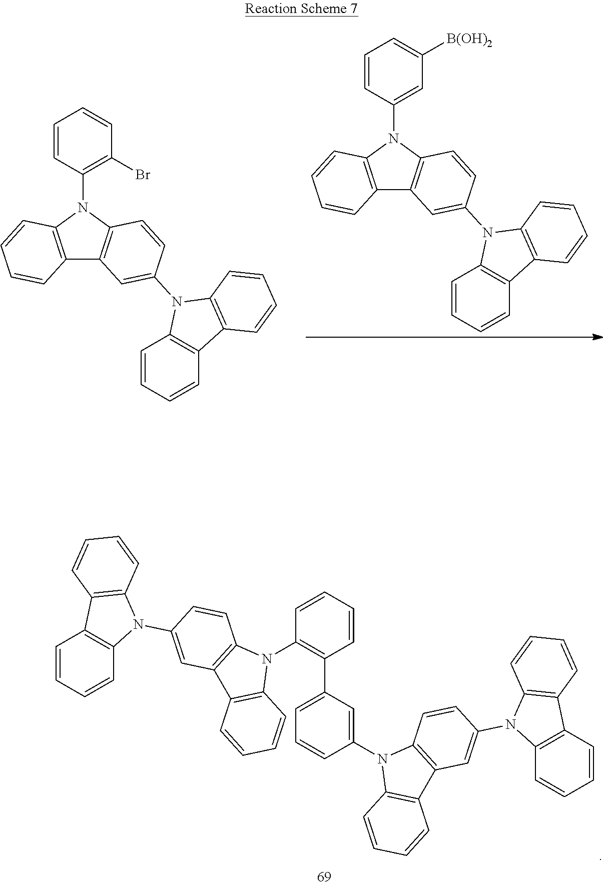 [SMILES: Brc1ccccc1-n1c2ccccc2c2cc(-n3c4ccccc4c4ccccc43)ccc21 ; OB(O)c1cccc(-n2c3ccccc3c3cc(-n4c5ccccc5c5ccccc54)ccc32)c1 ; c1cc(-c2ccccc2-n2c3ccccc3c3cc(-n4c5ccccc5c5ccccc54)ccc32)cc(-n2c3ccccc3c3cc(-n4c5ccccc5c5ccccc54)ccc32)c1]

### Synthesis of Compound 69

9-(2-bromophenyl)-9H-3,9′-bicarbazole (2 g), (3-(9H-[3,9′-bicarbazol]-9-yl)phenyl)boronic acid (1.86 g), 2 M potassium carbonate (K₂CO₃) solution (4.1 mL), and tetrakis(triphenylphosphine)palladium(0)) (Pd(PPh₃)₄) (0.24 g) were added to 20 mL of tetrahydrofuran (THF), and stirred for about 12 hours at about 90° C. After the reaction was completed, the reaction solution was extracted to obtain an organic layer, and the resultant organic layer was dried. The residue was separated and purified by column chromatography, followed by sublimation purification to obtain 3 g of Compound 69 (90% yield). Compound 69 was confirmed by LC-MS. (C₆₀H₃₈N₄: M+1 814.99)

### 2. Manufacture and Evaluation of an Organic Electroluminescent Element

An organic electroluminescent element of an embodiment, including a compound of an embodiment in an emission layer was evaluated by the method below. A method for manufacturing an organic electroluminescent element in order to evaluate the element is described below.

### Manufacture of an Organic Electroluminescent Element

For a first electrode (anode), an ITO glass substrate of about 15 Ω/cm² (about 1,200 Å) made by Corning Co. was cut to a size of 50 mm×50 mm×0.7 mm, cleansed by ultrasonic waves using isopropyl alcohol and pure water for about five minutes, and then irradiated with ultraviolet rays for about 30 minutes and exposed to ozone and cleansed. The glass substrate was installed on a vacuum deposition apparatus. On the upper portion of the substrate, NPD was first deposited in vacuum to form a 600 Å-thick hole injection layer, and then TCTA as a hole transporting compound was deposited in vacuum to form a 200 Å-thick hole transport layer. On the upper portion of the hole transport layer, CzSi, a hole transport layer compound, was deposited in vacuum to a thickness of about 100 Å. A polycyclic compound of an embodiment was co-deposited with Ir(pmp)₃ as a dopant material in a weight ratio of about 92:8 to form a 250 Å thick emission layer. Then, a layer with a thickness of about 200 Å was formed using TSPO1 as an electron transport layer compound, and TPBI as an electron injection layer compound was deposited to a thickness of about 300 Å. On the upper portion of the electron injection layer, LiF was deposited to form a 10 Å-thick electron injection layer, and Al was deposited in vacuum to form a 3,000 Å-thick LiF/Al second electrode (cathode) to manufacture an organic electroluminescent element.

### Evaluation of Characteristics of an Organic Electroluminescent Element

To evaluate the characteristics of the organic electroluminescent elements manufactured in Example 1 to Example 7 and Comparative Example 1 to Comparative Example 3, the driving voltage, luminous efficiency, and maximum external quantum efficiency (EQE) at the current density of about 10 mA/cm² of each of the organic electroluminescent elements were measured. The driving voltages of the organic electroluminescent elements were measured by using SourceMeter (Keithley Instrument, Inc., 2400 series), and the maximum external quantum efficiencies were measured by using an external quantum efficiency measurement apparatus, C9920-2-12 (manufactured by Hamamatsu Photonics, Inc). In the evaluation of the maximum external quantum efficiency, luminance/current density were measured using a luminance meter calibrated for wavelength sensitivity, and the maximum external quantum efficiency was calculated assuming an angular luminance distribution (Lambertian), which assumes an ideal diffuse reflecting surface. The evaluation results of characteristics of the organic electroluminescent elements are shown in Table 1:

TABLE 1

| | | | | External | |
|---|---|---|---|---|---|
| | Light- | Driving | Effi- | quantum | |
| | emitting | voltage | ciency | efficiency | Emission |
| | material | (V) | (Cd/A) | (%) | color |
| Example 1 | 1 | 4.2 | 25.8 | 24.5 | Blue |
| Example 2 | 3 | 4.9 | 23.7 | 22.0 | Blue |
| Example 3 | 6 | 4.8 | 21.4 | 21.5 | Blue |
| Example 4 | 35 | 4.3 | 25.4 | 23 | Blue |
| Example 5 | 37 | 4.8 | 24.2 | 21.8 | Blue |
| Example 6 | 52 | 4.8 | 21.6 | 21.3 | Blue |
| Example 7 | 69 | 4.4 | 24.3 | 22.2 | Blue |
| Comparative | Compound A | 5.0 | 18.9 | 16.4 | Blue to |
| Example 1 | | | | | green |
| Comparative | Compound B | 5.2 | 18.6 | 17.1 | Blue |
| Example 2 | | | | | |
| Comparative | Compound C | 5.0 | 15.2 | 16.8 | Blue |
| Example 3 | | | | | |

### Comparative Example 1 to Comparative Example 3

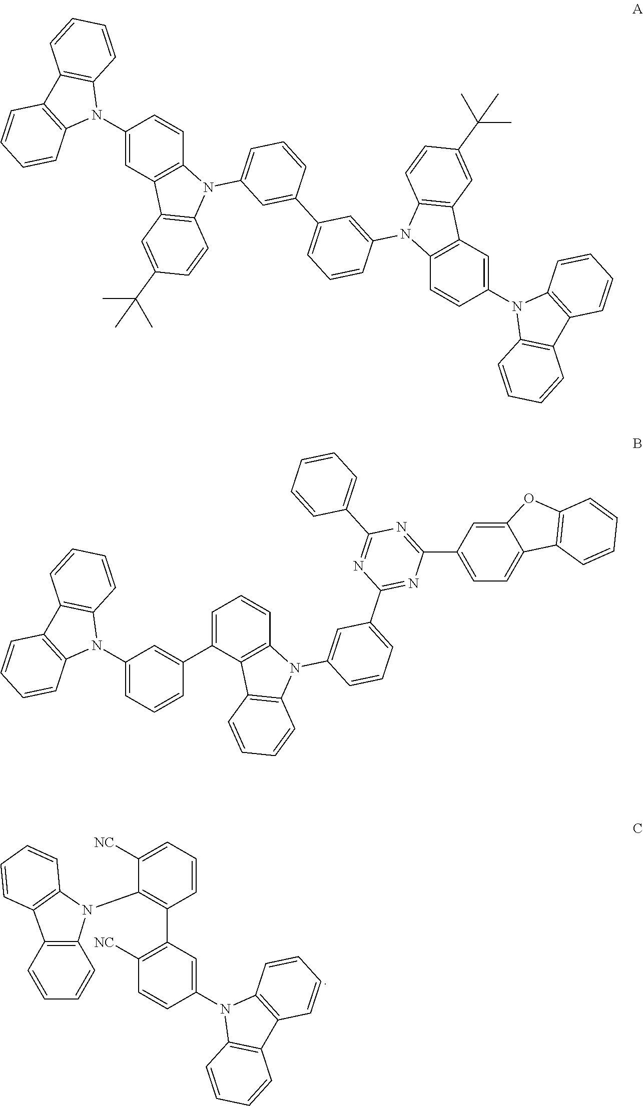 [SMILES: CC(C)(C)c1ccc2c(c1)c1cc(-n3c4ccccc4c4ccccc43)ccc1n2-c1cccc(-c2cccc(-n3c4ccc(-n5c6ccccc6c6ccccc65)cc4c4cc(C(C)(C)C)ccc43)c2)c1 ; c1ccc(-c2nc(-c3cccc(-n4c5ccccc5c5c(-c6cccc(-n7c8ccccc8c8ccccc87)c6)cccc54)c3)nc(-c3ccc4c(c3)oc3ccccc34)n2)cc1 ; N#Cc1ccc(-n2c3ccccc3c3ccccc32)cc1-c1cccc(C#N)c1-n1c2ccccc2c2ccccc21]

Referring to the experiment results of Table 1, when the polycyclic compound according to an embodiment was used as a host material for the emission layer of the organic electroluminescent element, low driving voltage, high efficiency, and long service life were achieved compared to the Comparative Examples. In particular, it may be seen that Examples 1 to 7 each exhibit high efficiency and long service life characteristics compared to Comparative Example 1 to 3.

It is believed that the polycyclic compounds in Examples exhibit desired or excellent luminous efficiency and long service life characteristics, compared with Comparative Example Compounds, by having a carbazole group substituted in the ortho-position of one phenyl group and a carbazole group substituted in the meta-position of the other phenyl group in biphenyl.

Comparing Example 1 to Example 7 with the Comparative Examples, Comparative Example 1 differs from Examples 1 to 7 in that the carbazole groups are substituted at the meta-position of both phenyl groups of biphenyl; Comparative Example 2 differs from Examples 1 to 7 in that the carbazole groups are not substituted at a biphenyl group, but at independent phenyl groups, and Comparative Example 3 differs from Examples 1 to 7 in that other substituents (e.g., multiple cyano groups) in addition to carbazole groups are further substituted in biphenyl. Thus, it may be seen that the Comparative Examples exhibit high driving voltage, low luminous efficiency and short service life characteristics compared to the Examples.

Thus, it may be confirmed that the Examples exhibit desired or excellent luminous efficiency and long service life characteristics, compared with the Comparative Example Compounds, due to each having a carbazole group substituted in the ortho-position of one phenyl group and a carbazole group substituted in the meta-position of the other phenyl group in biphenyl.

The polycyclic compound according to an embodiment may be used as a material for an emission layer of an organic electroluminescent element to achieve high efficiency of the organic electroluminescent element.

The organic electroluminescent element according to an embodiment may include the polycyclic compound of an embodiment to thereby achieve high efficiency.

The organic electroluminescent element of an embodiment may include the polycyclic compound of an embodiment, thereby exhibiting high efficiency characteristics in a blue wavelength region.

As used herein, the terms “substantially,” “about,” and similar terms are used as terms of approximation and not as terms of degree, and are intended to account for the inherent deviations in measured or calculated values that would be recognized by those of ordinary skill in the art.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

The polycyclic compound of an embodiment may improve the luminous efficiency of an organic electroluminescent element.

Although the example embodiments of the present disclosure have been described, it is understood that the present disclosure should not be limited to these example embodiments, but that various changes and modifications can be made by one ordinary skilled in the art within the spirit and scope of the present disclosure as defined in the following claims and equivalents thereof.

What is claimed is:

1. An organic electroluminescent element comprising:
a first electrode;
a second electrode facing the first electrode; and
a plurality of functional layers between the first electrode and the second electrode;
wherein at least one of the functional layers comprises a polycyclic compound, the polycyclic compound comprising:
a first benzene ring and a second benzene ring, which are linked by a single bond;
a first carbazole group substituted in an ortho position of the first benzene ring with respect to the single bond;
a second carbazole group substituted in a meta position of the second benzene ring with respect to the single bond; and
a third carbazole group substituted on at least one of the first carbazole group or the second carbazole group.

2. The organic electroluminescent element of claim 1, wherein the first carbazole group is in an opposite position to the second carbazole group with respect to the single bond.

3. The organic electroluminescent element of claim 1, wherein the functional layers comprise a hole transport region, an emission layer, and an electron transport region, and
wherein the emission layer comprises the polycyclic compound.

4. The organic electroluminescent element of claim 3, wherein the emission layer is to emit blue light.

5. The organic electroluminescent element of claim 3, wherein the emission layer comprises a host and a dopant, and the host comprises the polycyclic compound.

6. The organic electroluminescent element of claim 1, wherein the first carbazole group to the third carbazole group are each independently an unsubstituted carbazole group, or a carbazole group that is substituted with at least one selected from a deuterium atom, a halogen atom, a substituted or unsubstituted silyl group, a cyano group, a substituted or unsubstituted alkyl group having 1 to 10 carbon atoms, a substituted or unsubstituted ring-forming aryl group having 6 to 30 carbon atoms, and a substituted or unsubstituted ring-forming heteroaryl group having 2 to 30 carbon atoms.

7. The organic electroluminescent element of claim 1, wherein the first carbazole group to the third carbazole group are each independently represented by one selected from Formula C1 to Formula C10:

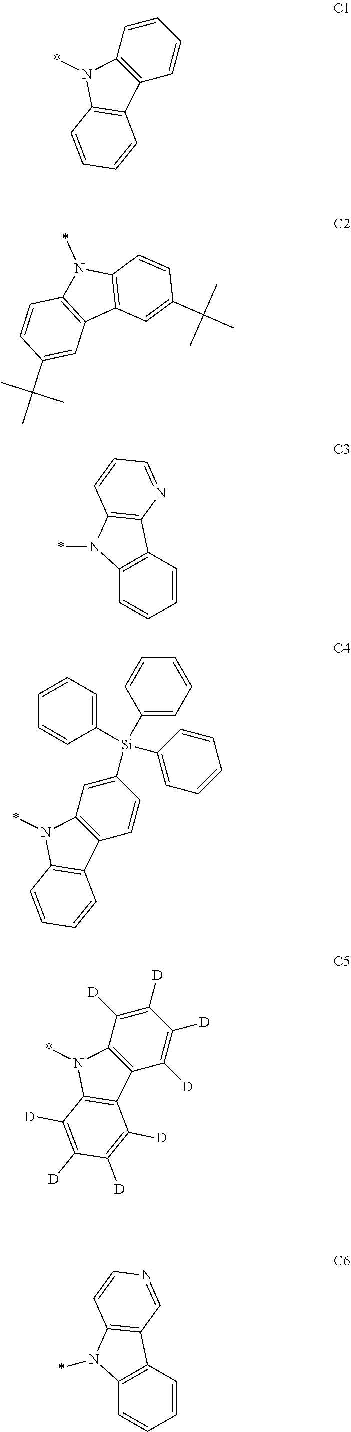

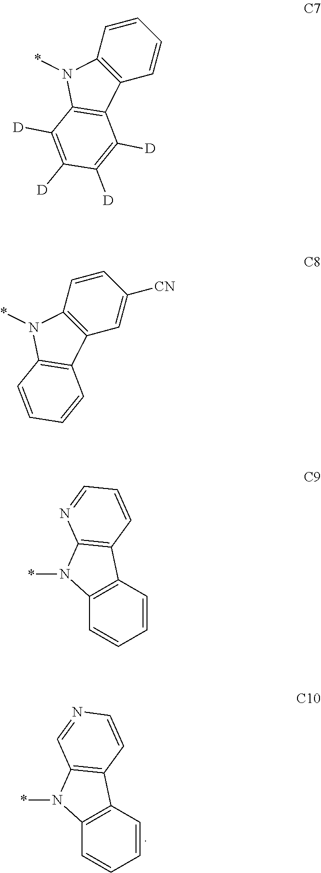 [SMILES: *n1c2ccccc2c2c([2H])c([2H])c([2H])c([2H])c21 ; *n1c2ccccc2c2cc(C#N)ccc21 ; *n1c2ccccc2c2cccnc21 ; *n1c2ccccc2c2ccncc21]

8. The organic electroluminescent element of claim 1, wherein the polycyclic compound is represented by one selected from the polycyclic compounds in Compound Group 1:

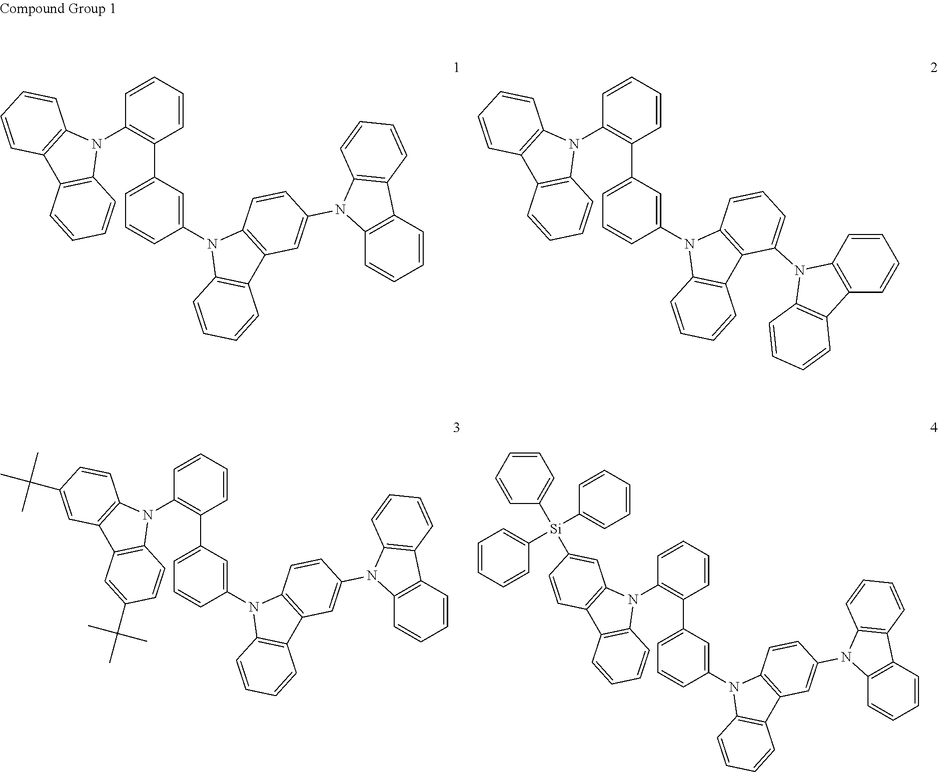

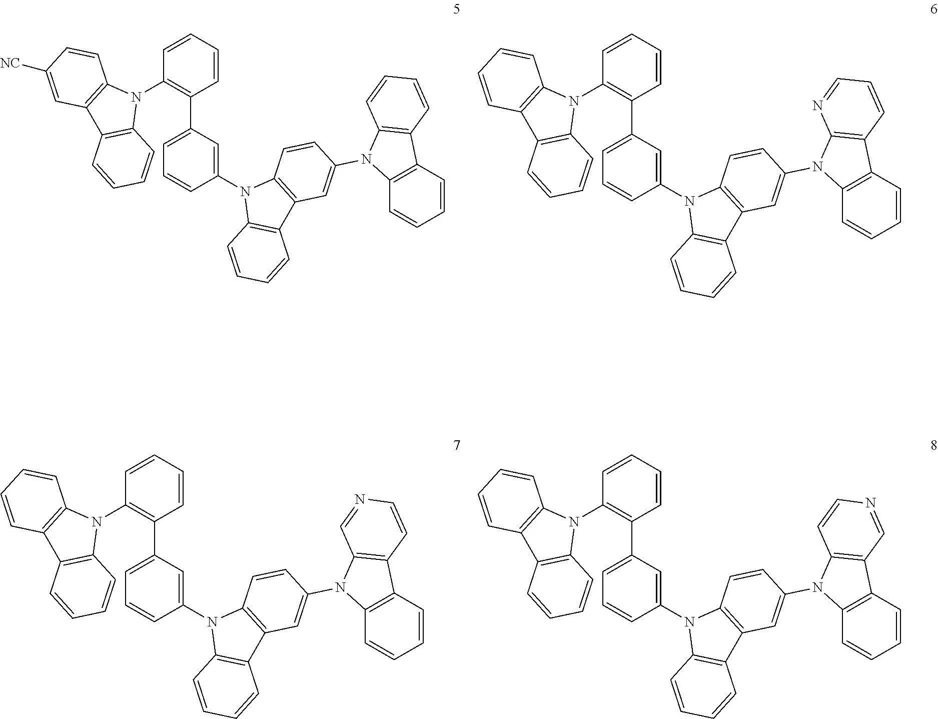 [SMILES: N#Cc1ccc2c(c1)c1ccccc1n2-c1ccccc1-c1cccc(-n2c3ccccc3c3cc(-n4c5ccccc5c5ccccc54)ccc32)c1 ; c1cc(-c2ccccc2-n2c3ccccc3c3ccccc32)cc(-n2c3ccccc3c3cc(-n4c5ccccc5c5cccnc54)ccc32)c1 ; c1cc(-c2ccccc2-n2c3ccccc3c3ccccc32)cc(-n2c3ccccc3c3cc(-n4c5ccccc5c5ccncc54)ccc32)c1 ; c1cc(-c2ccccc2-n2c3ccccc3c3ccccc32)cc(-n2c3ccccc3c3cc(-n4c5ccccc5c5cnccc54)ccc32)c1]

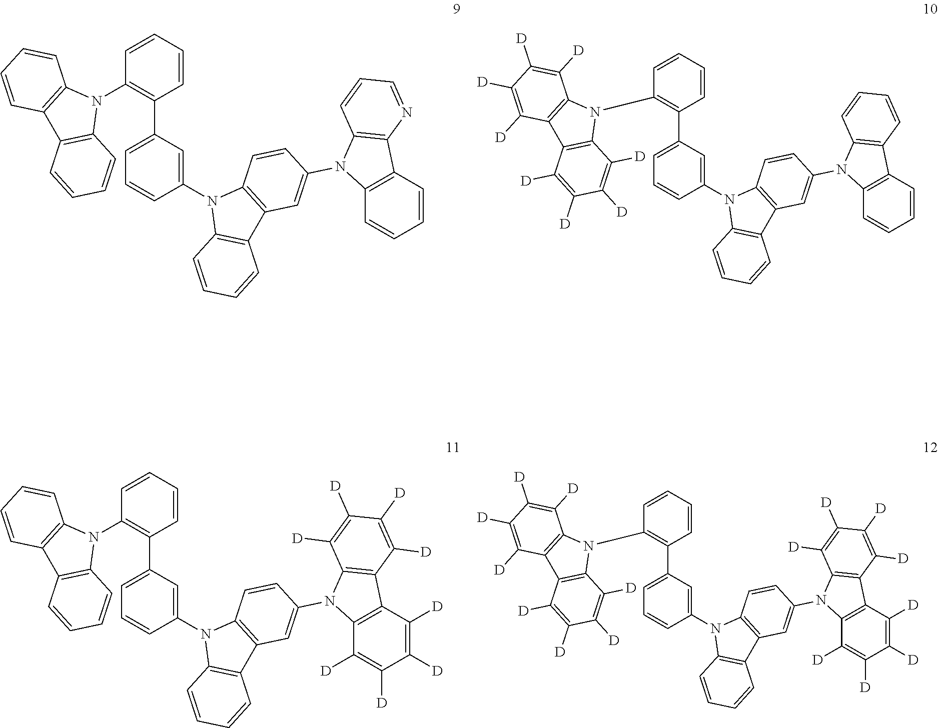

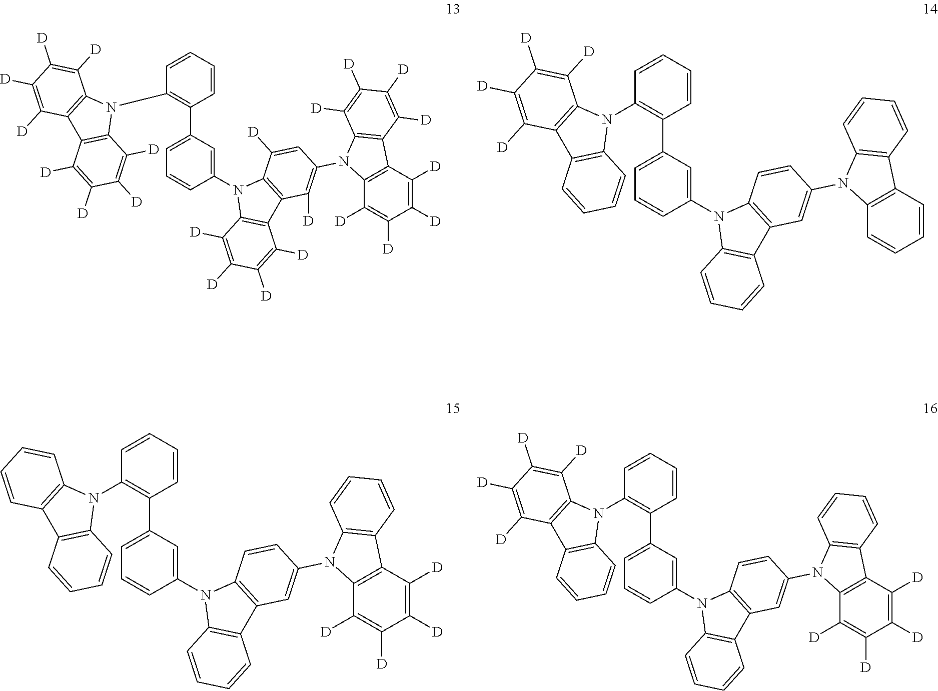

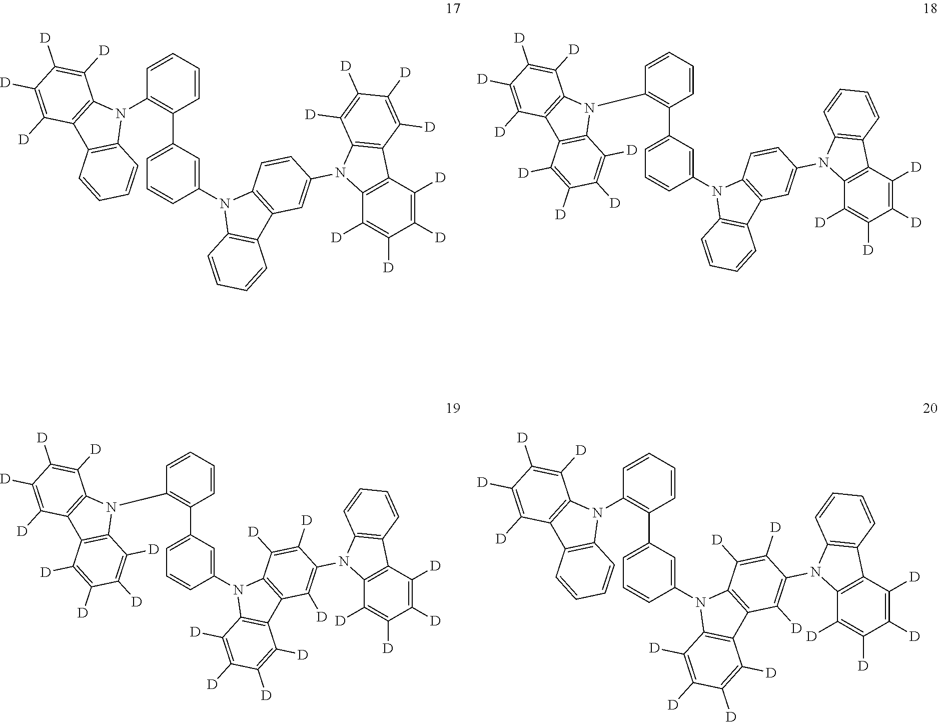

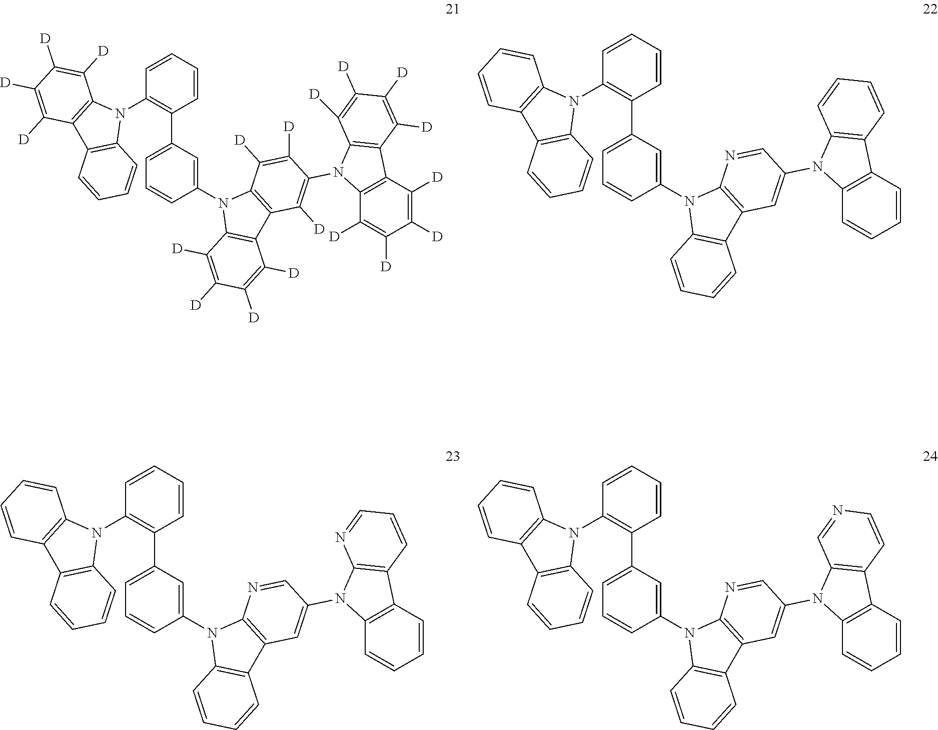 [SMILES: [2H]c1c([2H])c([2H])c2c(c1[2H])c1ccccc1n2-c1ccccc1-c1cccc(-n2c3c([2H])c([2H])c([2H])c([2H])c3c3c([2H])c(-n4c5c([2H])c([2H])c([2H])c([2H])c5c5c([2H])c([2H])c([2H])c([2H])c54)c([2H])c([2H])c32)c1 ; c1cc(-c2ccccc2-n2c3ccccc3c3ccccc32)cc(-n2c3ccccc3c3cc(-n4c5ccccc5c5ccccc54)cnc32)c1 ; c1cc(-c2ccccc2-n2c3ccccc3c3ccccc32)cc(-n2c3ccccc3c3cc(-n4c5ccccc5c5cccnc54)cnc32)c1 ; c1cc(-c2ccccc2-n2c3ccccc3c3ccccc32)cc(-n2c3ccccc3c3cc(-n4c5ccccc5c5ccncc54)cnc32)c1]

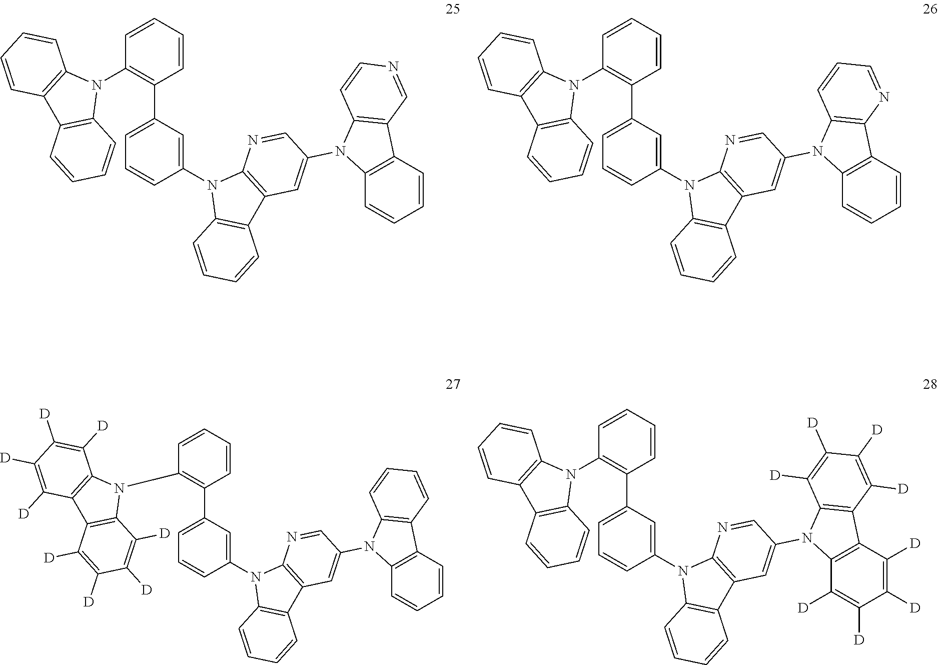 [SMILES: c1cc(-c2ccccc2-n2c3ccccc3c3ccccc32)cc(-n2c3ccccc3c3cc(-n4c5ccccc5c5cnccc54)cnc32)c1 ; c1cc(-c2ccccc2-n2c3ccccc3c3ccccc32)cc(-n2c3ccccc3c3cc(-n4c5ccccc5c5ncccc54)cnc32)c1 ; [2H]c1c([2H])c([2H])c2c(c1[2H])c1c([2H])c([2H])c([2H])c([2H])c1n2-c1ccccc1-c1cccc(-n2c3ccccc3c3cc(-n4c5ccccc5c5ccccc54)cnc32)c1 ; [2H]c1c([2H])c([2H])c2c(c1[2H])c1c([2H])c([2H])c([2H])c([2H])c1n2-c1cnc2c(c1)c1ccccc1n2-c1cccc(-c2ccccc2-n2c3ccccc3c3ccccc32)c1]

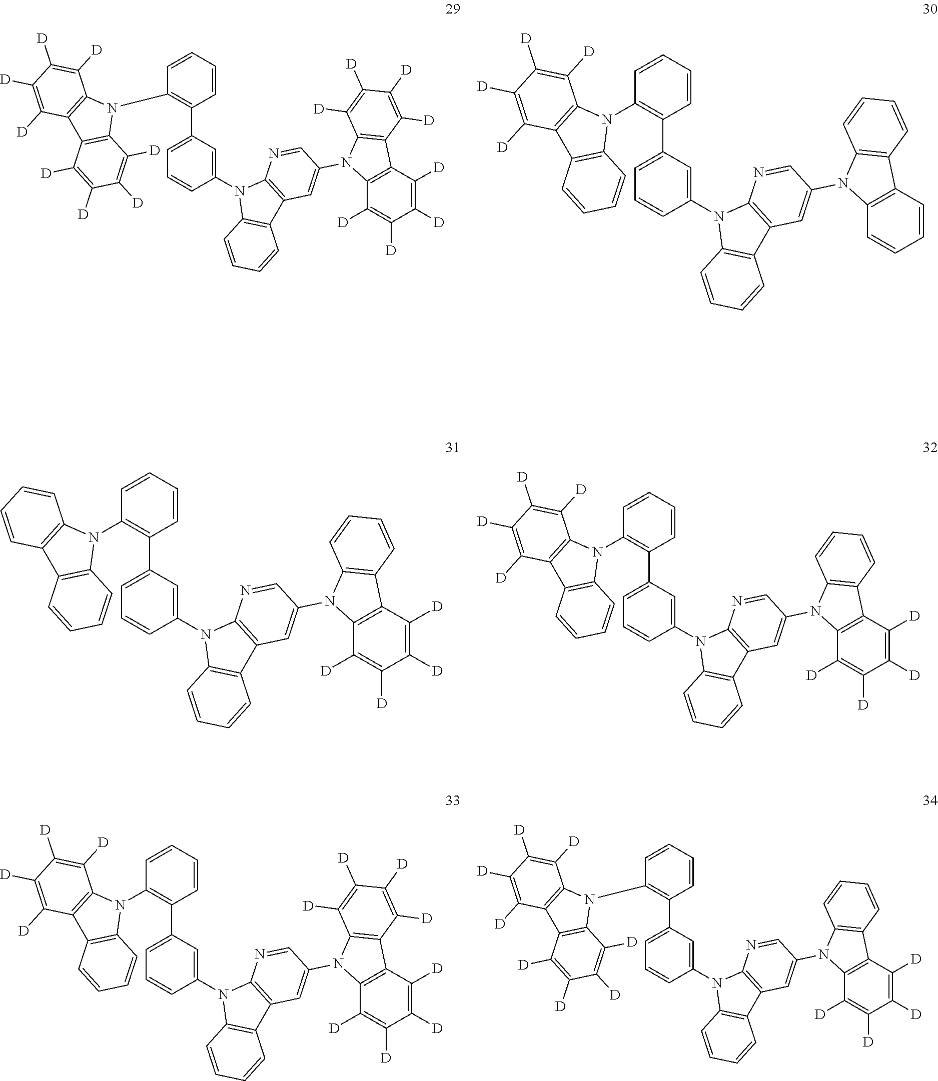

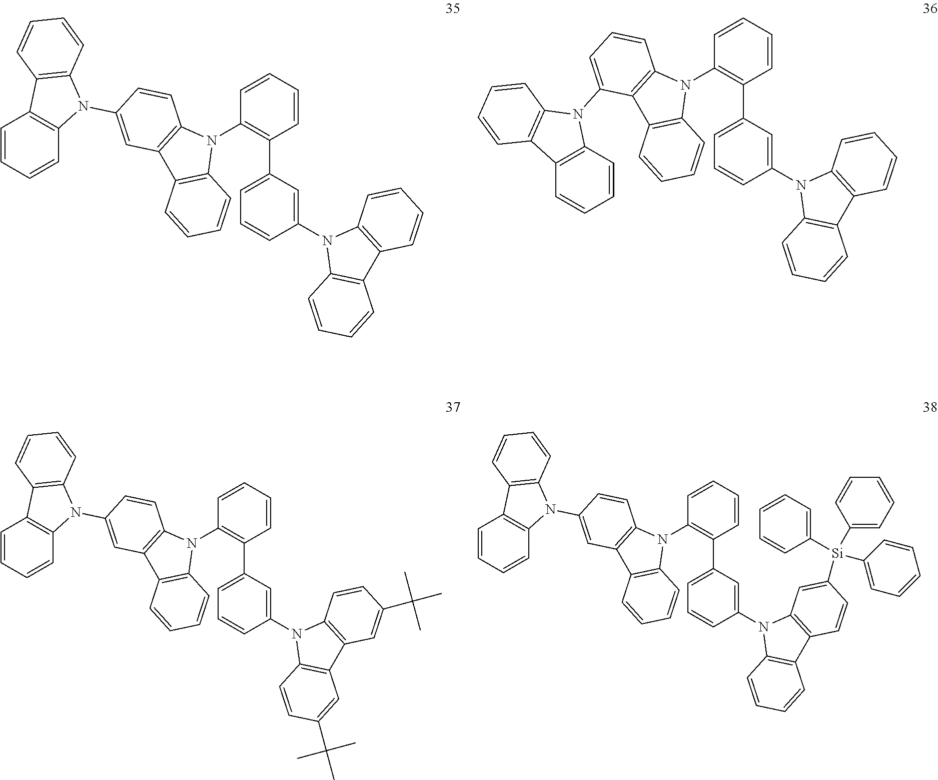 [SMILES: c1cc(-c2ccccc2-n2c3ccccc3c3cc(-n4c5ccccc5c5ccccc54)ccc32)cc(-n2c3ccccc3c3ccccc32)c1 ; c1cc(-c2ccccc2-n2c3ccccc3c3c(-n4c5ccccc5c5ccccc54)cccc32)cc(-n2c3ccccc3c3ccccc32)c1 ; CC(C)(C)c1ccc2c(c1)c1cc(C(C)(C)C)ccc1n2-c1cccc(-c2ccccc2-n2c3ccccc3c3cc(-n4c5ccccc5c5ccccc54)ccc32)c1 ; c1ccc([Si](c2ccccc2)(c2ccccc2)c2ccc3c4ccccc4n(-c4cccc(-c5ccccc5-n5c6ccccc6c6cc(-n7c8ccccc8c8ccccc87)ccc65)c4)c3c2)cc1]

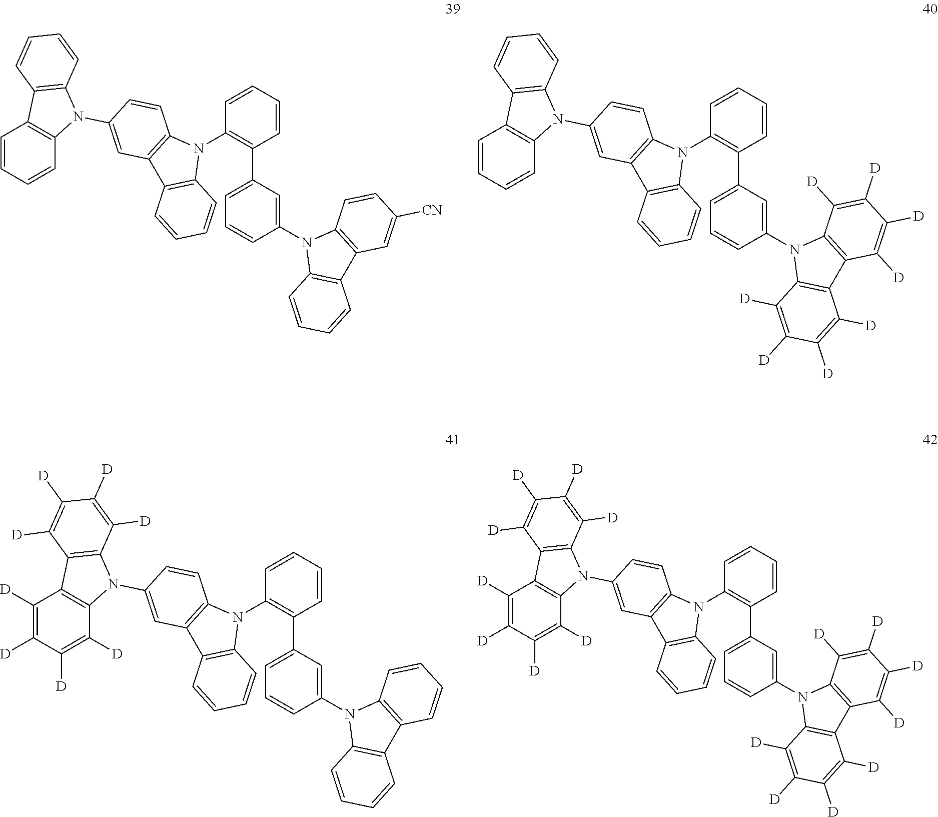

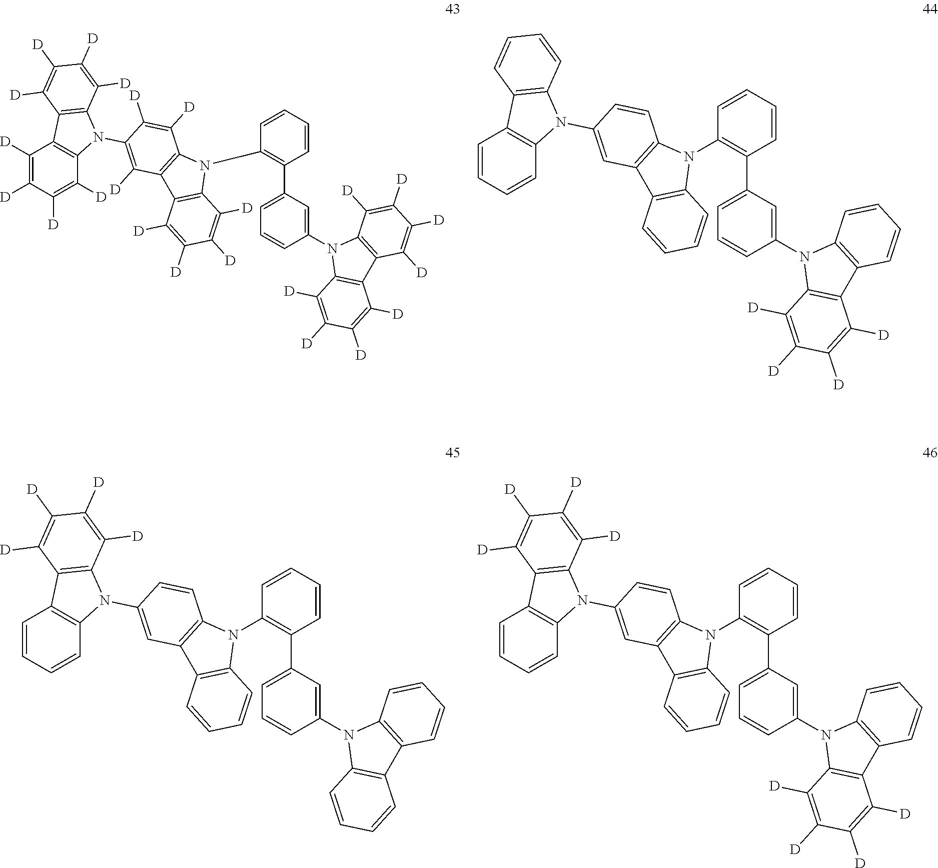

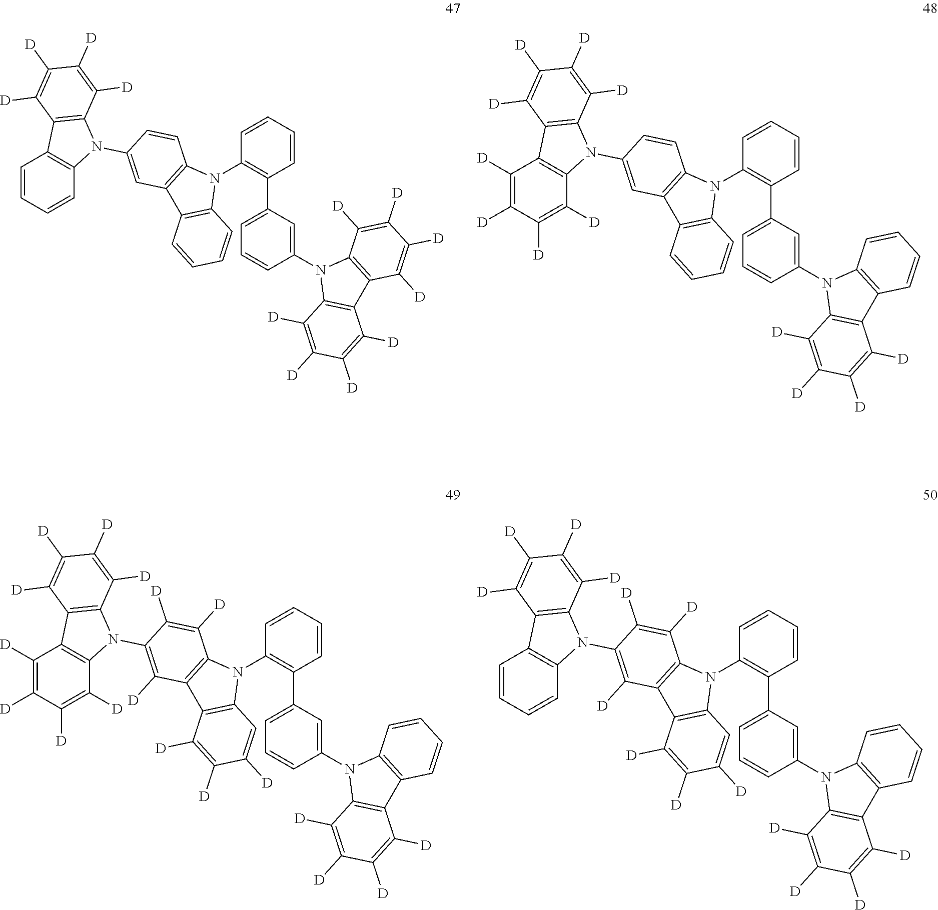

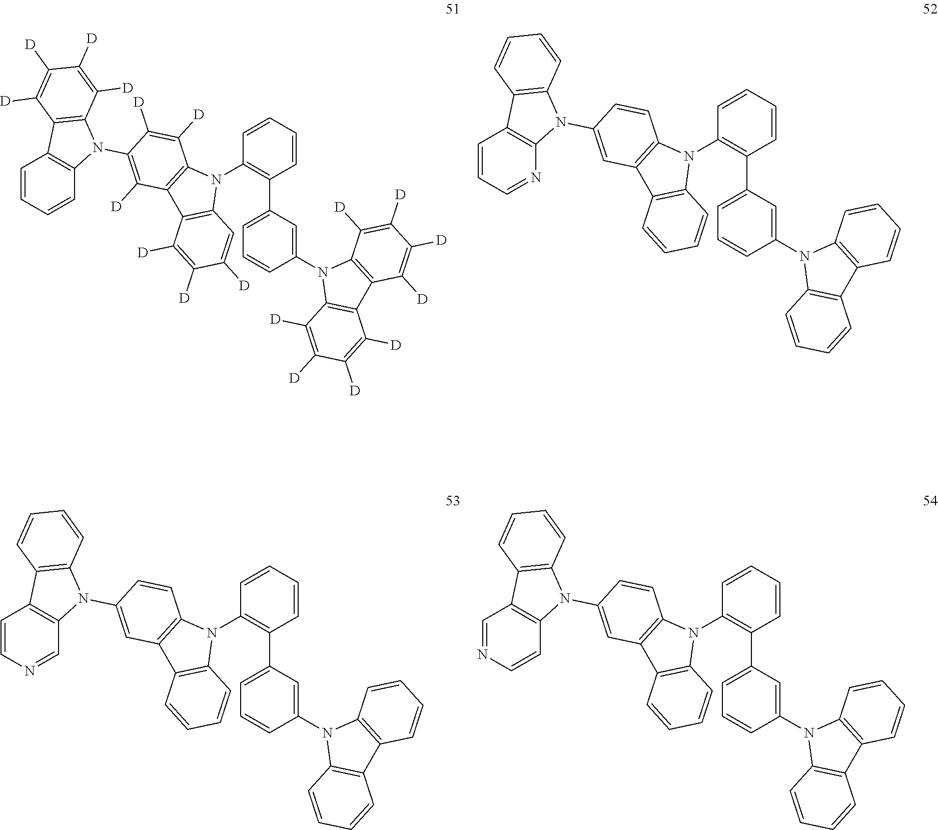 [SMILES: [2H]c1cc2c(c([2H])c1[2H])c1c([2H])c(-n3c4ccccc4c4c([2H])c([2H])c([2H])c([2H])c43)c([2H])c([2H])c1n2-c1ccccc1-c1cccc(-n2c3c([2H])c([2H])c([2H])c([2H])c3c3c([2H])c([2H])c([2H])c([2H])c32)c1 ; c1cc(-c2ccccc2-n2c3ccccc3c3cc(-n4c5ccccc5c5cccnc54)ccc32)cc(-n2c3ccccc3c3ccccc32)c1 ; c1cc(-c2ccccc2-n2c3ccccc3c3cc(-n4c5ccccc5c5ccncc54)ccc32)cc(-n2c3ccccc3c3ccccc32)c1 ; c1cc(-c2ccccc2-n2c3ccccc3c3cc(-n4c5ccccc5c5cnccc54)ccc32)cc(-n2c3ccccc3c3ccccc32)c1]

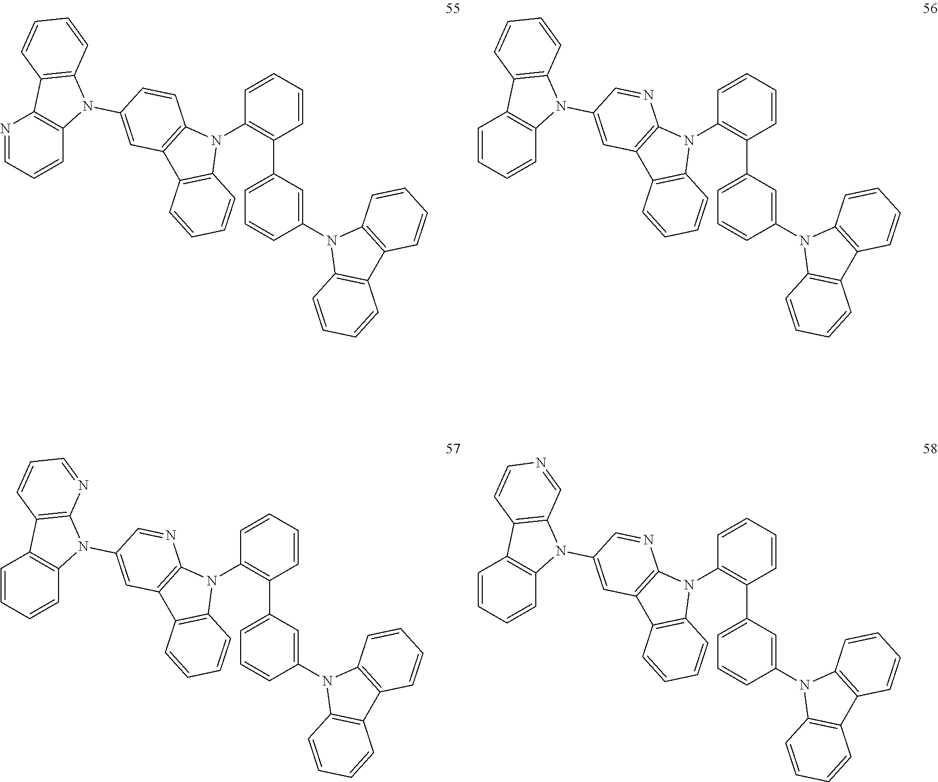 [SMILES: c1cc(-c2ccccc2-n2c3ccccc3c3cc(-n4c5ccccc5c5ncccc54)ccc32)cc(-n2c3ccccc3c3ccccc32)c1 ; c1cc(-c2ccccc2-n2c3ccccc3c3cc(-n4c5ccccc5c5ccccc54)cnc32)cc(-n2c3ccccc3c3ccccc32)c1 ; c1cc(-c2ccccc2-n2c3ccccc3c3cc(-n4c5ccccc5c5cccnc54)cnc32)cc(-n2c3ccccc3c3ccccc32)c1 ; c1cc(-c2ccccc2-n2c3ccccc3c3cc(-n4c5ccccc5c5ccncc54)cnc32)cc(-n2c3ccccc3c3ccccc32)c1]

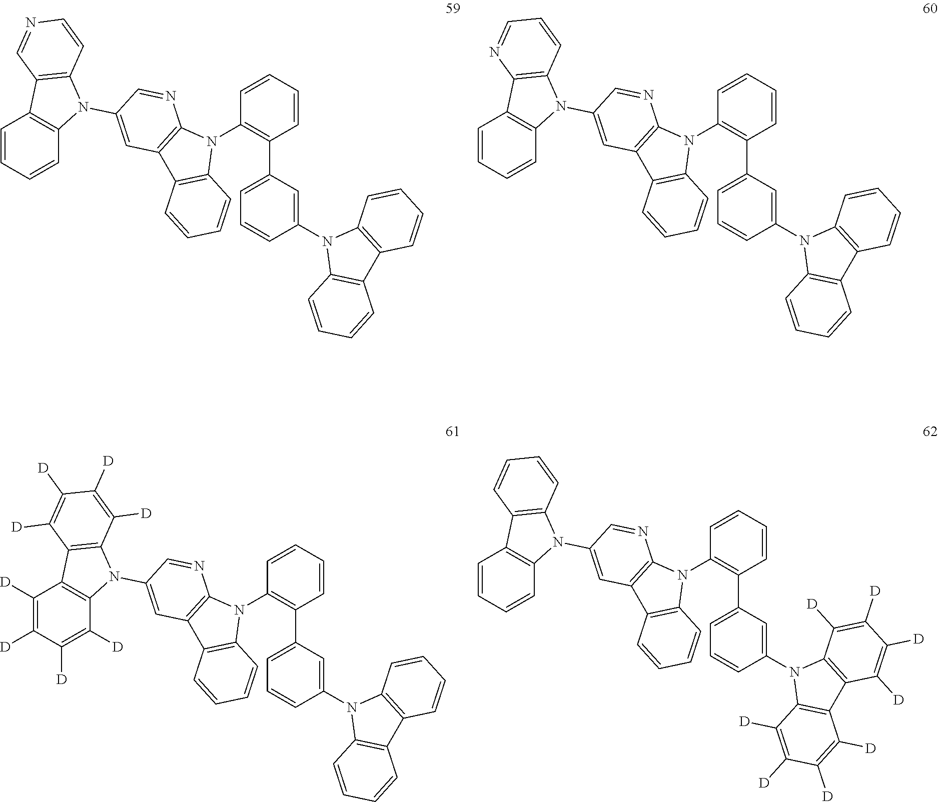 [SMILES: c1cc(-c2ccccc2-n2c3ccccc3c3cc(-n4c5ccccc5c5cnccc54)cnc32)cc(-n2c3ccccc3c3ccccc32)c1 ; c1cc(-c2ccccc2-n2c3ccccc3c3cc(-n4c5ccccc5c5ncccc54)cnc32)cc(-n2c3ccccc3c3ccccc32)c1 ; [2H]c1c([2H])c([2H])c2c(c1[2H])c1c([2H])c([2H])c([2H])c([2H])c1n2-c1cnc2c(c1)c1ccccc1n2-c1ccccc1-c1cccc(-n2c3ccccc3c3ccccc32)c1 ; [2H]c1c([2H])c([2H])c2c(c1[2H])c1c([2H])c([2H])c([2H])c([2H])c1n2-c1cccc(-c2ccccc2-n2c3ccccc3c3cc(-n4c5ccccc5c5ccccc54)cnc32)c1]

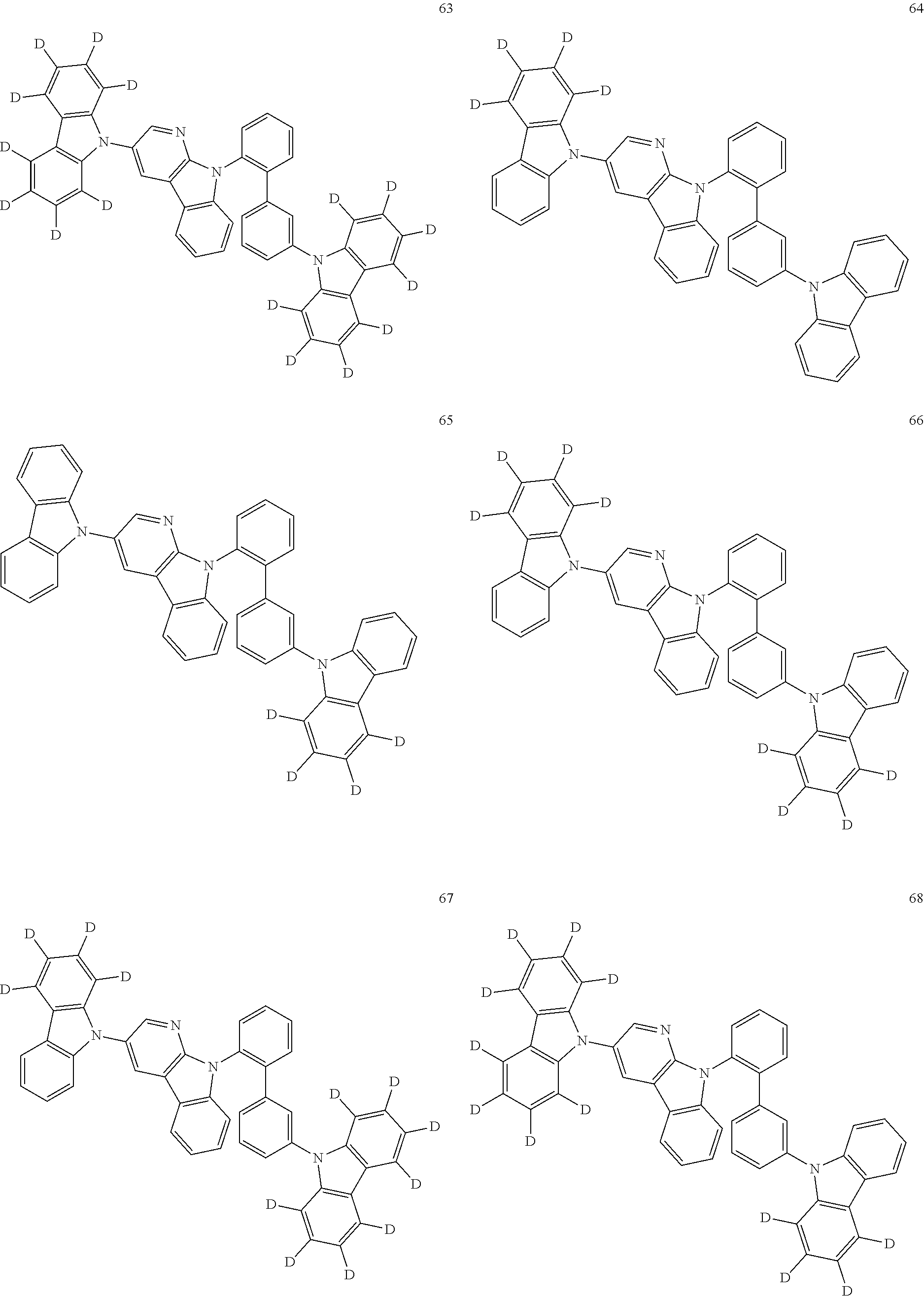

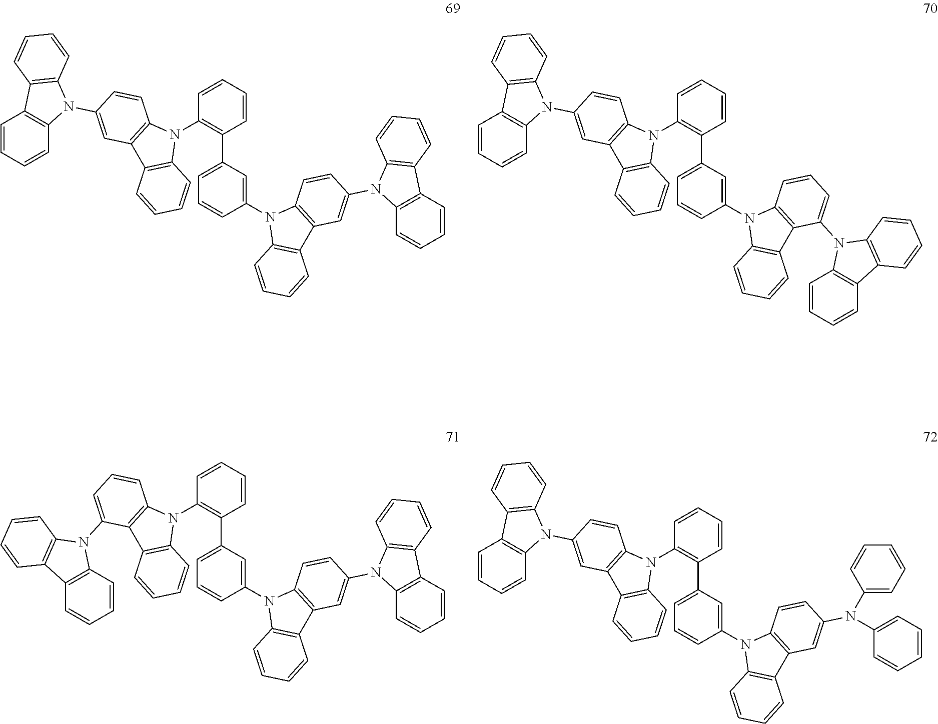

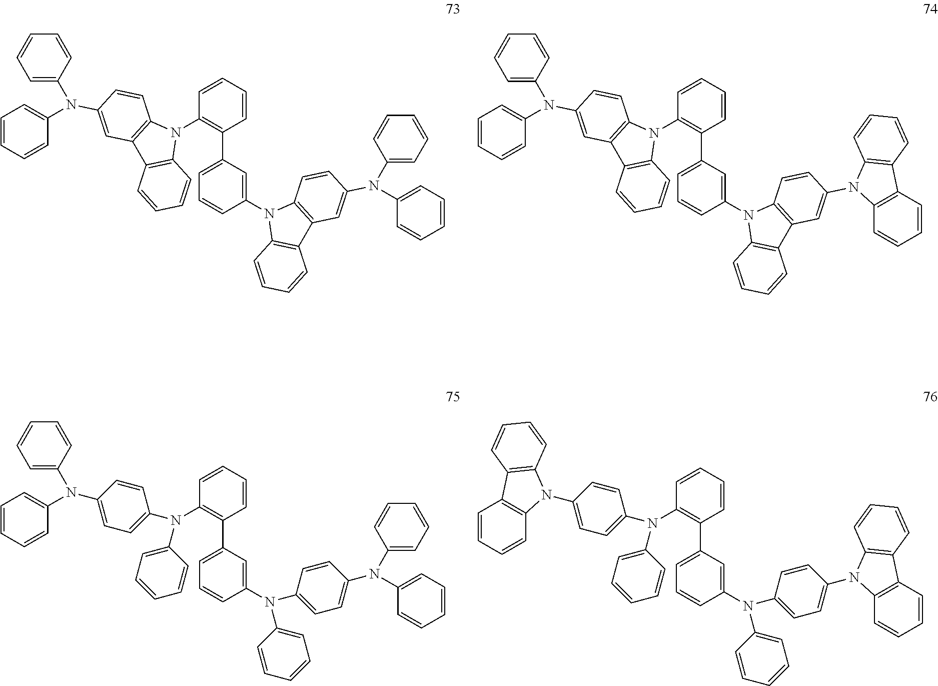 [SMILES: c1ccc(N(c2ccccc2)c2ccc3c(c2)c2ccccc2n3-c2cccc(-c3ccccc3-n3c4ccccc4c4cc(N(c5ccccc5)c5ccccc5)ccc43)c2)cc1 ; c1ccc(N(c2ccccc2)c2ccc3c(c2)c2ccccc2n3-c2ccccc2-c2cccc(-n3c4ccccc4c4cc(-n5c6ccccc6c6ccccc65)ccc43)c2)cc1 ; c1ccc(N(c2ccccc2)c2ccc(N(c3ccccc3)c3cccc(-c4ccccc4N(c4ccccc4)c4ccc(N(c5ccccc5)c5ccccc5)cc4)c3)cc2)cc1 ; c1ccc(N(c2ccc(-n3c4ccccc4c4ccccc43)cc2)c2cccc(-c3ccccc3N(c3ccccc3)c3ccc(-n4c5ccccc5c5ccccc54)cc3)c2)cc1]

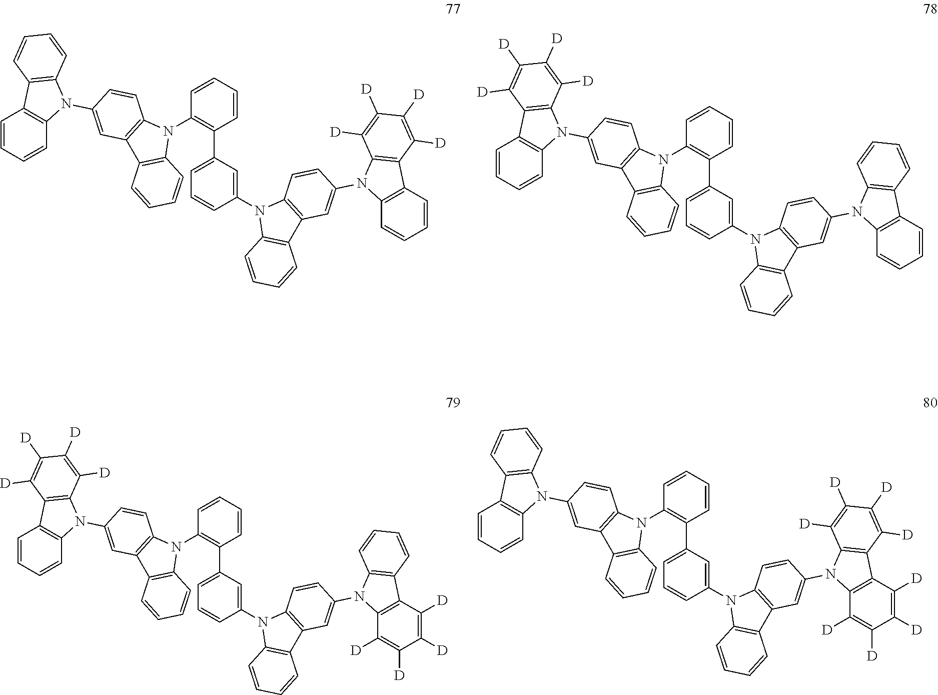

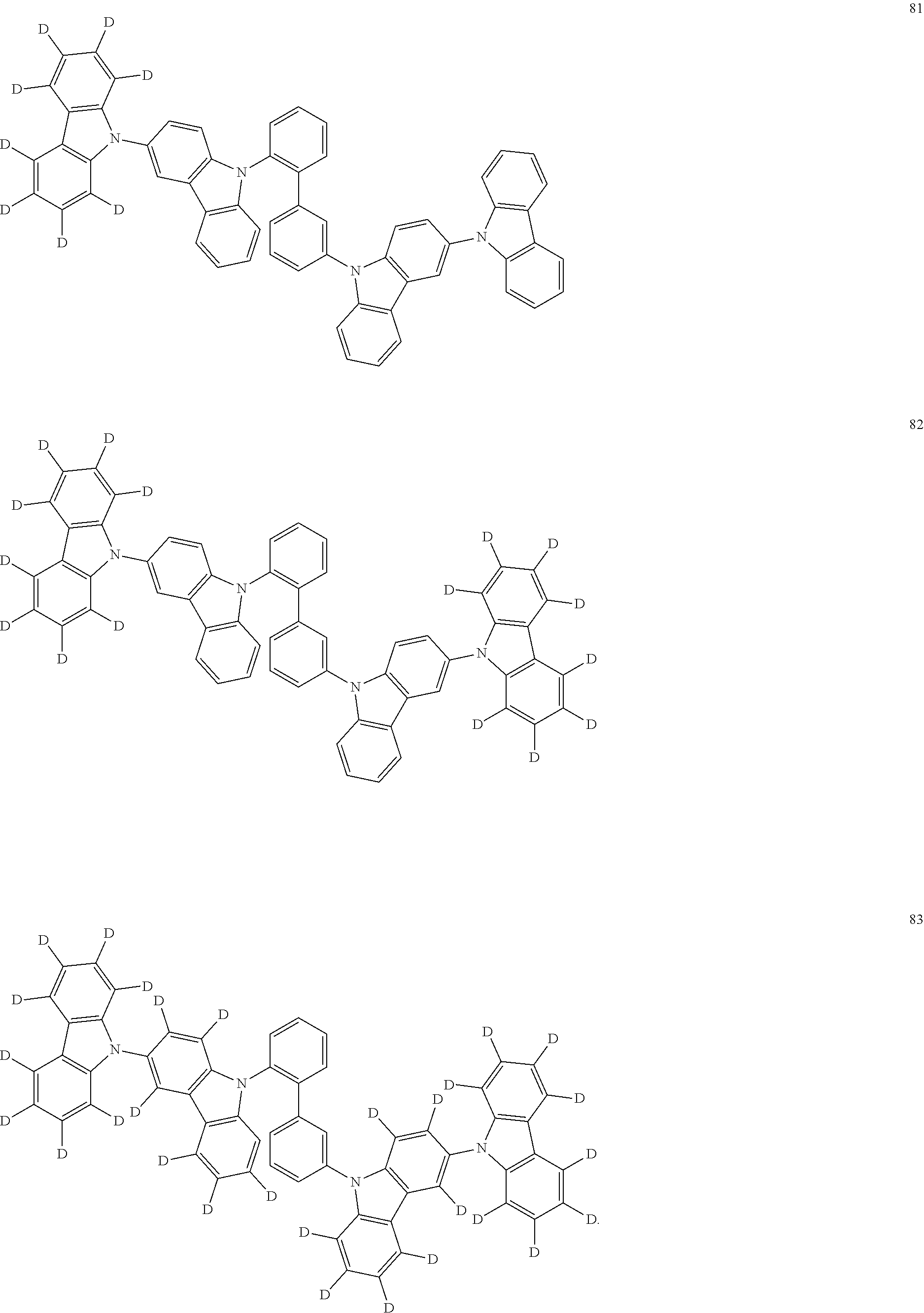

9. An organic electroluminescent element comprising:
a first electrode;
a second electrode facing the first electrode; and
a plurality of functional layers between the first electrode and the second electrode,
wherein at least one of the functional layers comprises a polycyclic compound represented by Formula 1: 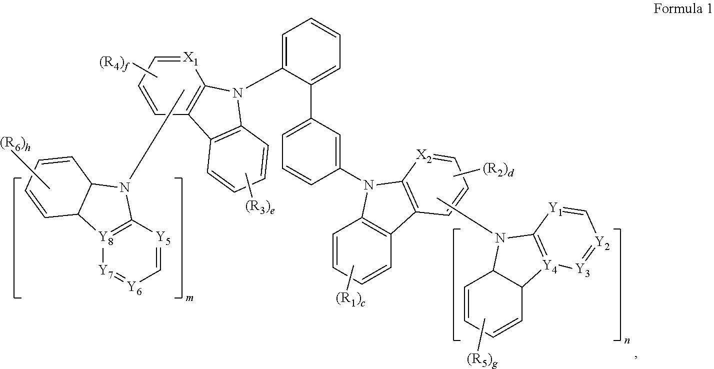
and
wherein, in Formula 1,
“m” and “n” are each independently 0 or 1, and “m+n” is 1 or more,
“c”, “e”, “g”, and “h” are each independently an integer of 0 to 4,
“d” and “f” are each independently an integer of 0 to 3,
X₁ and X₂ are each independently CRₐ or N,
Y₁ to Y₈ are each independently CR_{b} or N, and
Rₐ, R_{b}, and R₁ to R₆ are each independently a hydrogen atom, a deuterium atom, a halogen atom, a cyano group, a substituted or unsubstituted silyl group, a substituted or unsubstituted alkyl group having 1 to 10 carbon atoms, a substituted or unsubstituted ring-forming aryl group having 6 to 30 carbon atoms, or a substituted or unsubstituted ring-forming heteroaryl group having 2 to 30 carbon atoms.

10. The organic electroluminescent element of claim 9, wherein the polycyclic compound represented by Formula 1 is represented by Formula 2-1 or Formula 2-2: 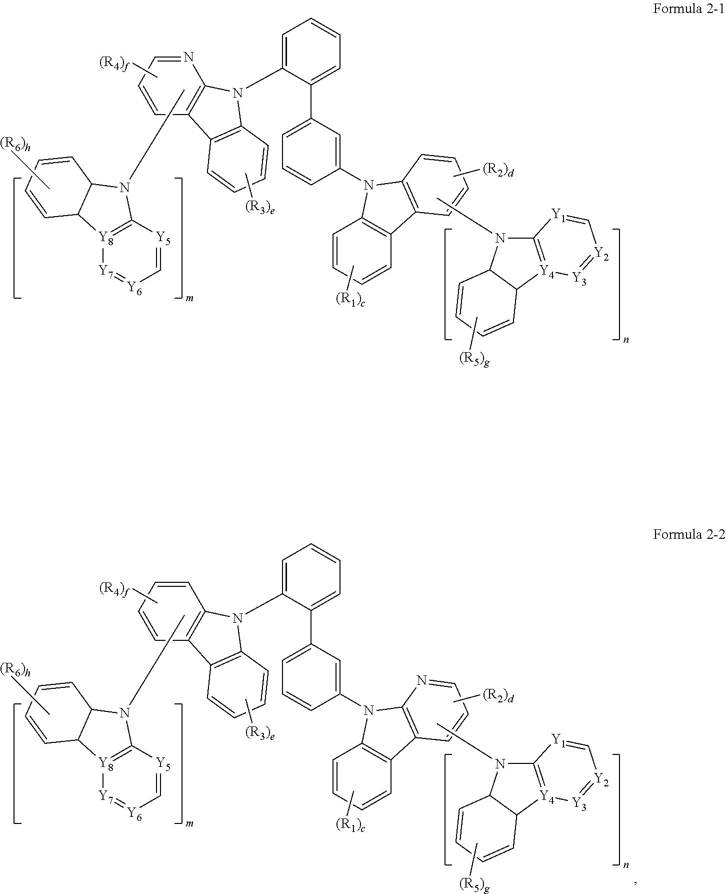
and
wherein, in Formula 2-1 and Formula 2-2, “m”, “n”, “c” to “h”, R₁ to R₆, and Y₁ to Y₈ are each independently the same as defined in Formula 1.

11. The organic electroluminescent element of claim 9, wherein the polycyclic compound represented by Formula 1 is represented by one selected from Formula 3-1 to Formula 3-3: 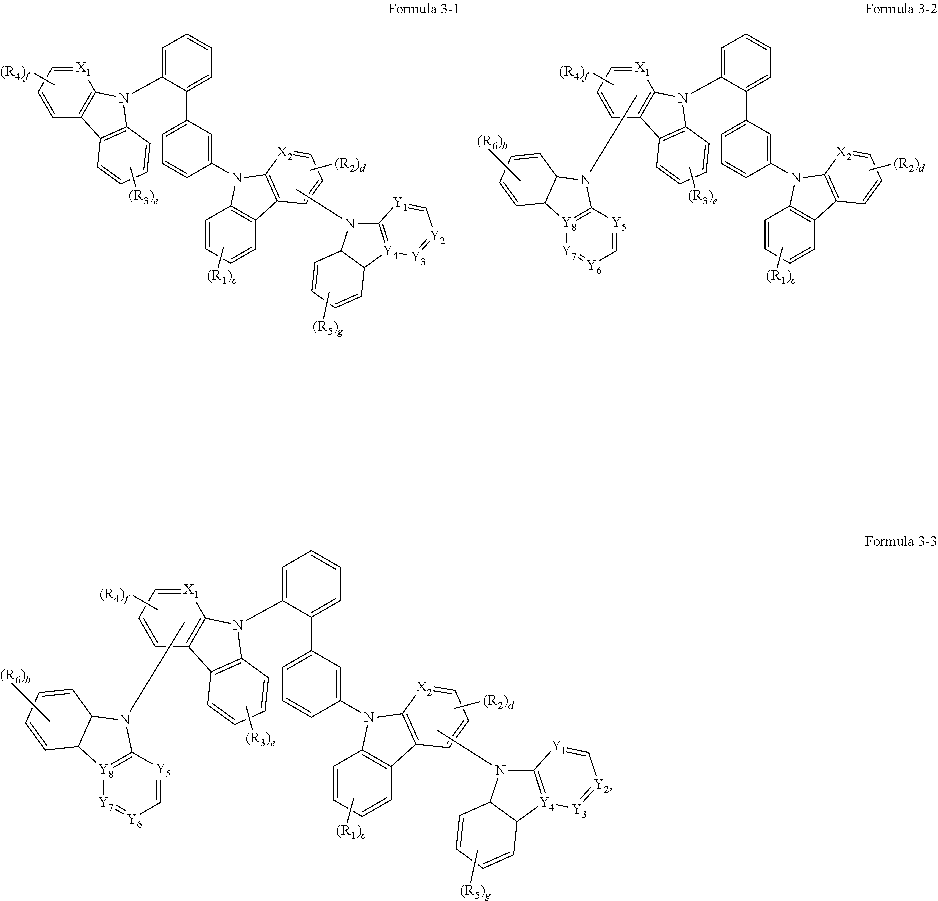
and
wherein, in Formula 3-1 to Formula 3-3, X₁, X₂, R₁ to R₆, Y₁ to Y₈, and “c” to “h” are each independently the same as defined in Formula 1.

12. The organic electroluminescent element of claim 9, wherein the polycyclic compound represented by Formula 1 is a blue light-emitting material.

13. The organic electroluminescent element of claim 10, wherein the polycyclic compound is to emit phosphorescent light or thermally activated delayed fluorescent light.

14. The organic electroluminescent element of claim 10, wherein the polycyclic compound is represented by one selected from the polycyclic compounds in Compound Group 1:

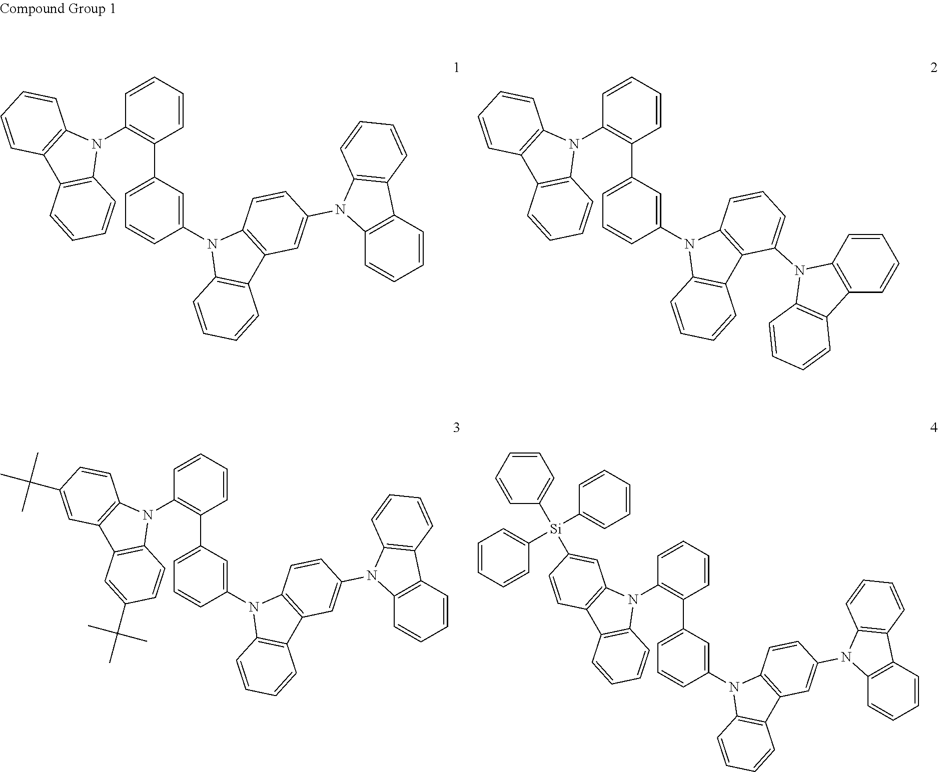

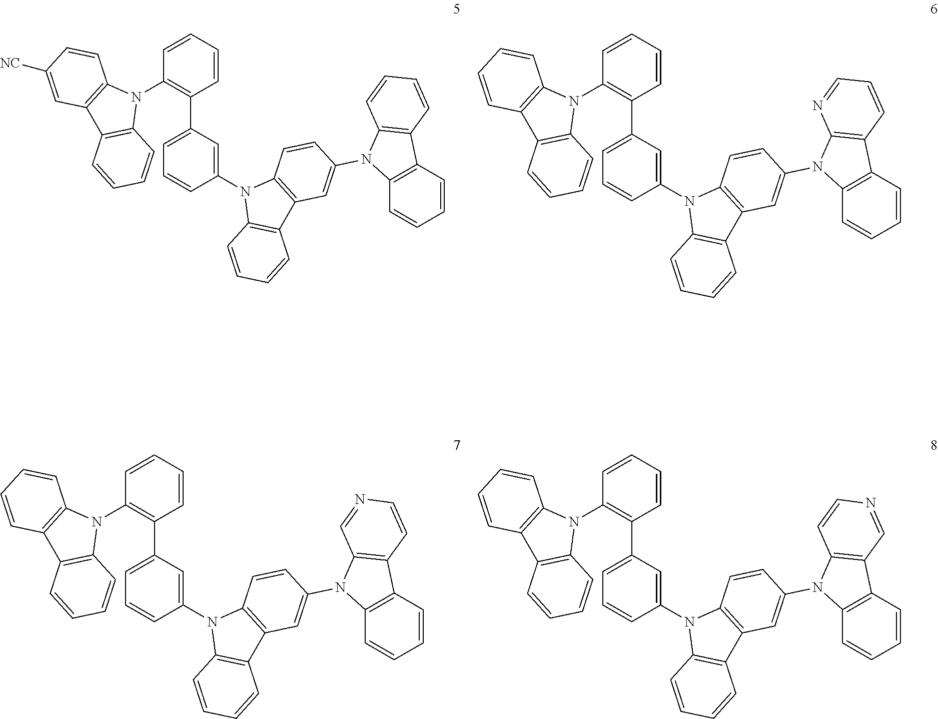 [SMILES: N#Cc1ccc2c(c1)c1ccccc1n2-c1ccccc1-c1cccc(-n2c3ccccc3c3cc(-n4c5ccccc5c5ccccc54)ccc32)c1 ; c1cc(-c2ccccc2-n2c3ccccc3c3ccccc32)cc(-n2c3ccccc3c3cc(-n4c5ccccc5c5cccnc54)ccc32)c1 ; c1cc(-c2ccccc2-n2c3ccccc3c3ccccc32)cc(-n2c3ccccc3c3cc(-n4c5ccccc5c5ccncc54)ccc32)c1 ; c1cc(-c2ccccc2-n2c3ccccc3c3ccccc32)cc(-n2c3ccccc3c3cc(-n4c5ccccc5c5cnccc54)ccc32)c1]

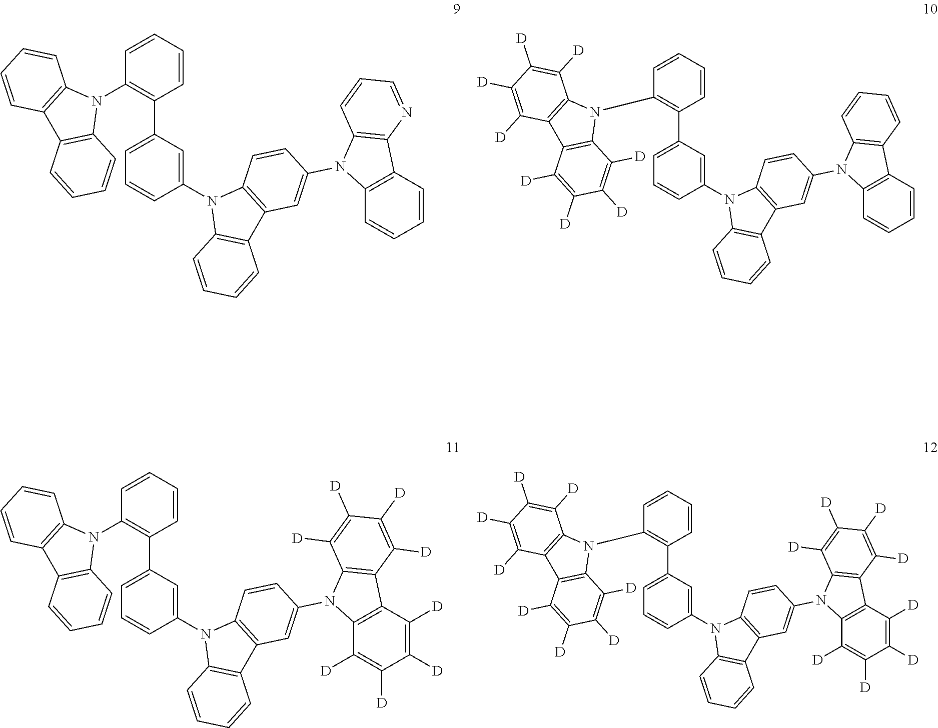

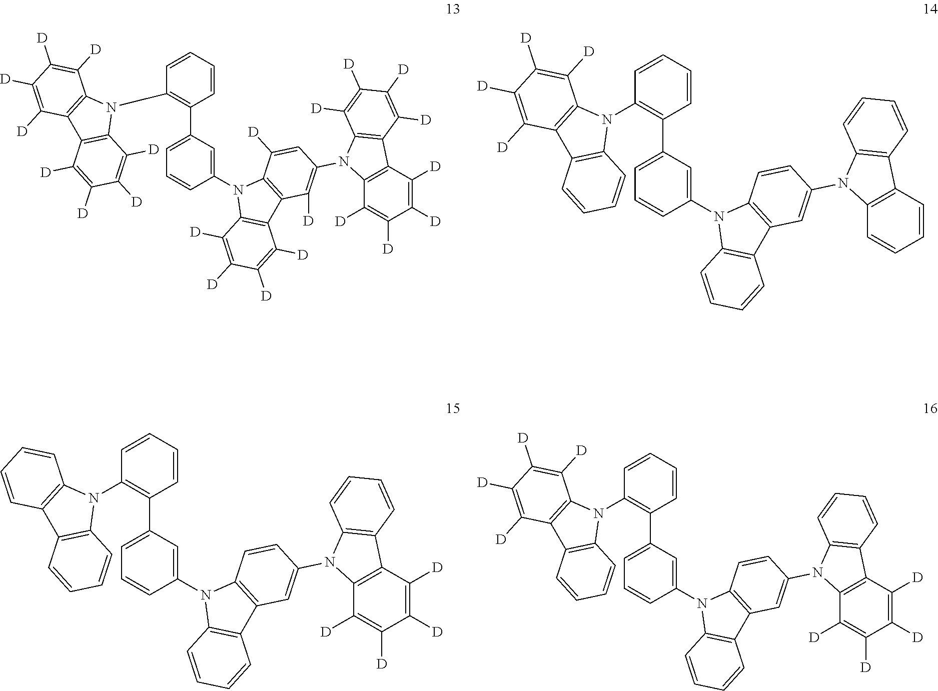

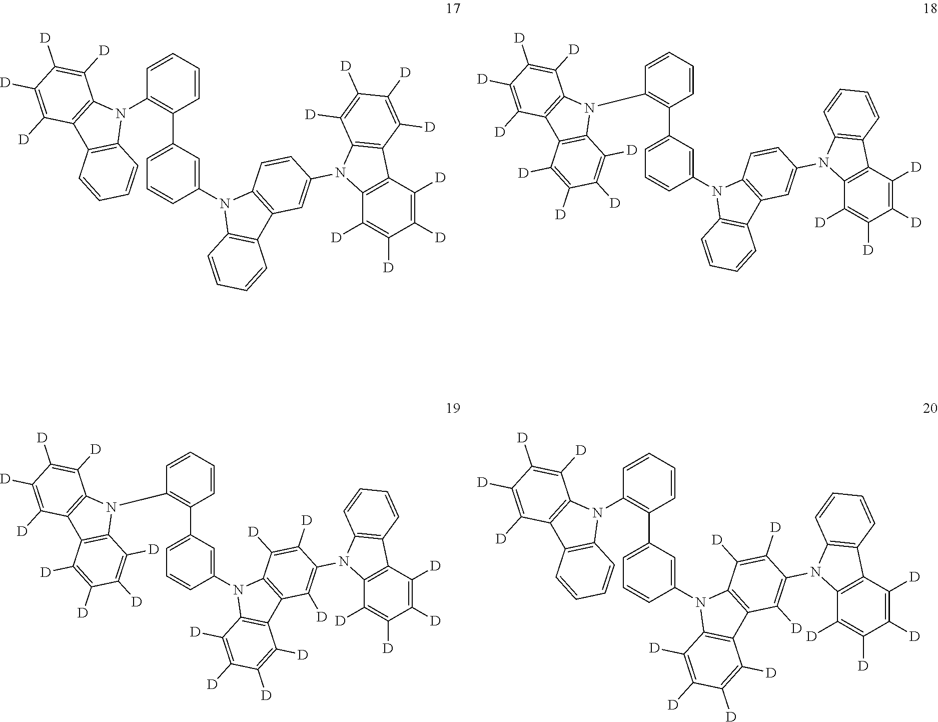

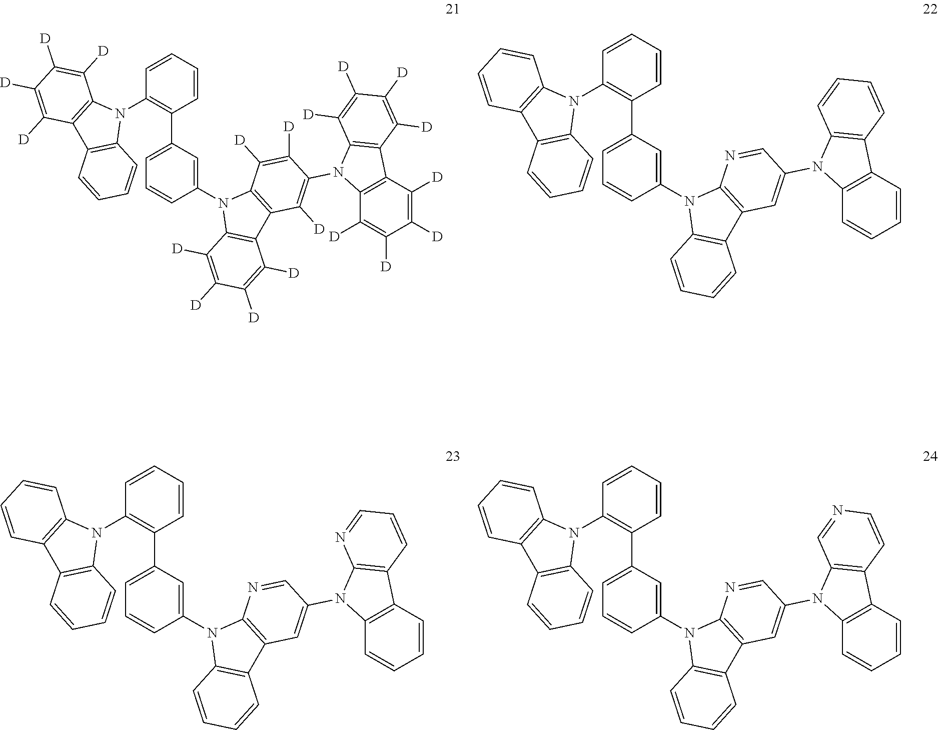 [SMILES: [2H]c1c([2H])c([2H])c2c(c1[2H])c1ccccc1n2-c1ccccc1-c1cccc(-n2c3c([2H])c([2H])c([2H])c([2H])c3c3c([2H])c(-n4c5c([2H])c([2H])c([2H])c([2H])c5c5c([2H])c([2H])c([2H])c([2H])c54)c([2H])c([2H])c32)c1 ; c1cc(-c2ccccc2-n2c3ccccc3c3ccccc32)cc(-n2c3ccccc3c3cc(-n4c5ccccc5c5ccccc54)cnc32)c1 ; c1cc(-c2ccccc2-n2c3ccccc3c3ccccc32)cc(-n2c3ccccc3c3cc(-n4c5ccccc5c5cccnc54)cnc32)c1 ; c1cc(-c2ccccc2-n2c3ccccc3c3ccccc32)cc(-n2c3ccccc3c3cc(-n4c5ccccc5c5ccncc54)cnc32)c1]

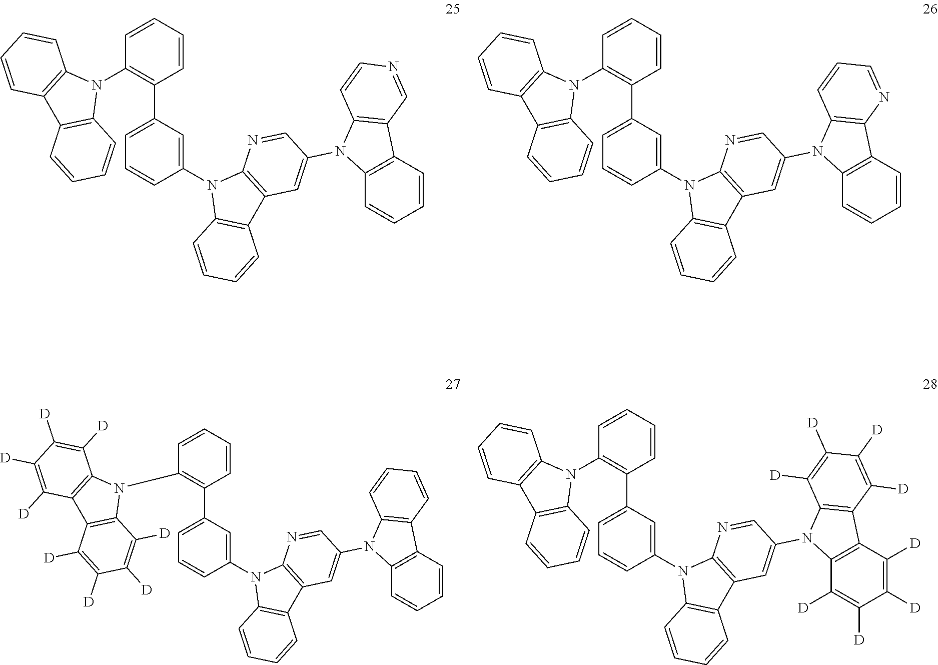 [SMILES: c1cc(-c2ccccc2-n2c3ccccc3c3ccccc32)cc(-n2c3ccccc3c3cc(-n4c5ccccc5c5cnccc54)cnc32)c1 ; c1cc(-c2ccccc2-n2c3ccccc3c3ccccc32)cc(-n2c3ccccc3c3cc(-n4c5ccccc5c5ncccc54)cnc32)c1 ; [2H]c1c([2H])c([2H])c2c(c1[2H])c1c([2H])c([2H])c([2H])c([2H])c1n2-c1ccccc1-c1cccc(-n2c3ccccc3c3cc(-n4c5ccccc5c5ccccc54)cnc32)c1 ; [2H]c1c([2H])c([2H])c2c(c1[2H])c1c([2H])c([2H])c([2H])c([2H])c1n2-c1cnc2c(c1)c1ccccc1n2-c1cccc(-c2ccccc2-n2c3ccccc3c3ccccc32)c1]

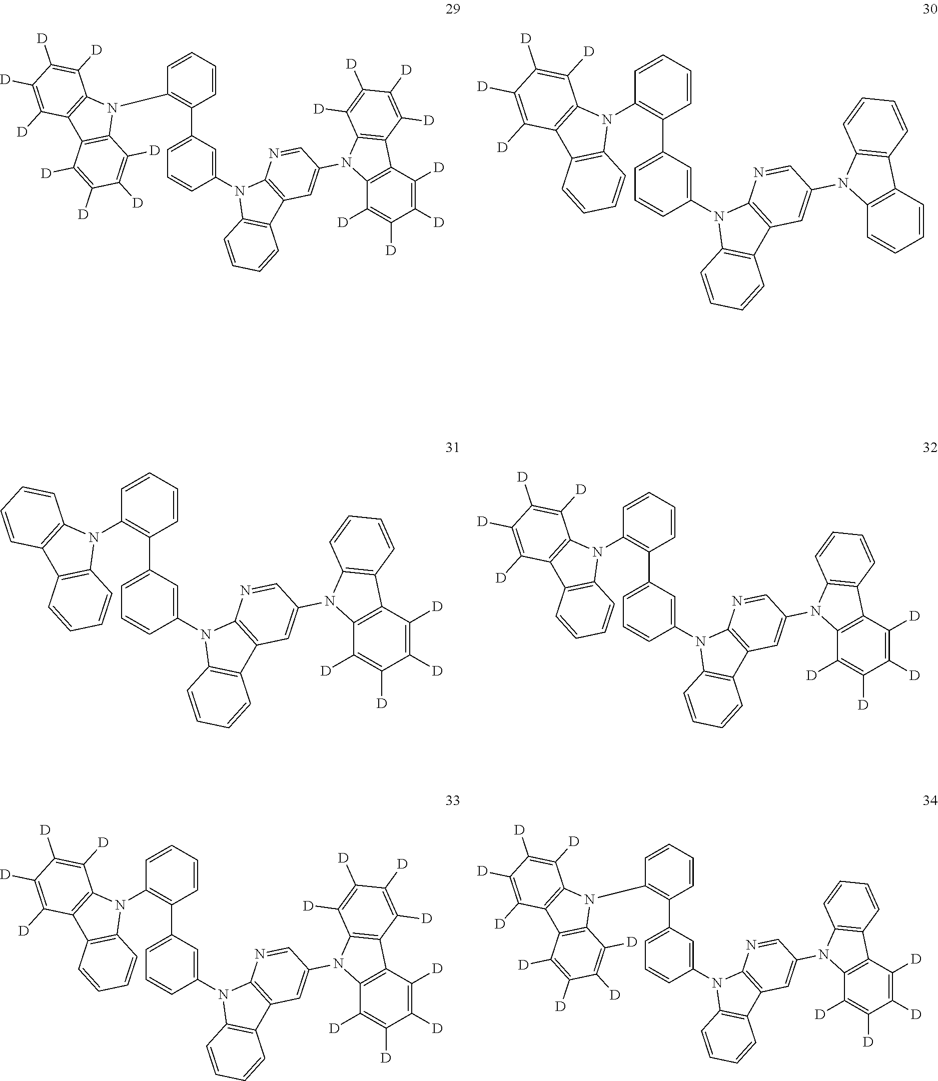

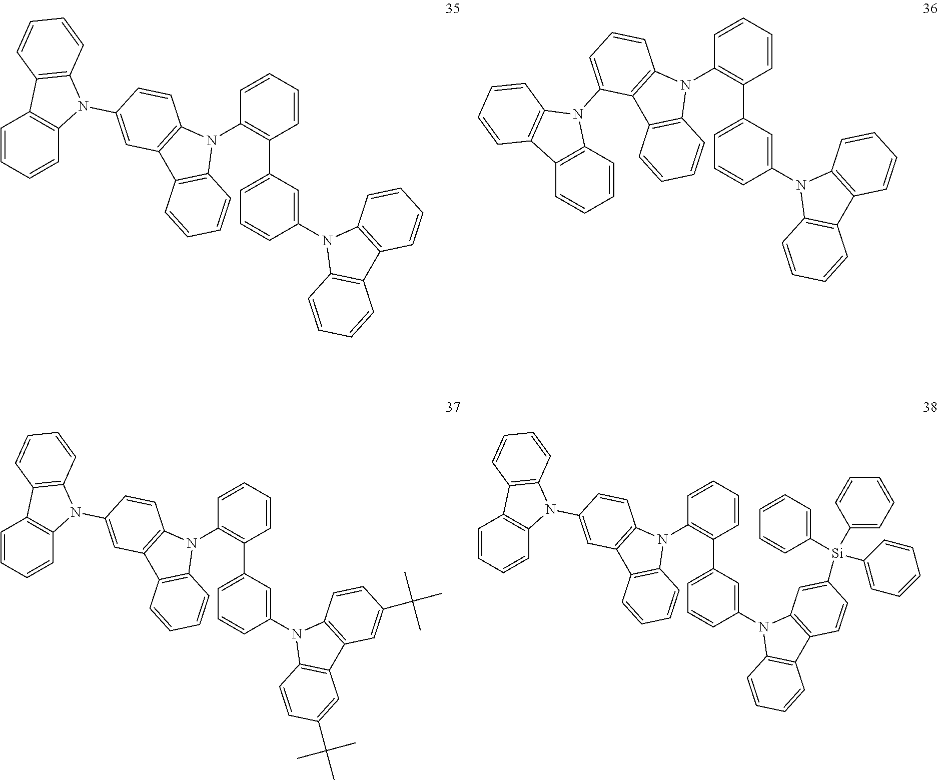 [SMILES: c1cc(-c2ccccc2-n2c3ccccc3c3cc(-n4c5ccccc5c5ccccc54)ccc32)cc(-n2c3ccccc3c3ccccc32)c1 ; c1cc(-c2ccccc2-n2c3ccccc3c3c(-n4c5ccccc5c5ccccc54)cccc32)cc(-n2c3ccccc3c3ccccc32)c1 ; CC(C)(C)c1ccc2c(c1)c1cc(C(C)(C)C)ccc1n2-c1cccc(-c2ccccc2-n2c3ccccc3c3cc(-n4c5ccccc5c5ccccc54)ccc32)c1 ; c1ccc([Si](c2ccccc2)(c2ccccc2)c2ccc3c4ccccc4n(-c4cccc(-c5ccccc5-n5c6ccccc6c6cc(-n7c8ccccc8c8ccccc87)ccc65)c4)c3c2)cc1]

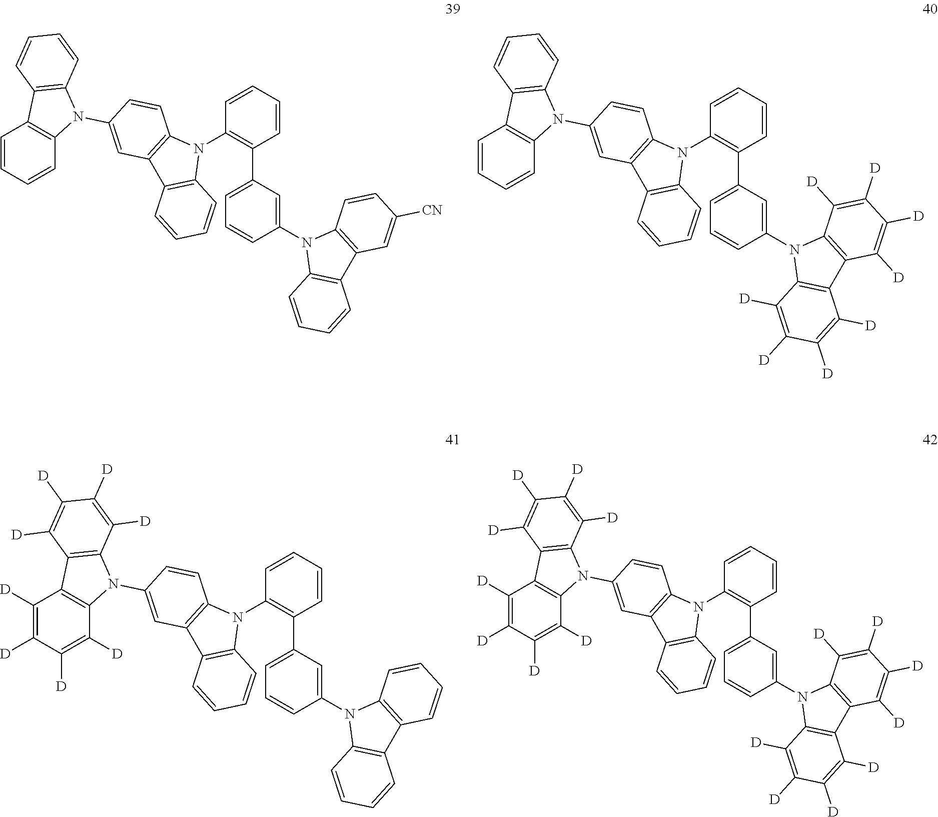

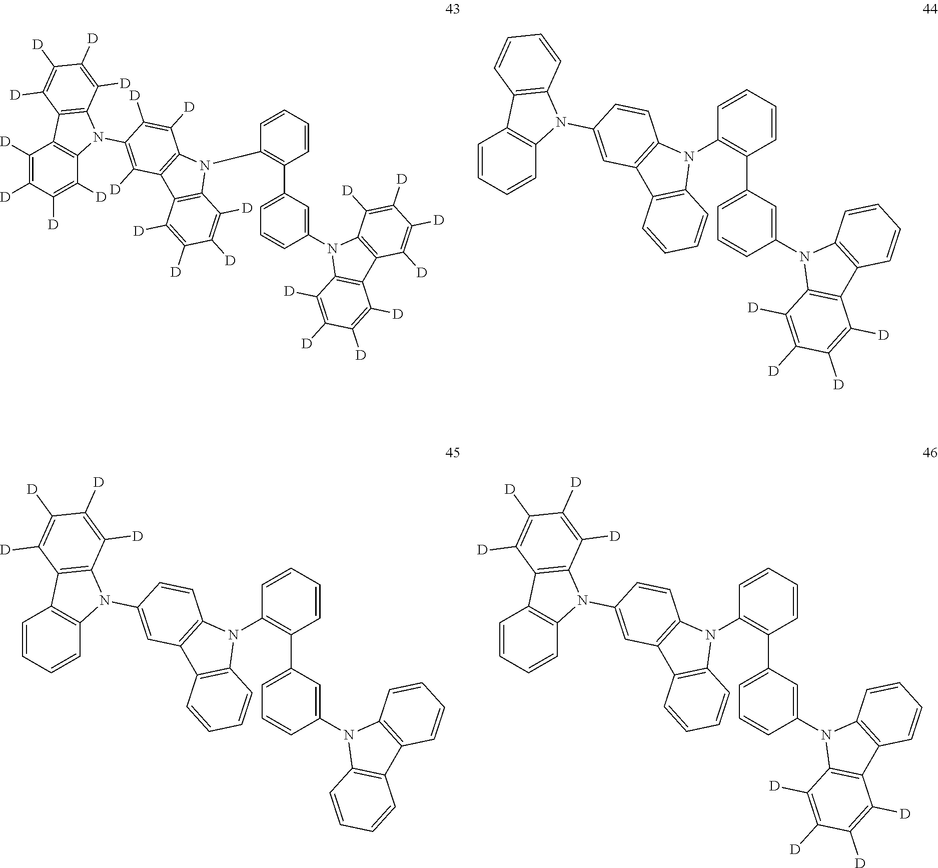

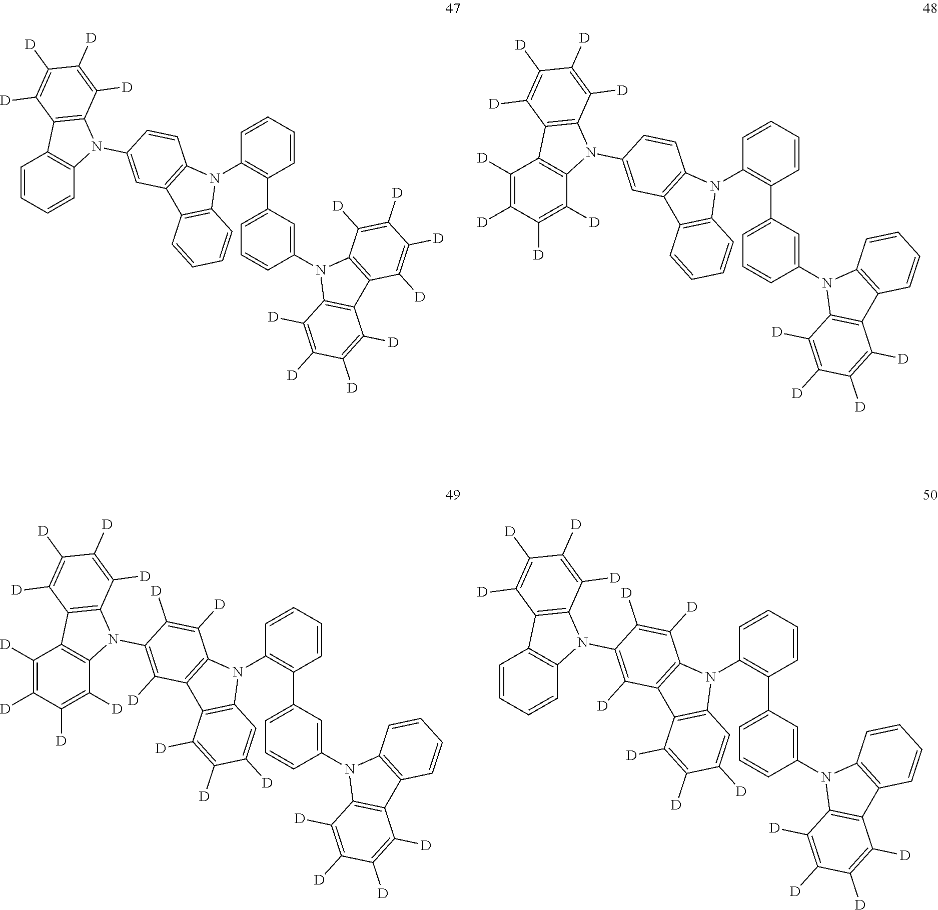

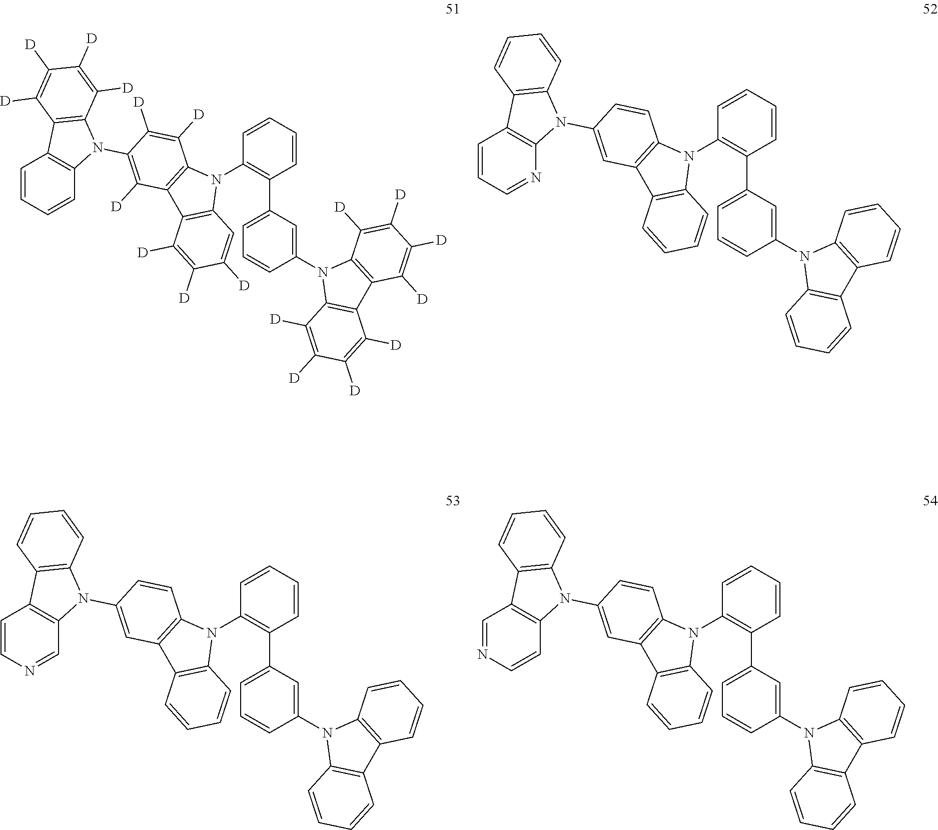 [SMILES: [2H]c1cc2c(c([2H])c1[2H])c1c([2H])c(-n3c4ccccc4c4c([2H])c([2H])c([2H])c([2H])c43)c([2H])c([2H])c1n2-c1ccccc1-c1cccc(-n2c3c([2H])c([2H])c([2H])c([2H])c3c3c([2H])c([2H])c([2H])c([2H])c32)c1 ; c1cc(-c2ccccc2-n2c3ccccc3c3cc(-n4c5ccccc5c5cccnc54)ccc32)cc(-n2c3ccccc3c3ccccc32)c1 ; c1cc(-c2ccccc2-n2c3ccccc3c3cc(-n4c5ccccc5c5ccncc54)ccc32)cc(-n2c3ccccc3c3ccccc32)c1 ; c1cc(-c2ccccc2-n2c3ccccc3c3cc(-n4c5ccccc5c5cnccc54)ccc32)cc(-n2c3ccccc3c3ccccc32)c1]

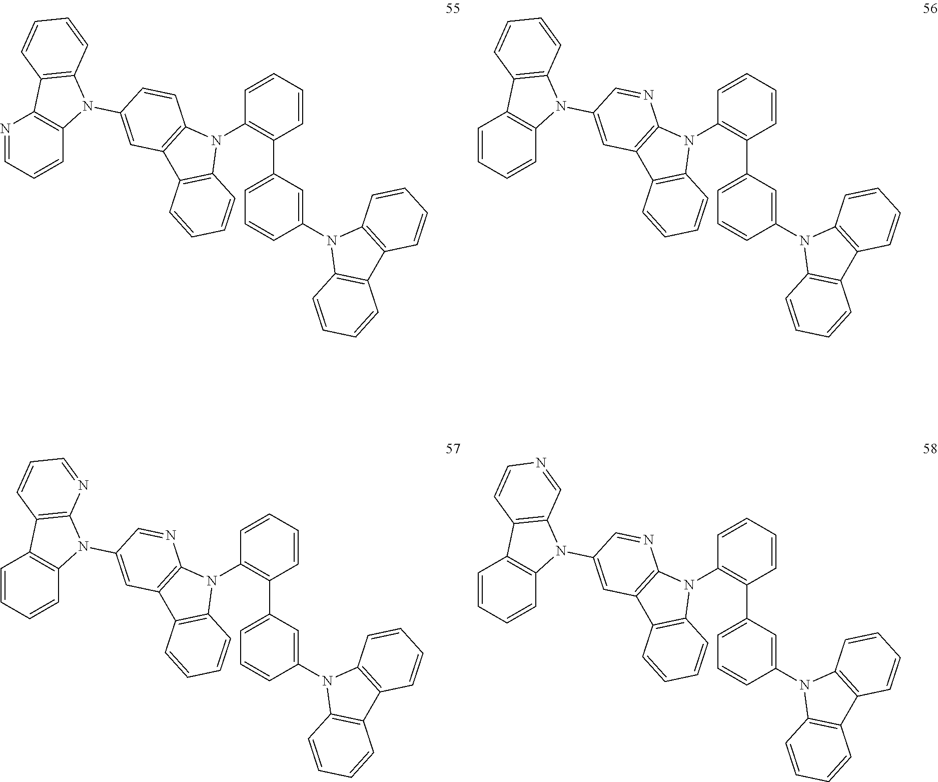 [SMILES: c1cc(-c2ccccc2-n2c3ccccc3c3cc(-n4c5ccccc5c5ncccc54)ccc32)cc(-n2c3ccccc3c3ccccc32)c1 ; c1cc(-c2ccccc2-n2c3ccccc3c3cc(-n4c5ccccc5c5ccccc54)cnc32)cc(-n2c3ccccc3c3ccccc32)c1 ; c1cc(-c2ccccc2-n2c3ccccc3c3cc(-n4c5ccccc5c5cccnc54)cnc32)cc(-n2c3ccccc3c3ccccc32)c1 ; c1cc(-c2ccccc2-n2c3ccccc3c3cc(-n4c5ccccc5c5ccncc54)cnc32)cc(-n2c3ccccc3c3ccccc32)c1]

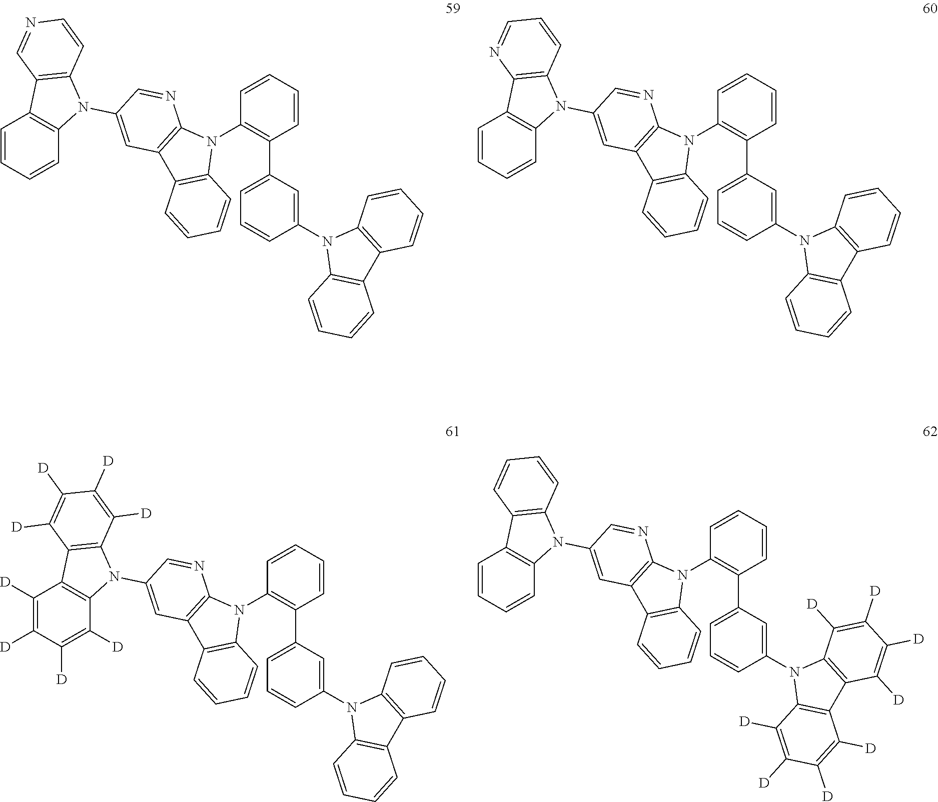 [SMILES: c1cc(-c2ccccc2-n2c3ccccc3c3cc(-n4c5ccccc5c5cnccc54)cnc32)cc(-n2c3ccccc3c3ccccc32)c1 ; c1cc(-c2ccccc2-n2c3ccccc3c3cc(-n4c5ccccc5c5ncccc54)cnc32)cc(-n2c3ccccc3c3ccccc32)c1 ; [2H]c1c([2H])c([2H])c2c(c1[2H])c1c([2H])c([2H])c([2H])c([2H])c1n2-c1cnc2c(c1)c1ccccc1n2-c1ccccc1-c1cccc(-n2c3ccccc3c3ccccc32)c1 ; [2H]c1c([2H])c([2H])c2c(c1[2H])c1c([2H])c([2H])c([2H])c([2H])c1n2-c1cccc(-c2ccccc2-n2c3ccccc3c3cc(-n4c5ccccc5c5ccccc54)cnc32)c1]

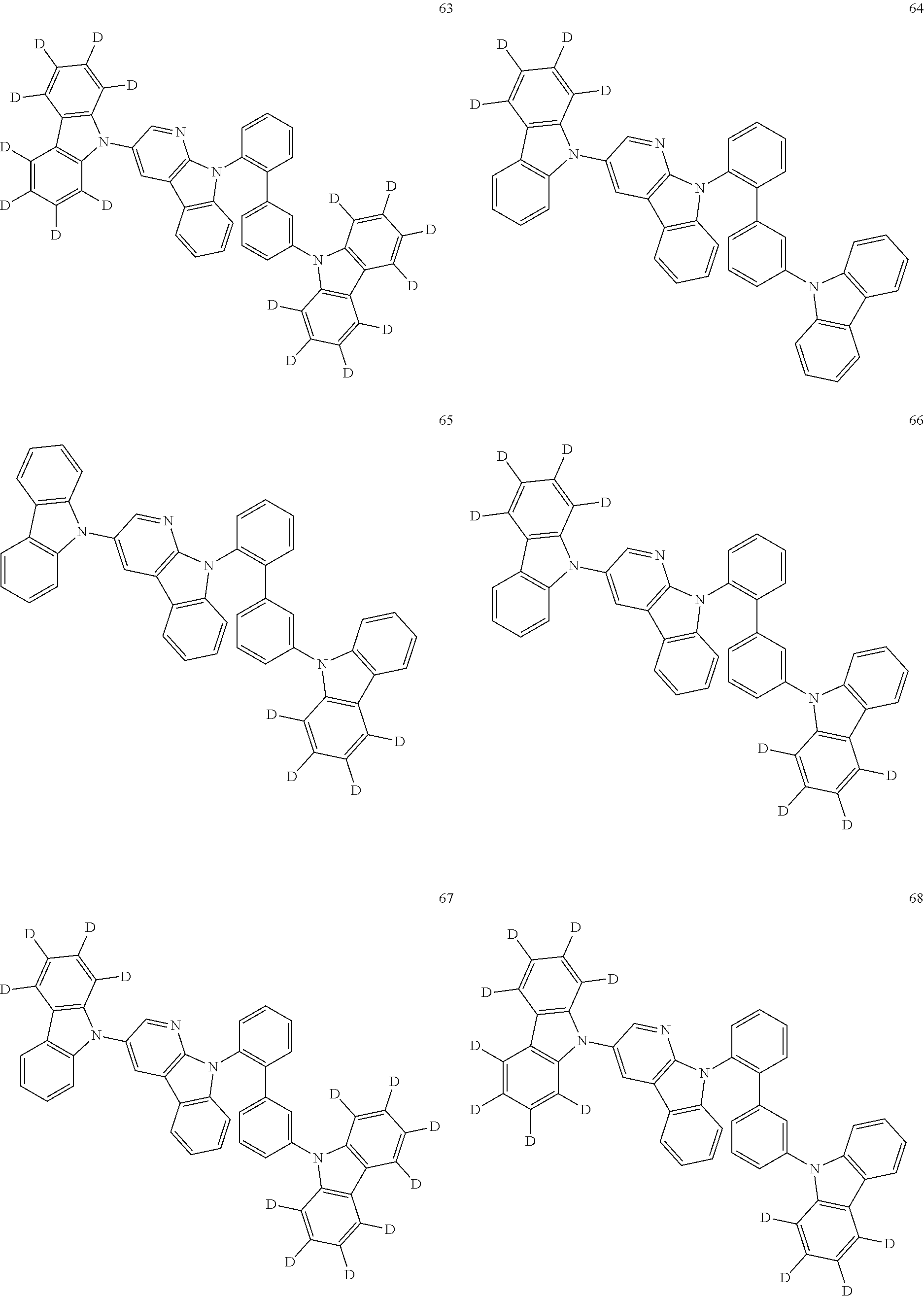

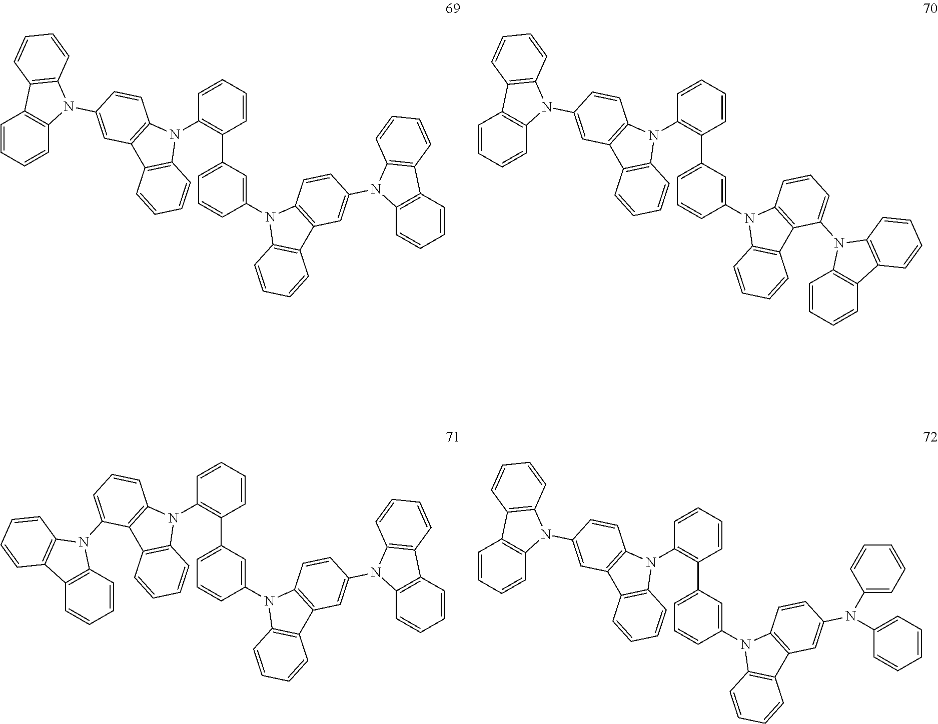

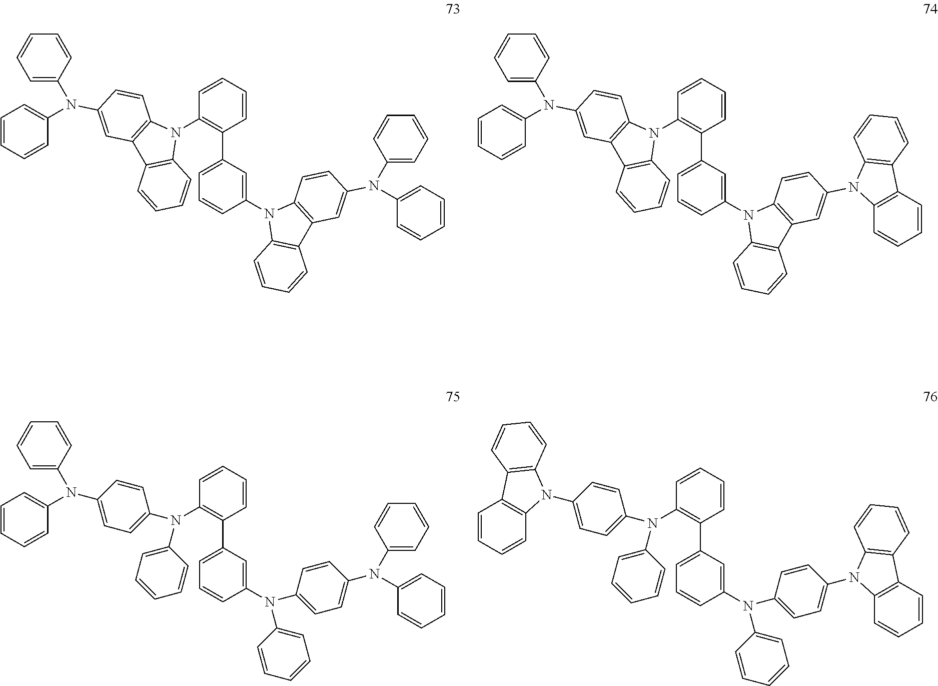 [SMILES: c1ccc(N(c2ccccc2)c2ccc3c(c2)c2ccccc2n3-c2cccc(-c3ccccc3-n3c4ccccc4c4cc(N(c5ccccc5)c5ccccc5)ccc43)c2)cc1 ; c1ccc(N(c2ccccc2)c2ccc3c(c2)c2ccccc2n3-c2ccccc2-c2cccc(-n3c4ccccc4c4cc(-n5c6ccccc6c6ccccc65)ccc43)c2)cc1 ; c1ccc(N(c2ccccc2)c2ccc(N(c3ccccc3)c3cccc(-c4ccccc4N(c4ccccc4)c4ccc(N(c5ccccc5)c5ccccc5)cc4)c3)cc2)cc1 ; c1ccc(N(c2ccc(-n3c4ccccc4c4ccccc43)cc2)c2cccc(-c3ccccc3N(c3ccccc3)c3ccc(-n4c5ccccc5c5ccccc54)cc3)c2)cc1]

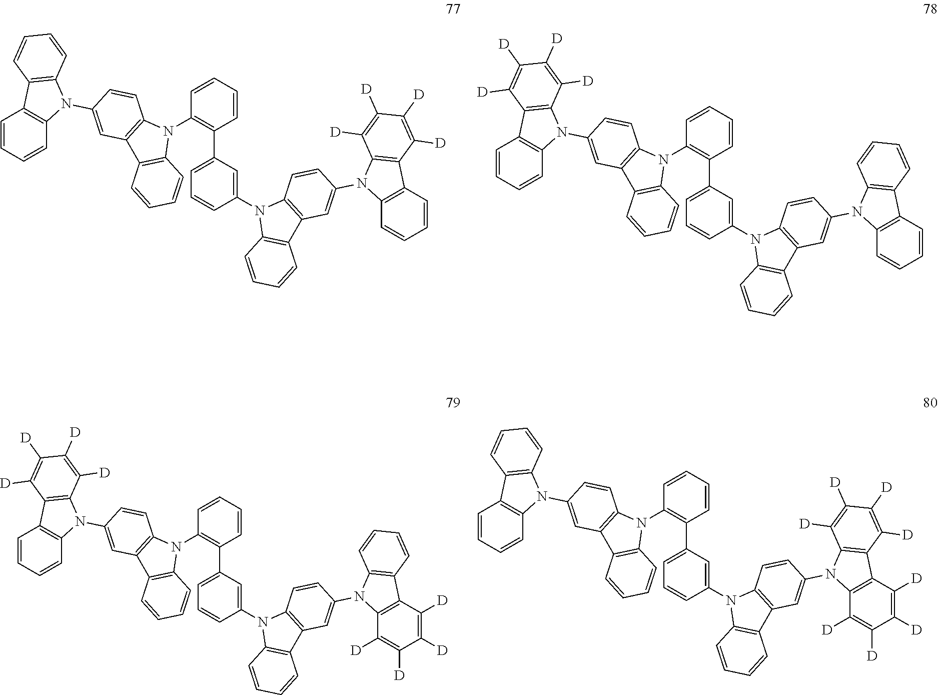

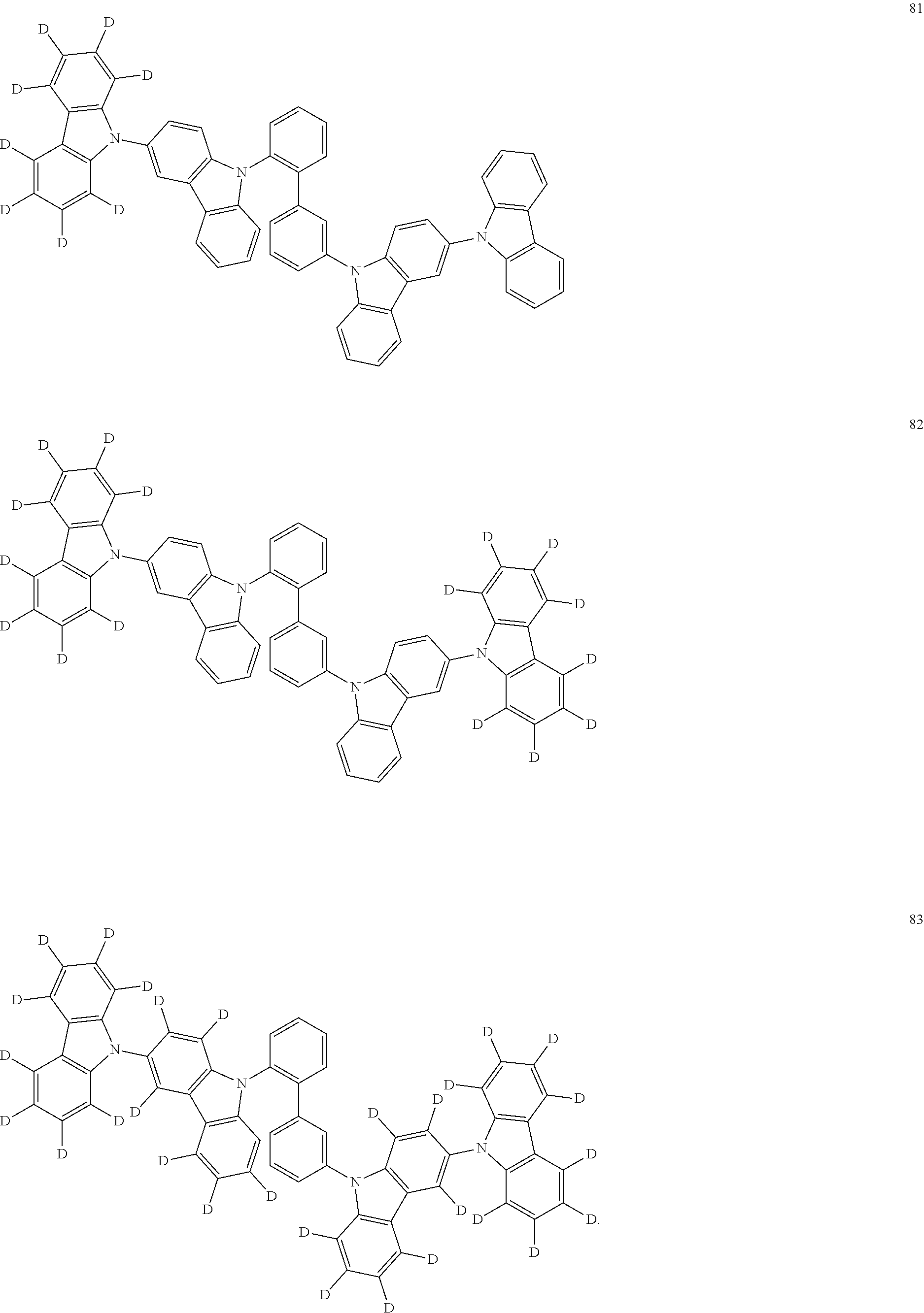

15. A polycyclic compound represented by Formula 1: 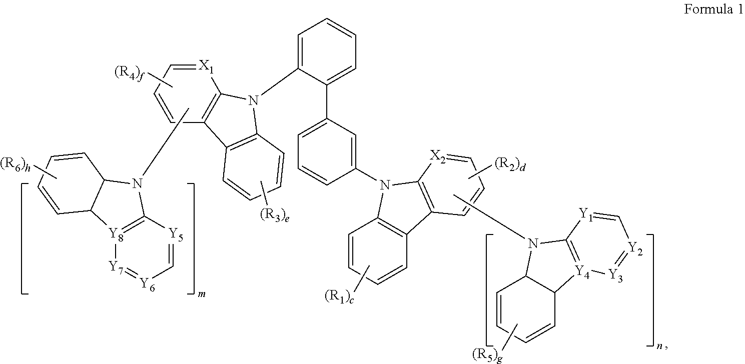
wherein, in Formula 1,
“m” and “n” are each independently 0 or 1, and “m+n” is 1 or more,
X₁ and X₂ are each independently CRₐ or N,
Y₁ to Y₈ are each independently CR_{b} or N,
Rₐ, R_{b}, and R₁ to R₆ are each independently a hydrogen atom, a deuterium atom, a halogen atom, a cyano group, a substituted or unsubstituted silyl group, a substituted or unsubstituted alkyl group having 1 to 10 carbon atoms, a substituted or unsubstituted ring-forming aryl group having 6 to 30 carbon atoms, or a substituted or unsubstituted ring-forming heteroaryl group having 2 to 30 carbon atoms,
“c”, “e”, “g”, and “h” are each independently an integer of 0 to 4, and
“d” and “f” are each independently an integer of 0 to 3.

16. The polycyclic compound of claim 15, wherein the polycyclic compound represented by Formula 1 is represented by Formula 2-1 or Formula 2-2: 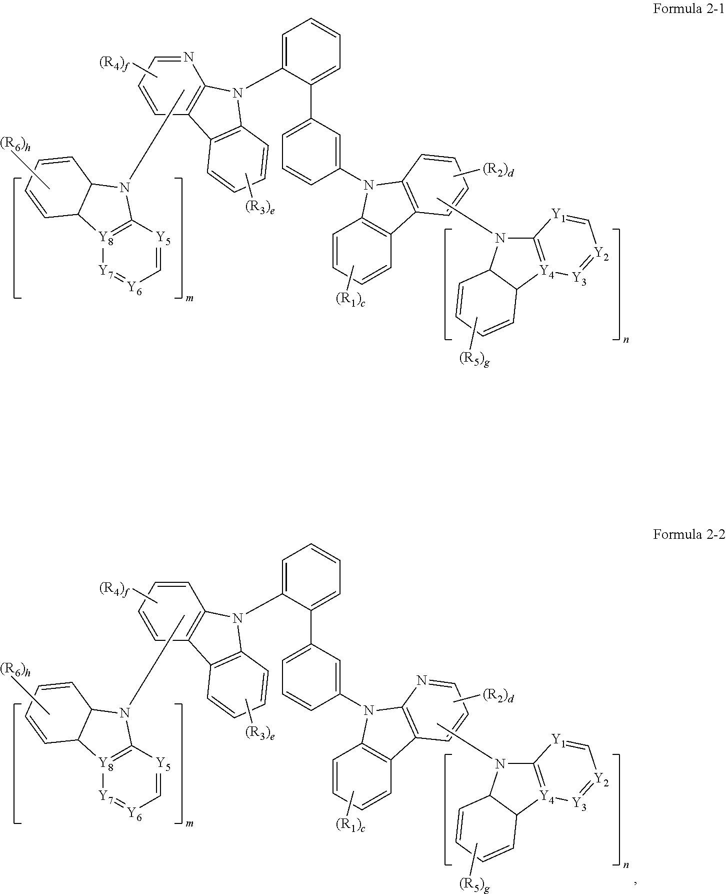
and
wherein, in Formula 2-1 and Formula 2-2, “m”, “n”, R₁ to R₆, “c” to “h”, and Y₁ to Y₈ are each independently the same as defined in Formula 1.

17. The polycyclic compound of claim 15, wherein the polycyclic compound represented by Formula 1 is represented by one selected from Formula 3-1 to Formula 3-3: 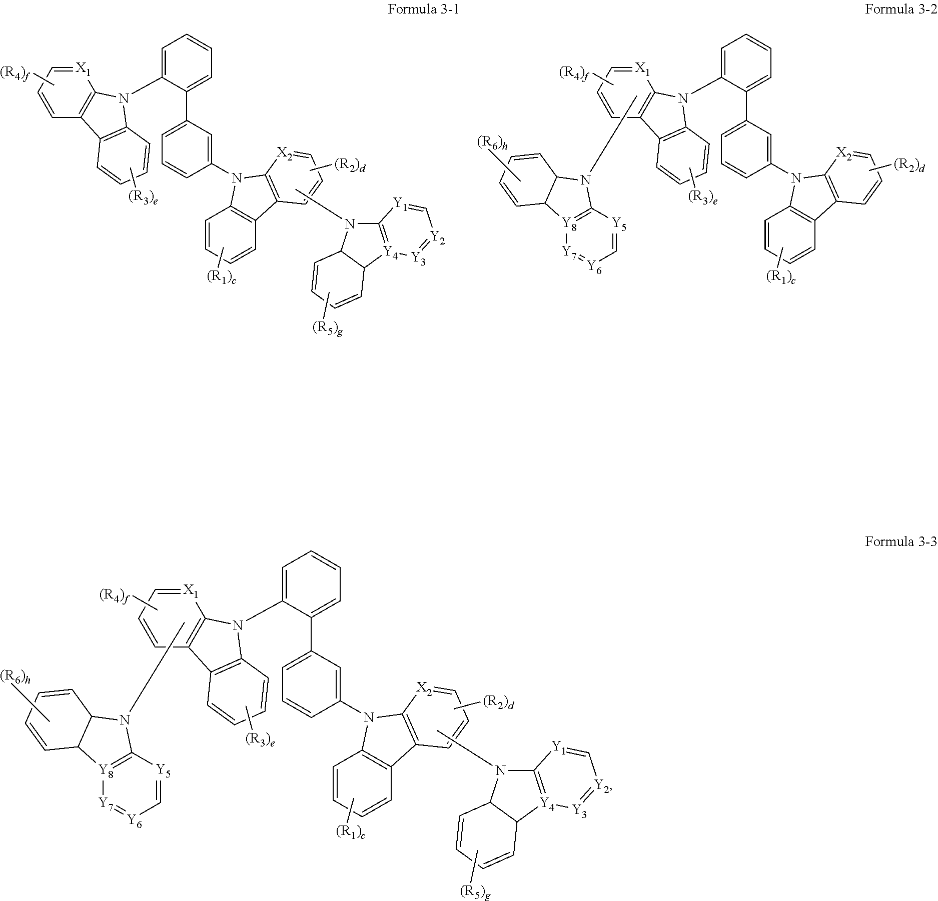
and
wherein, in Formula 3-1 to Formula 3-3, “c” to “h”, X₁, X₂, R₁ to R₆, and Y₁ to Y₈ are each independently the same as defined in Formula 1.

18. The polycyclic compound of claim 15, wherein “c” to “h” are each 1 or more, and at least one selected from R₁ to R₆ is a deuterium atom.

19. The polycyclic compound of claim 15, wherein the polycyclic compound is a blue light-emitting material.

20. The polycyclic compound of claim 15, wherein the polycyclic compound represented by Formula 1 is represented by one selected from the polycyclic compounds represented in Compound Group 1: 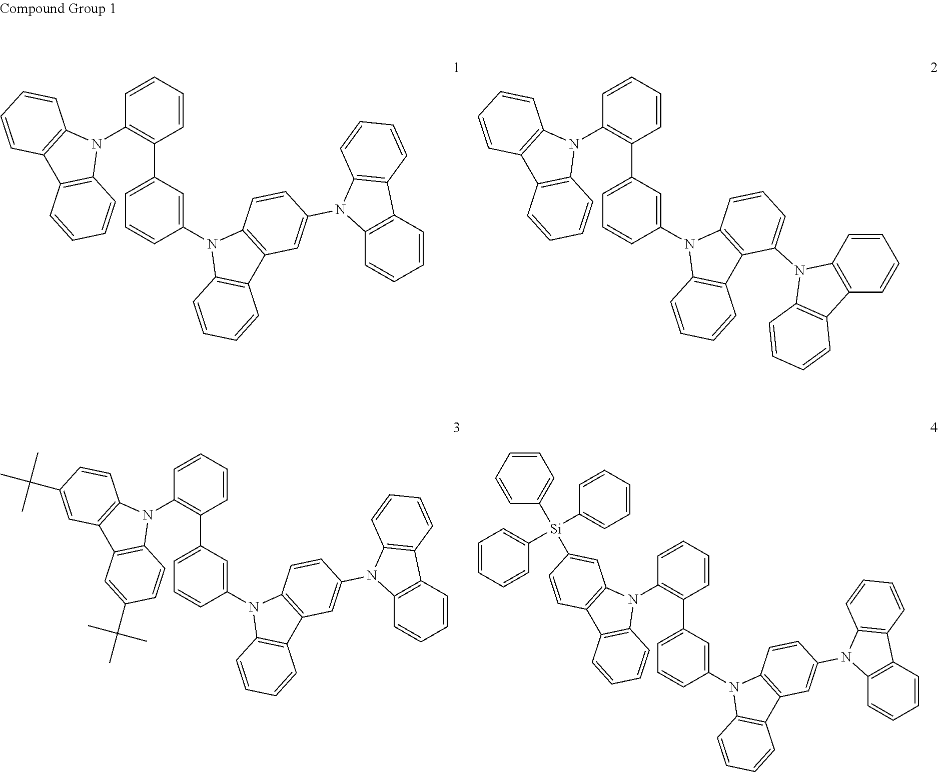 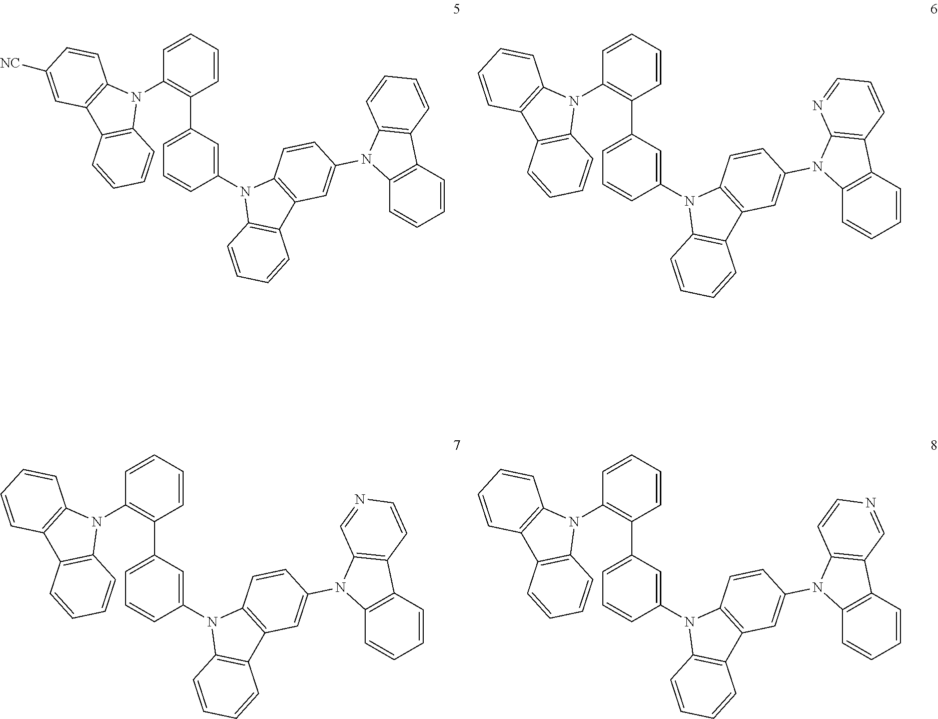 [SMILES: N#Cc1ccc2c(c1)c1ccccc1n2-c1ccccc1-c1cccc(-n2c3ccccc3c3cc(-n4c5ccccc5c5ccccc54)ccc32)c1 ; c1cc(-c2ccccc2-n2c3ccccc3c3ccccc32)cc(-n2c3ccccc3c3cc(-n4c5ccccc5c5cccnc54)ccc32)c1 ; c1cc(-c2ccccc2-n2c3ccccc3c3ccccc32)cc(-n2c3ccccc3c3cc(-n4c5ccccc5c5ccncc54)ccc32)c1 ; c1cc(-c2ccccc2-n2c3ccccc3c3ccccc32)cc(-n2c3ccccc3c3cc(-n4c5ccccc5c5cnccc54)ccc32)c1] 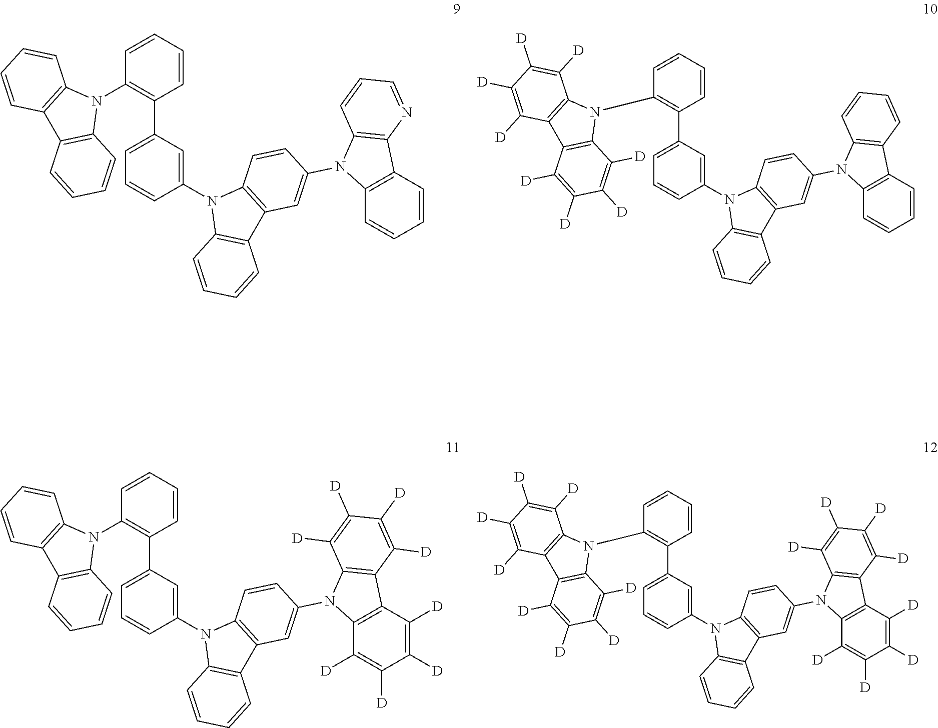 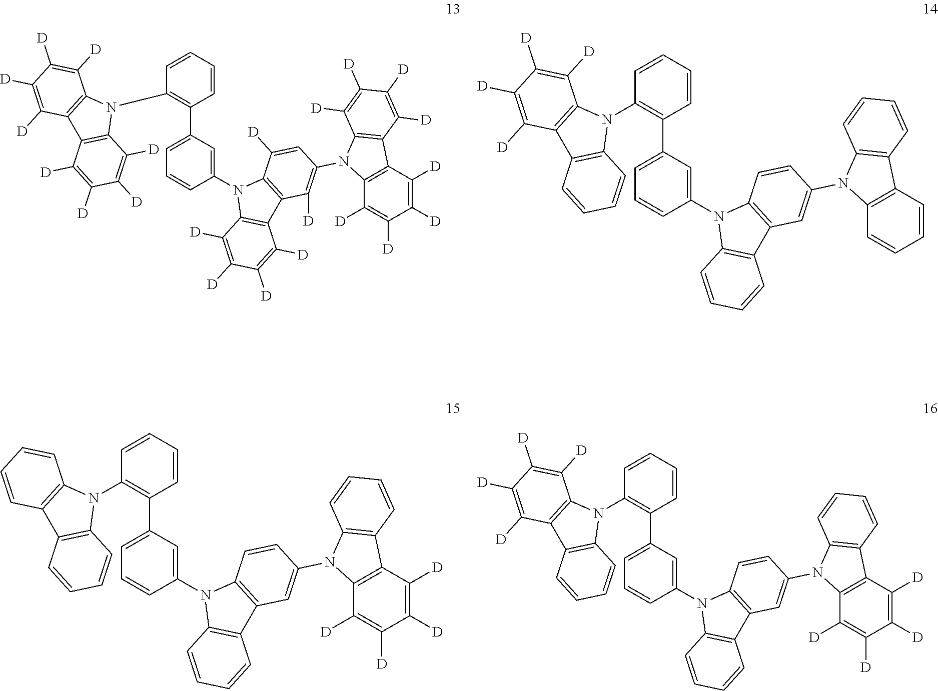 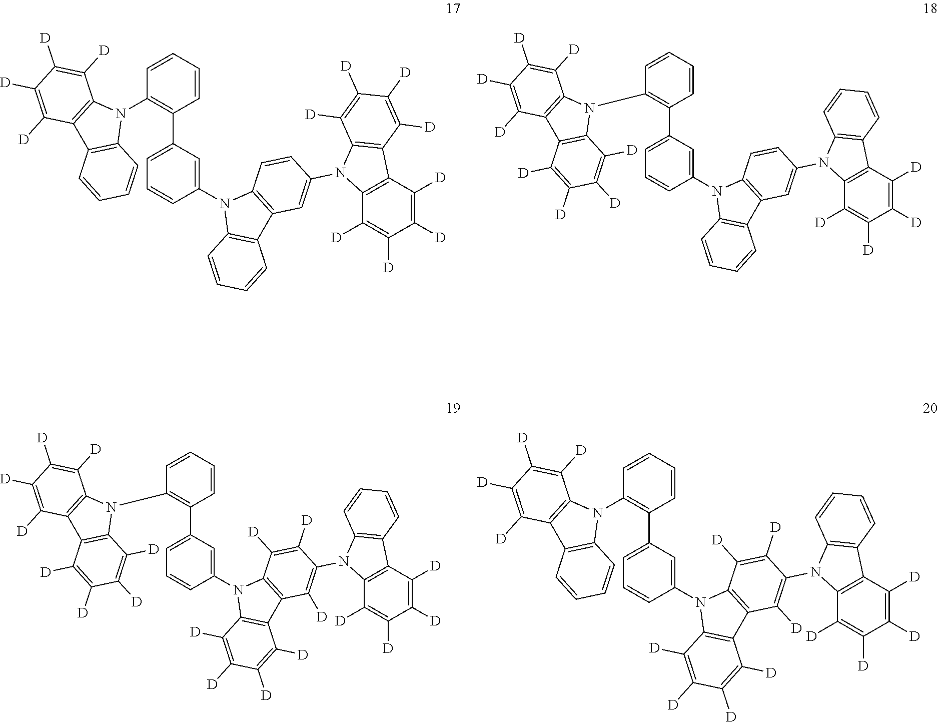 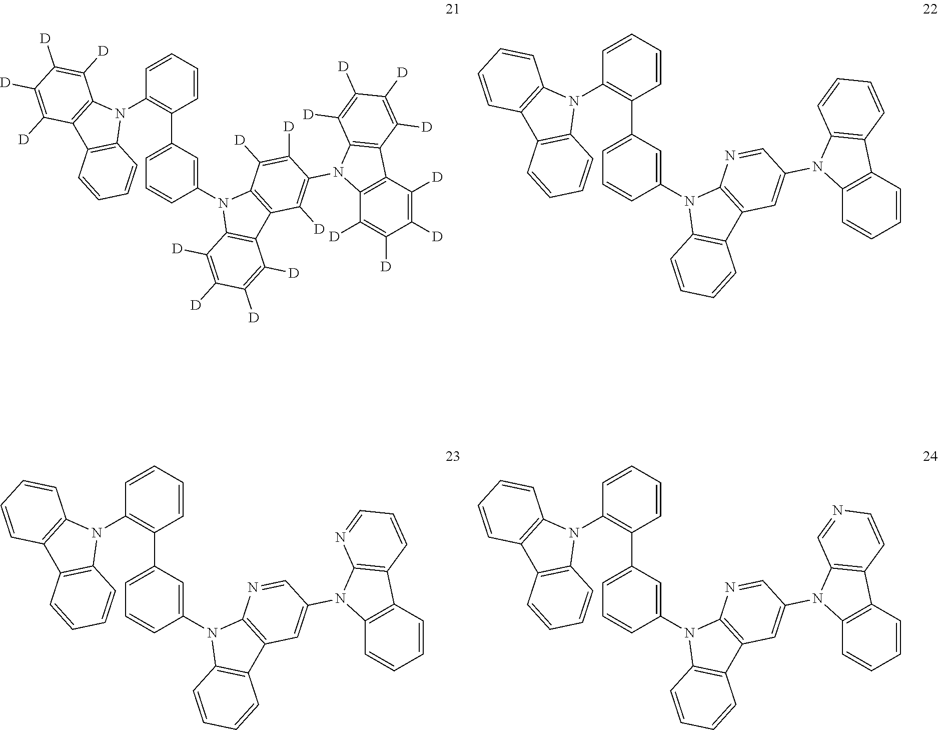 [SMILES: [2H]c1c([2H])c([2H])c2c(c1[2H])c1ccccc1n2-c1ccccc1-c1cccc(-n2c3c([2H])c([2H])c([2H])c([2H])c3c3c([2H])c(-n4c5c([2H])c([2H])c([2H])c([2H])c5c5c([2H])c([2H])c([2H])c([2H])c54)c([2H])c([2H])c32)c1 ; c1cc(-c2ccccc2-n2c3ccccc3c3ccccc32)cc(-n2c3ccccc3c3cc(-n4c5ccccc5c5ccccc54)cnc32)c1 ; c1cc(-c2ccccc2-n2c3ccccc3c3ccccc32)cc(-n2c3ccccc3c3cc(-n4c5ccccc5c5cccnc54)cnc32)c1 ; c1cc(-c2ccccc2-n2c3ccccc3c3ccccc32)cc(-n2c3ccccc3c3cc(-n4c5ccccc5c5ccncc54)cnc32)c1] 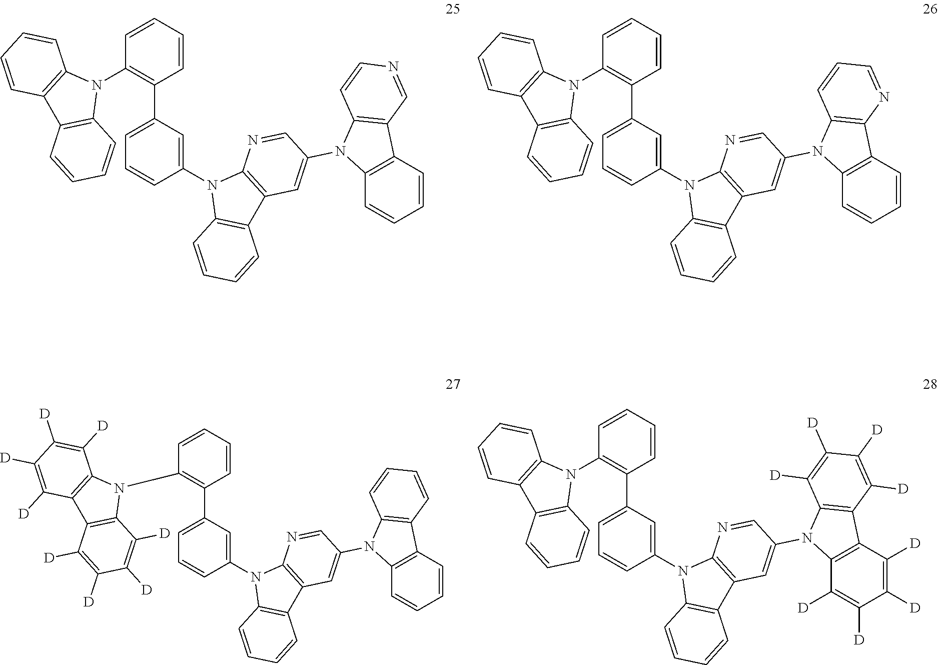 [SMILES: c1cc(-c2ccccc2-n2c3ccccc3c3ccccc32)cc(-n2c3ccccc3c3cc(-n4c5ccccc5c5cnccc54)cnc32)c1 ; c1cc(-c2ccccc2-n2c3ccccc3c3ccccc32)cc(-n2c3ccccc3c3cc(-n4c5ccccc5c5ncccc54)cnc32)c1 ; [2H]c1c([2H])c([2H])c2c(c1[2H])c1c([2H])c([2H])c([2H])c([2H])c1n2-c1ccccc1-c1cccc(-n2c3ccccc3c3cc(-n4c5ccccc5c5ccccc54)cnc32)c1 ; [2H]c1c([2H])c([2H])c2c(c1[2H])c1c([2H])c([2H])c([2H])c([2H])c1n2-c1cnc2c(c1)c1ccccc1n2-c1cccc(-c2ccccc2-n2c3ccccc3c3ccccc32)c1] 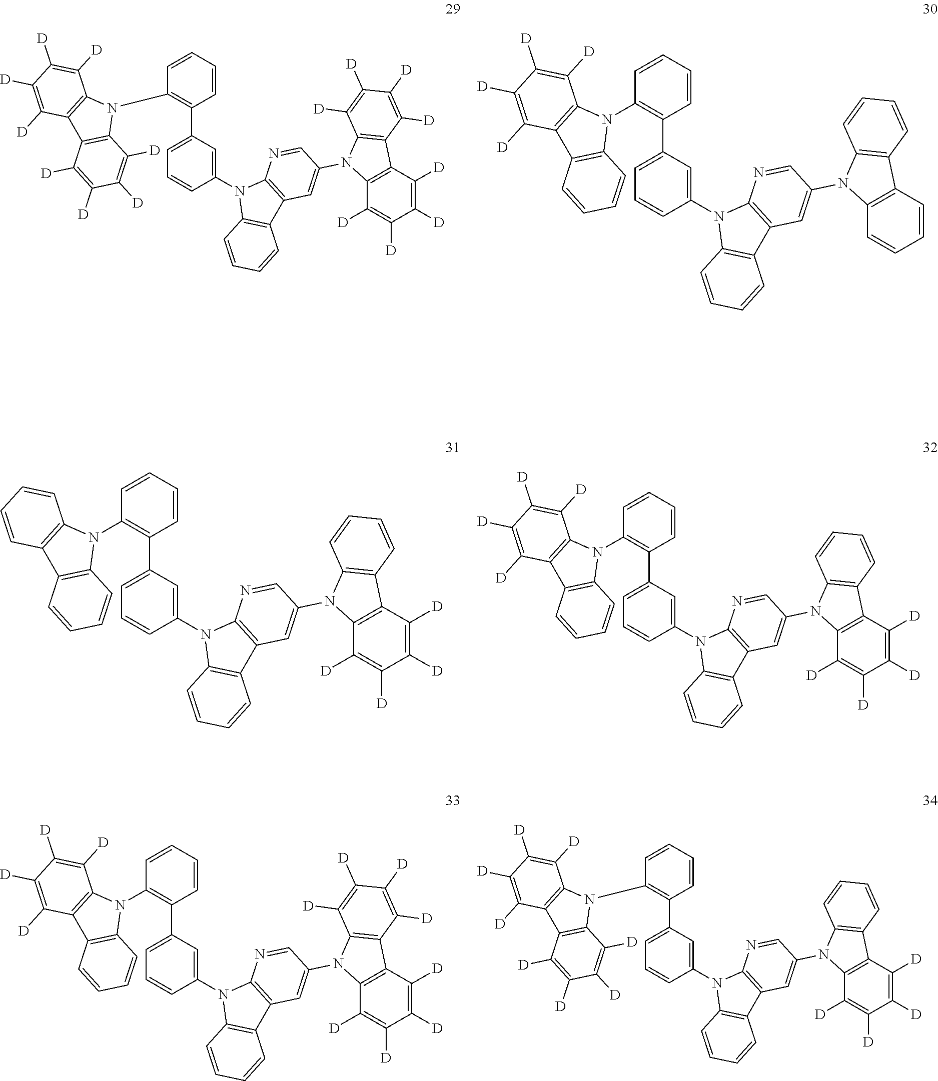 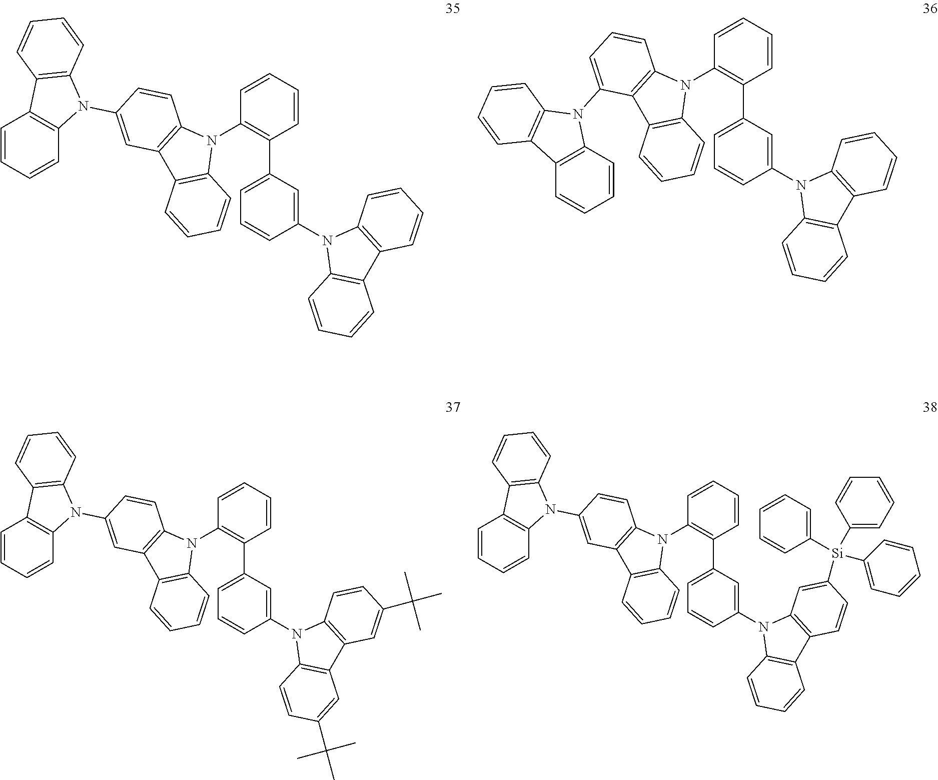 [SMILES: c1cc(-c2ccccc2-n2c3ccccc3c3cc(-n4c5ccccc5c5ccccc54)ccc32)cc(-n2c3ccccc3c3ccccc32)c1 ; c1cc(-c2ccccc2-n2c3ccccc3c3c(-n4c5ccccc5c5ccccc54)cccc32)cc(-n2c3ccccc3c3ccccc32)c1 ; CC(C)(C)c1ccc2c(c1)c1cc(C(C)(C)C)ccc1n2-c1cccc(-c2ccccc2-n2c3ccccc3c3cc(-n4c5ccccc5c5ccccc54)ccc32)c1 ; c1ccc([Si](c2ccccc2)(c2ccccc2)c2ccc3c4ccccc4n(-c4cccc(-c5ccccc5-n5c6ccccc6c6cc(-n7c8ccccc8c8ccccc87)ccc65)c4)c3c2)cc1] 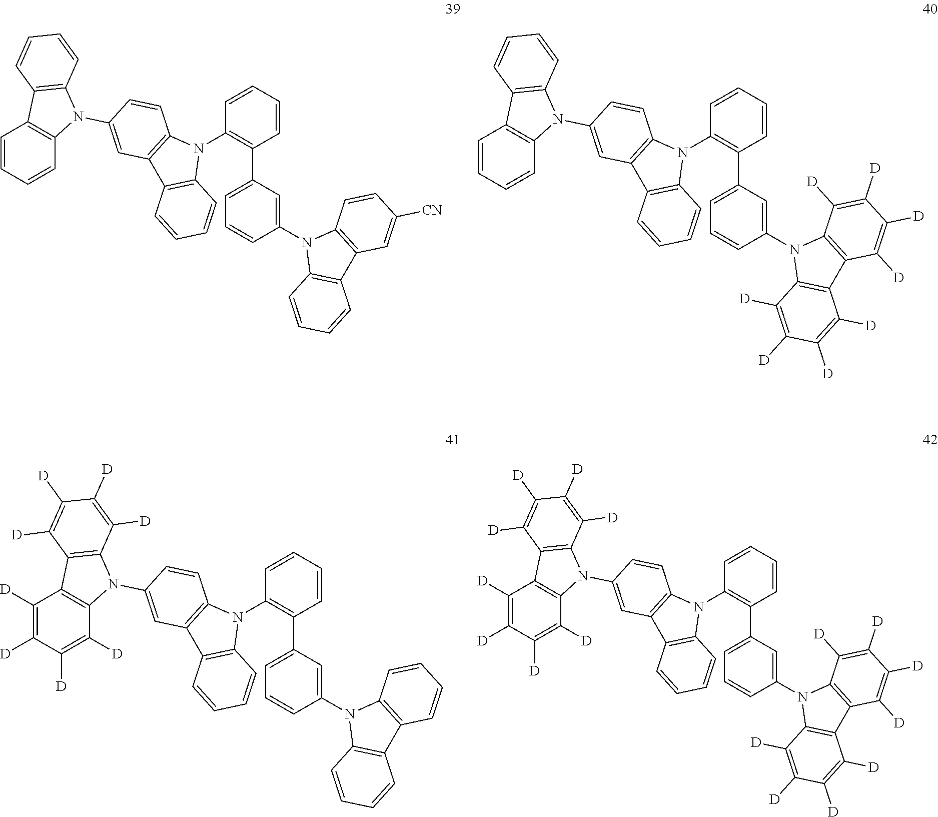 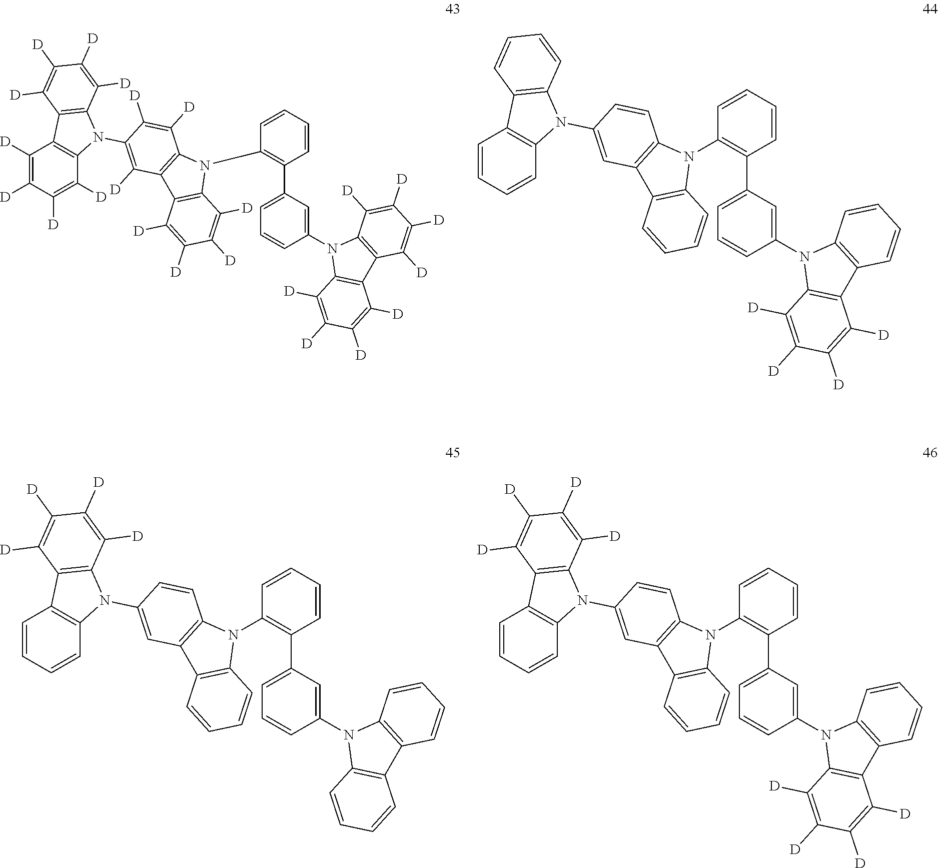 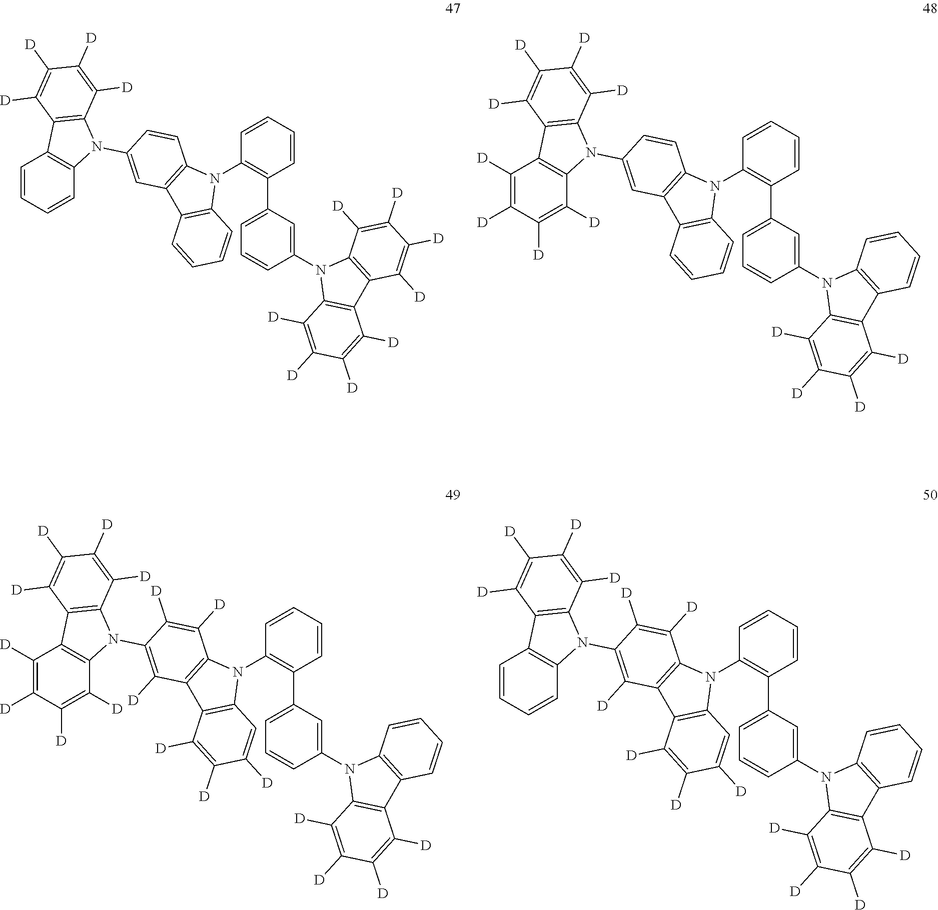 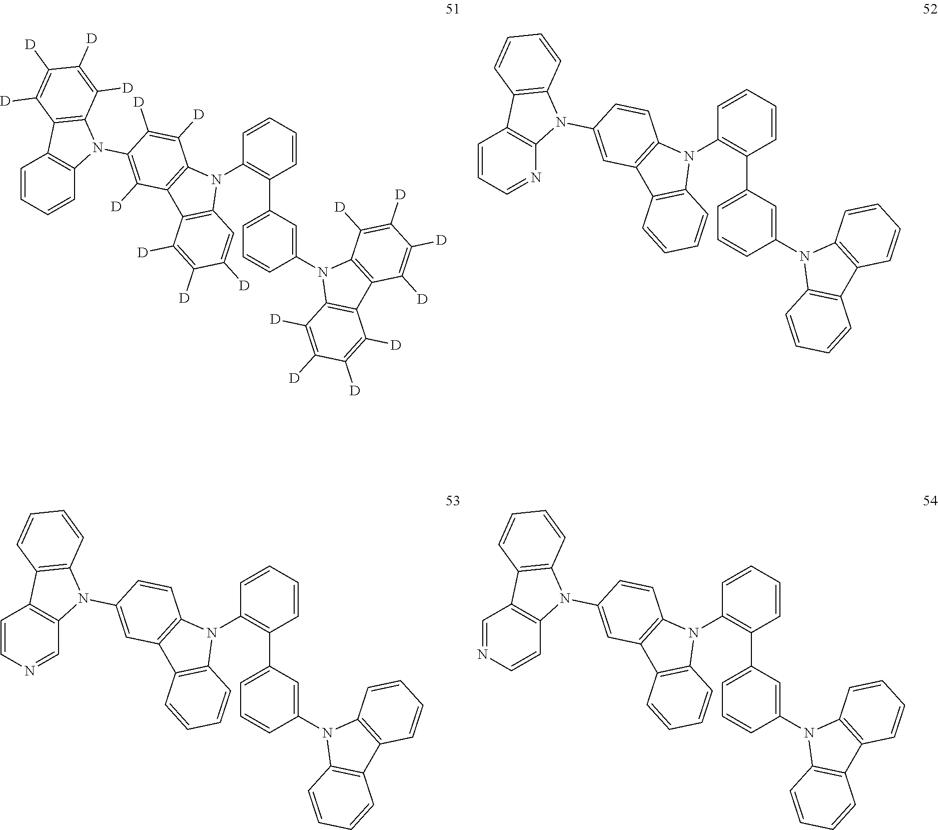 [SMILES: [2H]c1cc2c(c([2H])c1[2H])c1c([2H])c(-n3c4ccccc4c4c([2H])c([2H])c([2H])c([2H])c43)c([2H])c([2H])c1n2-c1ccccc1-c1cccc(-n2c3c([2H])c([2H])c([2H])c([2H])c3c3c([2H])c([2H])c([2H])c([2H])c32)c1 ; c1cc(-c2ccccc2-n2c3ccccc3c3cc(-n4c5ccccc5c5cccnc54)ccc32)cc(-n2c3ccccc3c3ccccc32)c1 ; c1cc(-c2ccccc2-n2c3ccccc3c3cc(-n4c5ccccc5c5ccncc54)ccc32)cc(-n2c3ccccc3c3ccccc32)c1 ; c1cc(-c2ccccc2-n2c3ccccc3c3cc(-n4c5ccccc5c5cnccc54)ccc32)cc(-n2c3ccccc3c3ccccc32)c1] 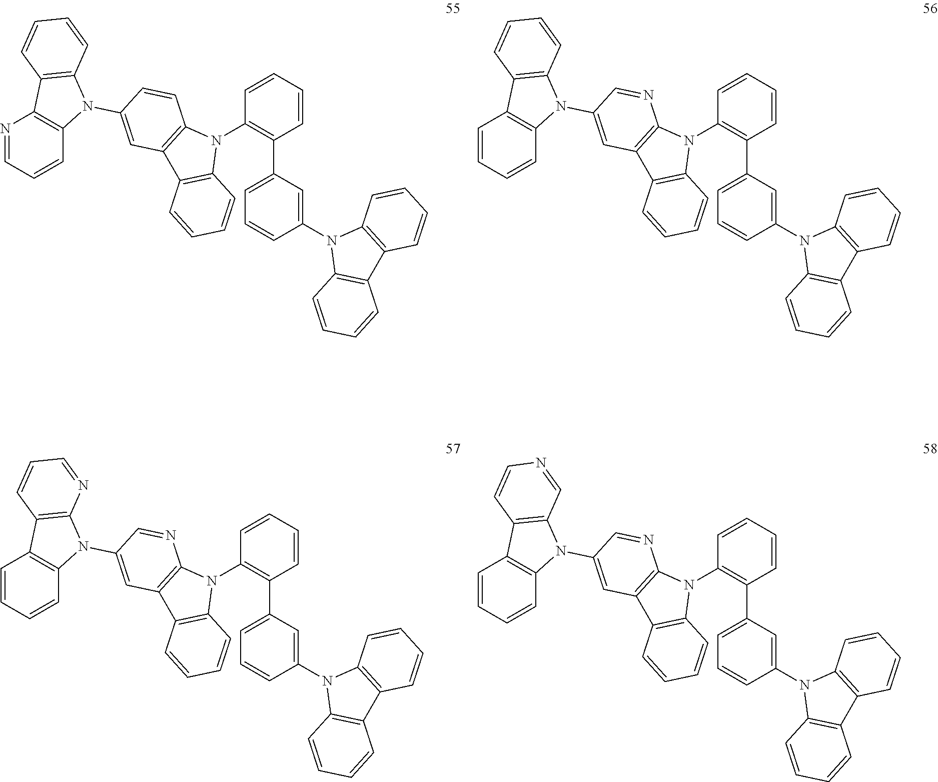 [SMILES: c1cc(-c2ccccc2-n2c3ccccc3c3cc(-n4c5ccccc5c5ncccc54)ccc32)cc(-n2c3ccccc3c3ccccc32)c1 ; c1cc(-c2ccccc2-n2c3ccccc3c3cc(-n4c5ccccc5c5ccccc54)cnc32)cc(-n2c3ccccc3c3ccccc32)c1 ; c1cc(-c2ccccc2-n2c3ccccc3c3cc(-n4c5ccccc5c5cccnc54)cnc32)cc(-n2c3ccccc3c3ccccc32)c1 ; c1cc(-c2ccccc2-n2c3ccccc3c3cc(-n4c5ccccc5c5ccncc54)cnc32)cc(-n2c3ccccc3c3ccccc32)c1] 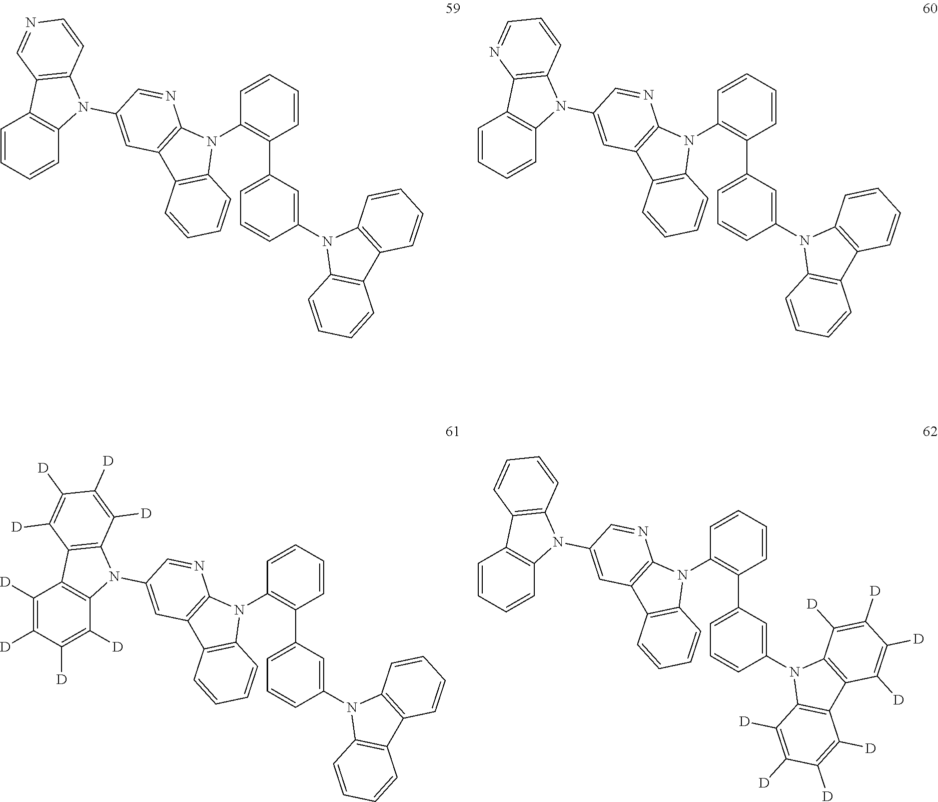 [SMILES: c1cc(-c2ccccc2-n2c3ccccc3c3cc(-n4c5ccccc5c5cnccc54)cnc32)cc(-n2c3ccccc3c3ccccc32)c1 ; c1cc(-c2ccccc2-n2c3ccccc3c3cc(-n4c5ccccc5c5ncccc54)cnc32)cc(-n2c3ccccc3c3ccccc32)c1 ; [2H]c1c([2H])c([2H])c2c(c1[2H])c1c([2H])c([2H])c([2H])c([2H])c1n2-c1cnc2c(c1)c1ccccc1n2-c1ccccc1-c1cccc(-n2c3ccccc3c3ccccc32)c1 ; [2H]c1c([2H])c([2H])c2c(c1[2H])c1c([2H])c([2H])c([2H])c([2H])c1n2-c1cccc(-c2ccccc2-n2c3ccccc3c3cc(-n4c5ccccc5c5ccccc54)cnc32)c1] 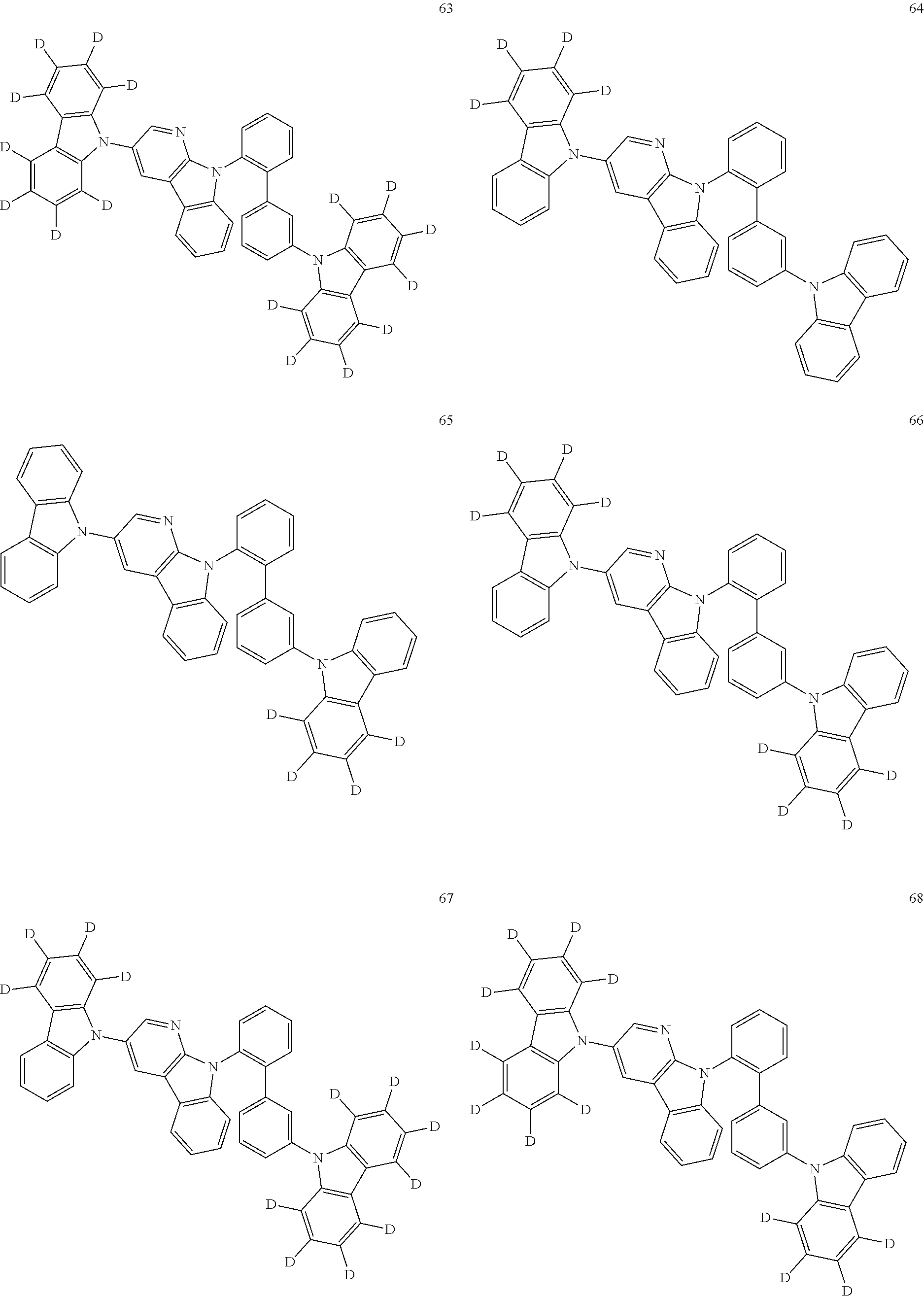 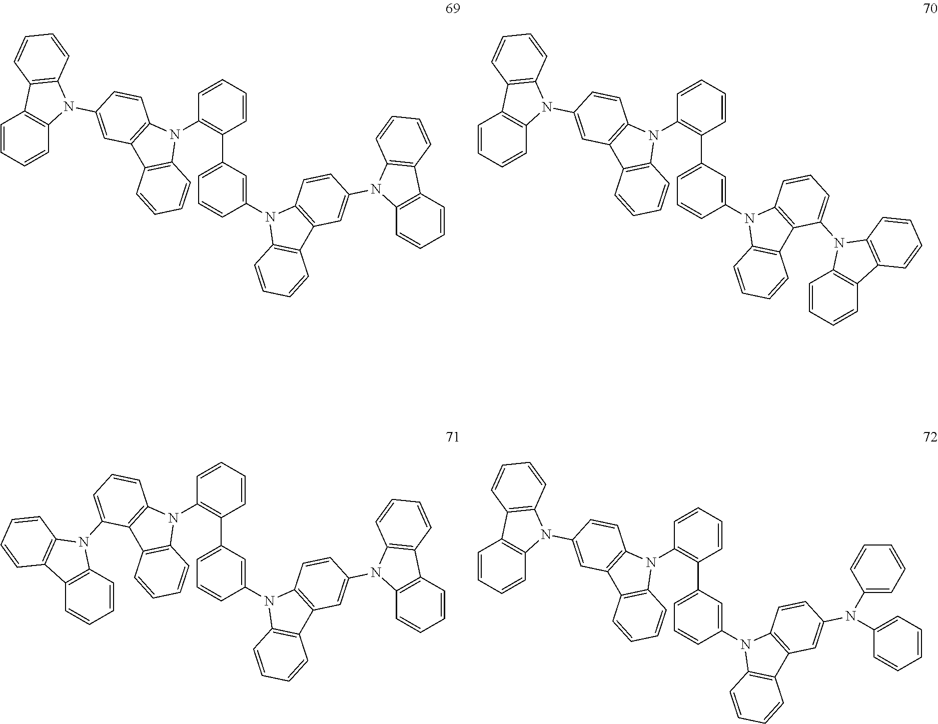 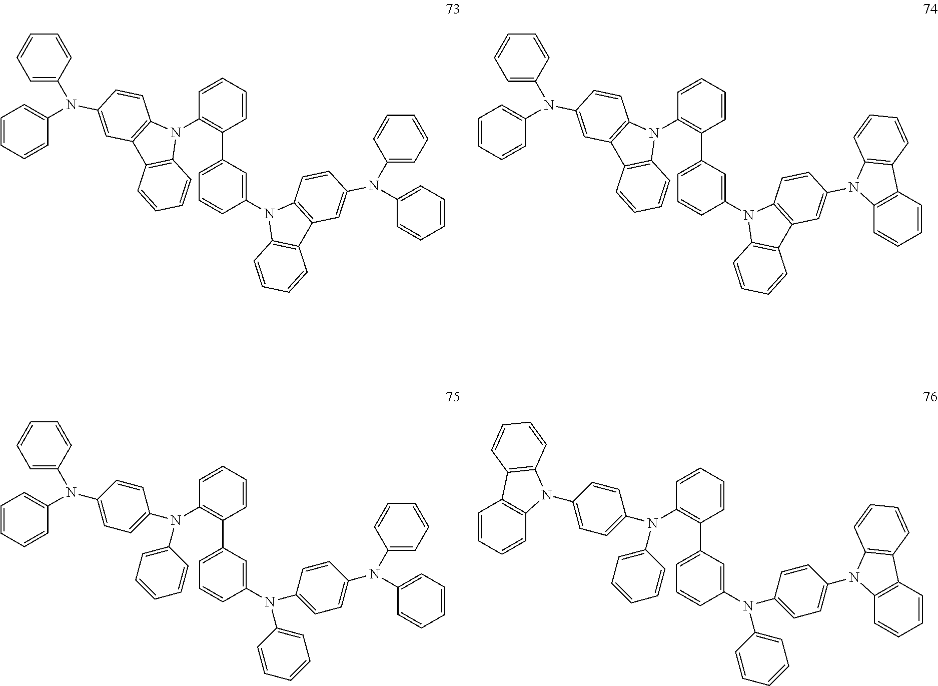 [SMILES: c1ccc(N(c2ccccc2)c2ccc3c(c2)c2ccccc2n3-c2cccc(-c3ccccc3-n3c4ccccc4c4cc(N(c5ccccc5)c5ccccc5)ccc43)c2)cc1 ; c1ccc(N(c2ccccc2)c2ccc3c(c2)c2ccccc2n3-c2ccccc2-c2cccc(-n3c4ccccc4c4cc(-n5c6ccccc6c6ccccc65)ccc43)c2)cc1 ; c1ccc(N(c2ccccc2)c2ccc(N(c3ccccc3)c3cccc(-c4ccccc4N(c4ccccc4)c4ccc(N(c5ccccc5)c5ccccc5)cc4)c3)cc2)cc1 ; c1ccc(N(c2ccc(-n3c4ccccc4c4ccccc43)cc2)c2cccc(-c3ccccc3N(c3ccccc3)c3ccc(-n4c5ccccc5c5ccccc54)cc3)c2)cc1] 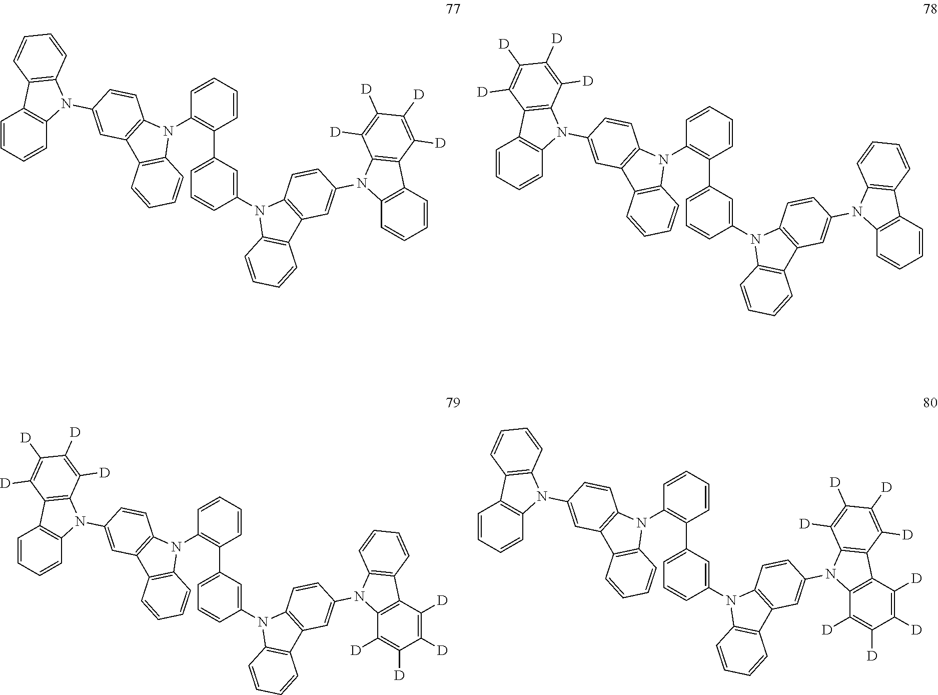 [chemical structure]
